(19) United States
(12) Patent Application Publication
(10) Pub. No.: US 2021/0316003 A1
(43) Pub. Date: Oct. 14, 2021

(54) BIOMARKERS FOR SACITUZUMAB GOVITECAN THERAPY

(71) Applicants: Thomas M. Cardillo, Cedar Knolls, NJ (US); Olivier Elemento, New York, NY (US); Bishoy M. Faltas, New York, NY (US); Trishna Goswami, Mendham, NJ (US); Thorsten Rj Sperber, Münster (DE); Scott T. Tagawa, New York, NY (US); Panagiotis J. Vlachostergios, New York, NY (US)

(72) Inventors: Thomas M. Cardillo, Cedar Knolls, NJ (US); Olivier Elemento, New York, NY (US); Bishoy M. Faltas, New York, NY (US); Trishna Goswami, Mendham, NJ (US); Thorsten Rj Sperber, Münster (DE); Scott T. Tagawa, New York, NY (US); Panagiotis J. Vlachostergios, New York, NY (US)

(73) Assignee: Immunomedics, Inc., Morris Plains, NJ (US)

(21) Appl. No.: 17/206,388

(22) Filed: Mar. 19, 2021

Related U.S. Application Data
(60) Provisional application No. 62/992,728, filed on Mar. 20, 2020.

(51) Int. Cl. A61K 47/68; C07K 16/30; A61K 45/06; A61K 31/4412; C12Q 1/6886

(52) U.S. Cl. CPC A61K 47/6803; C07K 16/30; A61K 45/06; C12Q 2600/118; C12Q 1/6886; C12Q 2600/106; A61K 31/4412

(57) ABSTRACT

The present invention relates to biomarkers of use for treating Trop-2 expressing cancer with an anti-Trop-2 ADC comprising an anti-Trop-2 antibody conjugated to an inhibitor of topoisomerase I, preferably SN-38 or DxD. The anti-Trop-2 ADC may be administered as a monotherapy or as a combination therapy with one or more anti-cancer agents, such as DDR inhibitors. Therapy with the ADC alone or in combination with other anti-cancer agents can reduce solid tumors in size, reduce or eliminate metastases and is effective to treat cancers resistant to standard therapies. Preferably, the combination therapy has an additive effect on inhibiting tumor growth. Most preferably, the combination therapy has a synergistic effect on inhibiting tumor growth. In specific embodiments, the biomarker may relate to a gene selected from the group consisting of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NDRG1, WEE1, PPP1R15A, MYBBP1A, SIRT1, ABL1, HRAS, ZNF385B, POLR2K and DDB2.

### CROSS-REFERENCE TO RELATED APPLICATIONS

This application claims the benefit under 35 U.S.C. § 119(e) of U.S. provisional application No. 62/992,728, filed on Mar. 20, 2020, which is hereby incorporated herein by reference in its entirety for all purposes.

### SEQUENCE LISTING

The instant application contains a Sequence Listing which has been submitted in ASCII format via EFS-Web and is hereby incorporated by reference in its entirety. Said ASCII copy, created on Mar. 17, 2021, is named IMM376-US-NP-SL.txt and is 1,667 bytes in size.

This patent application contains a lengthy table section. Copies of the tables have been submitted electronically in ASCII format and are hereby incorporated herein by reference, and may be employed in the practice of the methods provided herein. Said ASCII tables, created Dec. 3, 2019 are as follows: (1) IMM376-US-NP Appendix 1.txt, 10,198 bytes, (2) IMM376-US-NP Appendix 2—Part A.txt, 3,491 bytes, (3) IMM376-US-NP Appendix 2—Part B.txt, 96,588 bytes and (4) IMM376-US-NP Appendix 2—Part C.txt, 1,169 bytes.

LENGTHY TABLES
The patent application contains a lengthy table section. A copy of the table is available in electronic form from the USPTO web site (https://seqdata.uspto.gov/?pageRequest=docDetail&DocID=US20210316003A1). An electronic copy of the table will also be available from the USPTO upon request and payment of the fee set forth in 37 CFR 1.19(b)(3).

### FIELD

The present disclosure relates to use of anti-Trop-2 antibody-drug conjugates (ADCs), such as sacituzumab govitecan (IMMU-132), for treatment of Trop-2 expressing cancers. In certain embodiments, the ADC may be used with one or more diagnostic assays, for example a genomic assay to detect mutations or genetic variations, or a functional assay, such as Trop-2 expression levels, to predict sensitivity of the cancer to anti-Trop-2 ADC, alone or in combination with one or more other therapeutic agents, such as DDR (DNA damage response) inhibitors. In specific embodiments, a single genetic or physiological marker (collectively, “biomarker”), or a combination of two or more such biomarkers, may be of use to predict sensitivity of the cancer to particular combinations of ADC and other therapeutic agents. In preferred embodiments, the anti-Trop-2 antibody may be an hRS7 antibody, as described below. More preferably, the anti-Trop-2 antibody may be attached to a chemotherapeutic agent using a cleavable linker, such as a CL2A linker. Most preferably the drug is SN-38, and the ADC is sacituzumab govitecan (aka IMMU-132 or hRS7-CL2A-SN-38). However, other known anti-Trop-2 ADCs may be utilized, such as DS-1062. The disclosure is not limited as to the scope of combinations of agents of use for cancer therapy but may also include treatment with an ADC combined with any other known cancer treatment, including but not limited to PARP inhibitors, ATM inhibitors, ATR inhibitors, CHK1 inhibitors, CHK2 inhibitors, Rad51 inhibitors, WEE1 inhibitors, CDK 4/6 inhibitors, and/or platinum-based chemotherapeutic agents. In certain embodiments, the combination therapy may include an anti-Trop-2 ADC and one or more of the anti-cancer agents recited above. Preferably, the combination therapy, with or without biomarker analysis, is effective to treat resistant/relapsed cancers that are not susceptible to standard anti-cancer therapies, or that exhibit resistance to ADC monotherapy. The person of ordinary skill will be aware that the subject biomarkers are of use for a variety of purposes, such as increasing diagnostic accuracy, individualizing patient therapy (precision medicine), establishing a prognosis, predicting treatment outcomes and relapse, monitoring disease progression and/or identifying early relapse from cancer therapy. In specific embodiments, the biomarker may be selected from genetic markers in a DDR or an apoptosis gene, such as BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NDRG1, WEE1, PPP1R15A, MYBBP1A, SIRT1, ABL1, HRAS, ZNF385B, POLR2K or DDB2. Most preferably, one or more of the biomarkers may used to differentiate between responders and non-responders to an anti-Trop-2 ADC, such as sacituzumab govitecan or D-1062.

### BACKGROUND

Sacituzumab govitecan is an anti-Trop-2 antibody-drug conjugate (ADC) that has demonstrated efficacy against a wide range of Trop-2 expressing epithelial cancers, including but not limited to breast cancer, triple negative breast cancer (TNBC), HR+/HER2− metastatic breast cancer, urothelial cancer, small cell lung cancer (SCLC), non-small cell lung cancer (NSCLC), colorectal cancer, stomach cancer, bladder cancer, renal cancer, ovarian cancer, uterine cancer, endometrial cancer, prostate cancer, esophageal cancer and head-and-neck cancer (Ocean et al., 2017, Cancer 123:3843-54; Starodub et al., 2015, Clin Cancer Res 21:3870-78; Bardia et al., 2018, J Clin Oncol 36(15_suppl):1004).

Unlike most other current ADCs, sacituzumab govitecan (SG) is not conjugated to an ultratoxic drug or toxin (Cardillo et al., 2015, Bioconj Chem 26:919-31). Rather, SG comprises an anti-Trop-2 hRS7 antibody (e.g., U.S. Pat. Nos. 7,238,785; 8,574,575) conjugated via a CL2A linker (U.S. Pat. No. 7,999,083) to the active metabolite (SN38) of the topoisomerase I inhibitor, irinotecan. Perhaps due to the use of a lower toxicity conjugated drug, as well as the targeting effects of the anti-Trop-2 antibody, sacituzumab govitecan exhibits only moderate systemic toxicity, primarily neutropenia (Bardia et al., 2019, N Engl J Med 380:741-51) and has a highly favorable therapeutic window (Ocean et al., 2017, Cancer 123:3843-54; Cardillo et al., 2011, Clin Cancer Res 17:3157-69).

Sacituzumab govitecan is efficacious in second line or later treatment of diverse tumors, with activity in patients who are relapsed/refractory to standard chemotherapeutic agents and/or checkpoint inhibitors (Bardia et al., 2019, N Engl J Med 380:741-51; Faltas et al., 2016, Clin Genitourin Cancer 14:e75-9). For example, in a second line or later setting, phase I/II clinical trials with SG have reported a 33.3% response rate in metastatic TNBC, with a clinical benefit ratio of 45.5%, 5.5 months median progression-free survival (PFS) and overall survival (OS) of 13.0 months (Bardia et al., 2019, N Engl J Med 380:741-51). The patients treated with SG had previously failed therapy with taxanes, anthracyclines and other standard therapies, such as checkpoint inhibitor antibodies (Bardia et al., 2019, N Engl J Med 380:741-51).

Interim results have been published from a phase II open-label study of sacituzumab govitecan in patients with metastatic urothelial cancer (mUC) (Tagawa et al., 2019, Ann Oncol 30(suppl_5):v851-934, mdz394). Of 35 mUC patients treated with 10 mg/kg sacituzumab govitecan (SG), the objective response rate (ORR) was 29%, with 2 complete responses (CR), 6 confirmed partial responses (PR) and 2 PR pending confirmation. Seventy-four percent of treated patients demonstrated a reduction in tumor size. The ORR was 25% in patients with liver metastases. SG was well tolerated, with a manageable, predictable and consistent safety profile and no greater than or equal to grade 3 neuropathy, no interstitial lung disease and no treatment related deaths. These data built on earlier data generated in the first first-in-human study of sacituzunmab govitecan (IMMU-132-01) in which a ORR of 31% was reported in 45 urotherlial cancer patients treated at the recommended phase 2 dose of sacituzumab govitecan.

Clinical results with SG have also been obtained in patients with non-small cell lung cancer (NSCLC) (Heist et al., 2017, J Clin Oncol 35:2790-97). In 47 response assessable patients, treated with a median of three prior therapies (including checkpoint inhibitors), the ORR was 19%, with a clinical benefit rate of 43%. Median PFS was 5.2 months, with median OS of 9.5 months. A similar result was obtained in metastatic SCLC (Gray et al., 2017, Clin Cancer Res 23:5711-19). Of 53 mSCLC patients given SC, the ORR was 14%, with median response duration of 5.7 months, median PFS of 3.7 months and median OS of 7.5 months. Sixty percent of patients showed tumor shrinkage from baseline. Based on the results discussed above, it was concluded that SG is safe and efficacious for use in treating a wide variety of Trop-2+ cancers.

Despite these favorable responses to therapy with an anti-Trop-2 ADC, a substantial percentage of patients will still fail to respond or will develop resistance to monotherapy with the ADC. A need exists for a diagnostic assay, or a combination of assays, that can identify patients with tumors that may be more susceptible to treatment with anti-Trop-2 ADCs, such as sacituzumab govitecan, or to combination therapy with an ADC and one or more other known anti-cancer treatments. A further need exists for biomarkers that can identify patients with residual disease and/or at high risk of relapse who might benefit from therapy with the subject ADCs, alone or in combination with other agents.

### SUMMARY

In one aspect provided herein are methods for treating Trop-2 expressing cancers in a patient with anti-Trop-2 ADCs, either alone or in combination with at least one other known anti-cancer treatment. In some embodiments the methods provided herein involve the use of one or more biomarkers and assays before administering an anti-Trop2 ADC to a patient with Trop-2 expressing cancer. In some embodiments the methods involve the use of one or more biomarkers for the selection of patients for treatment with an anti-Trop2 ADC. In certain embodiments the methods provided herein involve use of one or more diagnostic assays to predict responsiveness of and/or to indicate a need for treatment of Trop-2 expressing cancers with anti-Trop-2 ADCs, either alone or in combination with at least one other known anti-cancer treatment. Such assays may detect the presence and/or absence of DNA or RNA biomarkers, such as mutations, promoter methylation, chromosomal rearrangements, gene amplification, and/or RNA splice variants. Alternatively, such assays may detect overexpression of mRNA and/or protein products of key genes, such as Trop-2. Genes of interest as biomarkers or for diagnostic assays may include, but are not limited to 53BP1, AKT1, AKT2, AKT3, APE1, ATM, ATR, BARD1, BAP1, BLM, BRAF, BRCA1, BRCA2, BRIP1 (FANCJ), CCND1, CCNE1, CCDKN1, CDK12, CHEK1, CHEK2, CK-19, CSA, CSB, DCLRE1C, DNA2, DSS1, EEPD1, EFHD1, EpCAM, ERCC1, ESR1, EXO1, FAAP24, FANC1, FANCA, FANCC, FANCD1, FANCD2, FANCE, FANCF, FANCM, HER2, HMBS, HR23B, KRT19, KU70, KU80, hMAM, MAGEA1, MAGEA3, MAPK, MGP, MLH1, MRE11, MRN, MSH2, MSH3, MSH6, MUC1-6, NBM, NBS1, NEK NF-κB, P53, PALB2, PARP1, PARP2, PIK3CA, PMS2, PTEN, RAD23B, RAD50, RAD51, RAD51 AP1, RAD51C, RAD51D, RAD52, RAD54, RAF, K-ras, H-ras, N-ms, RBBP8, c-myc, RIF1, RPA1, SCGB2A2, SLFN11, SLX1, SLX4, TMPRSS4, TP53, TROP-2, USP11, VEGF, WEE1, WRN, XAB2, XLF, XPA, XPC, XPD, XPF, XPG, XRCC4 and XRCC7. (See, e.g., Kwan et al., 2018, Cancer Discov 8:1286-99; Vardakis et al., 2010, Clin Cancer Res, 17:165-73; Lianidou & Markou, 2011, Clin Chem 57:1242-55; Xing et al., 2019, Breast Cancer Res 21:78; Banno et al., 2017, Int J Oncol 50:2049-58; Yaganeh et al., 2017, Genes Cancer 8:784-98; Kitazano et al., Cancer Sci, Jul. 30, 2019 (Epub ahead of print); Allegra et al., 2016, J Clin Oncol 34:179-85; Shaw et al., 2017, Clin Cancer Res 23:88-96; Jin et al., 2017, Cancer Biol Ther 18:369-78; Williamson et al., 2016, Nature Commun 7:13837; McCabe et al., 2006, Cancer Res 66:8109-15; Srivastava & Raghavan, 2015, Chem Biol 22:17-29). In more particular embodiments, genes of interest may be selected from BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NDRG1, WEE1, PPP1R15A, MYBBP1A, SIRT1, ABL1, HRAS, ZNF385B, POLR2K and DDB2.

Different forms of biomolecules may be detected, purified, and/or analyzed. In certain embodiments, cancer biomarkers may be detected by direct sampling (biopsy) of a suspected tumor, for example using immunohistochemistry, Western blotting, RT-PCR or other known techniques. Preferably, biomarkers may be detected in blood, lymph, serum, plasma, urine or other fluids (liquid biopsy). Biomarkers in liquid biopsy samples come in a variety of forms, such as proteins, cfDNA (cell-free DNA), ctDNA (circulating tumor DNA), and CTCs (circulating tumor cells) and each may be detected using specific advanced detection technologies discussed in detail below. While the methods and compositions disclosed herein are of use for detection, identification, characterization and/or prognosis of cancers in general, in more specific embodiments they may be applied to tumors that express a particular tumor-associated antigen (TAA), such as Trop-2. In such embodiments, the expression level or copy number of the TAA (e.g., Trop-2) may have predictive value independently of or in combination with other cancer biomarkers. Such predictive biomarkers may be of use to predict sensitivity or resistance to or toxicity of or need for treatment with ADC monotherapy or ADC combination therapy with other anti-cancer agents. Such biomarkers may also be of use to confirm the presence or absence of specific tumor types or to predict the course of disease in patients exhibiting specific biomarkers or combinations of biomarkers. Other uses of biomarkers include increasing diagnostic accuracy, individualizing patient therapy (precision medicine), monitoring disease progression and/or detecting early relapse from cancer therapy.

In certain embodiments, circulating tumor cells (CTCs) may be separated from blood, serum or plasma. The presence of CTCs in a patient's blood, plasma or serum may be predictive of metastatic cancer or indicative of residual cancer cells following earlier anti-cancer treatment. In addition to the diagnostic value of the presence of CTCs per se, the separated CTCs may also be assayed for the presence or absence of one or more biomarkers (see, e.g., Shaw et al., 2017, Clin Cancer Res 23:88-96; Tellez-Gabriel et al., 2019, Theranostics 9:4580-94; Kwan et al., 2018, Cancer Discov 8:1286-99). Techniques for separating CTCs from serum or plasma are discussed in more detail below, for example using a CELLSEARCH® system. Anti-Trop-2, anti-EpCAM or other known antibodies may be used as capture antibodies to isolate Trop-2+ or EpCAM+ CTCs. Alternatively, combinations of capture antibodies of use in CTC detection or separation are known and may be used.

In preferred embodiments, the invention involves combination therapy using an anti-Trop-2 ADC, in combination with one or more known anti-cancer agents. Such agents may include, but are not limited to, PARP inhibitors, ATM inhibitors, ATR inhibitors, CHK1 inhibitors, CHK2 inhibitors, Rad51 inhibitors, WEE1 inhibitors, PI3K inhibitors, AKT inhibitors, CDK 4/6 inhibitors, and/or platinum-based chemotherapeutic agents. Specific agents of use in combination therapy are discussed in more detail below, but may include olaparib, rucaparib, talazoparib, veliparib, niraparib, acalabrutinib, temozolomide, atezolizumab, pembrolizumab, nivolumab, ipilimumab, pidilizumab, durvalumab, BMS-936559, BMN-673, tremelimumab, idelalisib, imatinib, ibrutinib, eribulin mesylate, abemaciclib, palbociclib, ribociclib, trilaciclib, berzosertib, ipatasertib, uprosertib, afuresertib, triciribine, ceralasertib, dinaciclib, flavopiridol, roscovitine, G1T38, SHR6390, copanlisib, temsirolimus, everolimus, KU 60019, KU 55933, KU 59403, AZ20, AZD0156, AZD1390, AZD1775, AZD2281, AZD5363, AZD6738, AZD7762, AZD8055, AZD9150, BAY-937, BAY1895344, BEZ235, CCT241533, CCT244747, CGK 733, CID44640177, CID1434724, CID46245505, CHIR-124, EPT46464, FTC, VE-821, VRX0466617, VX-970, LY294002, LY2603618, M1216, M3814, M4344, M6620, MK-2206, NSC19630, NSC109555, NSC130813, NSC205171, NU6027, NU7026, prexasertib (LY2606368), PD0166285, PD407824, PV1019, SCH900776, SRA737, BMN 673, CYT-0851, mirin, Torin-2, fluoroquinoline 2, fumitremorgin C, curcurmin, Ko143, GF120918, YHO-13351, YHO-13177, XL9844, Wortmannin, lapatinib, sorafenib, sunitinib, nilotinib, gemcitabine, bortezomib, trichostatin A, paclitaxel, cytarabine, cisplatin, oxaliplatin and/or carboplatin.

More preferably, the combination therapy is more effective than the ADC alone, the anti-cancer agent alone, or the sum of the effects of ADC and anti-cancer agent. Most preferably, the combination exhibits synergistic effects for treatment of diseases, such as cancer, in human subjects. In alternative embodiments, the ADC or combination therapy may be used as a neoadjuvant or adjuvant therapy along with surgery, radiation therapy, chemotherapy, immunotherapy, radioimmunotherapy, immunomodulators, vaccines, and other standard cancer treatments.

In embodiments utilizing an anti-Trop-2 ADC, the anti-Trop-2 antibody moiety is preferably an hRS7 antibody, comprising the light chain CDR sequences CDR1 (KASQDVSIAVA, SEQ ID NOT); CDR2 (SASYRYT, SEQ ID NO:2); and CDR3 (QQHYITPLT, SEQ ID NO:3) and the heavy chain CDR sequences CDR1 (NYGMN, SEQ ID NO:4); CDR2 (WINTYTGEPTYTDDFKG, SEQ ID NO:5) and CDR3 (GGFGSSYWYFDV, SEQ ID NO:6). In more preferred embodiments, the anti-Trop-2 ADC is sacituzumab govitecan (hRS7-CL2A-SN-38). However, in alternative embodiments other known anti-Trop-2 ADCs may be utilized, as discussed below.

In a preferred embodiment, a drug moiety conjugated to a subject antibody to form an ADC is the active metabolite of a topoisomerase I inhibitor, SN-38 (Moon et al., 2008, J Med Chem 51:6916-26) or DxD (Ogitani et al., 2016 Clin Cancer Res 22:5097-108; Ogitani et al., 2016 Bioorg Med Chem Lett 26:5069-72). However, other drug moieties that may be utilized include taxanes (e.g., baccatin III, taxol), auristatins (e.g., MMAE), calicheamicins, epothilones, anthracyclines (e.g., doxorubicin (DOX), epirubicin, morpholinodoxorubicin, cyanomorpholino-doxorubicin, 2-pyrrolinodoxorubicin), topotecan, etoposide, cisplatin, oxaliplatin, or carboplatin (see, e.g., Priebe W (ed.), 1995, ACS symposium series 574, published by American Chemical Society, Washington D.C., (332 pp); Nagy et al., 1996, Proc. Natl. Acad. Sci. USA 93:2464-2469). Generally, any anti-cancer cytotoxic drug, more preferably a drug that results in DNA damage may be utilized. Preferably, the antibody or fragment thereof links to at least one chemotherapeutic drug moiety; preferably 1 to 5 drug moieties; more preferably 6 to 12 drug moieties, most preferably about 6 to about 8 drug moieties.

Various embodiments may concern use of the subject methods and compositions to treat a cancer, including but not limited to oral, esophageal, gastrointestinal, lung, stomach, colon, rectal, breast, ovarian, prostatic, pancreatic, uterine, endometrial, cervical, urinary bladder, bone, brain, connective tissue, thyroid, liver, gall bladder, urothelial, renal, skin, central nervous system and testicular cancer. Preferably, the cancer may be metastatic triple-negative breast cancer (TNBC), metastatic HR+/HER2− breast cancer, metastatic non-small-cell lung cancer, metastatic small-cell lung cancer, metastatic endometrial cancer, metastatic urothelial cancer, metastatic pancreatic cancer, metastatic prostate cancer or metastatic colorectal cancer. The cancer to be treated may be metastatic or non-metastatic and the subject therapy may be used in a first-line, second-line, third-line or later stage cancer and in a neoadjuvant, adjuvant metastatic or maintenance setting. In some embodiments the cancer is urothelial cancer. In some embodiments the cancer is metatstatic urothelial cancer. In some embodiments the cancer is treatment resistant urothelial cancer. In some embodiments the cancer is resistant to treatment with platinum-based and/or checkpoint inhibitor (CPI) (e.g., anti-PD1 antibody or anti-PD-L1 antibody) based therapy. In some embodiments the cancer is metastatic TNBC.

Preferred optimal dosing of ADCs may include a dosage of between 4 to 16 mg/kg, preferably 6 to 12 mg/kg, more preferably 8 to 10 mg/kg, given either weekly, twice weekly, every other week, or every third week. The optimal dosing schedule may include treatment cycles of two consecutive weeks of therapy followed by one, two, three or four weeks of rest, or alternating weeks of therapy and rest, or one week of therapy followed by two, three or four weeks of rest, or three weeks of therapy followed by one, two, three or four weeks of rest, or four weeks of therapy followed by one, two, three or four weeks of rest, or five weeks of therapy followed by one, two, three, four or five weeks of rest, or administration once every two weeks, once every three weeks or once a month. Treatment may be extended for any number of cycles. Exemplary dosages of use may include 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, or 18 mg/kg. The person of ordinary skill will realize that a variety of factors, such as age, general health, specific organ function or weight, as well as effects of prior therapy on specific organ systems (e.g., bone marrow) and the intent of therapy (curative or palliative) may be considered in selecting an optimal dosage and schedule of ADC, and that the dosage and/or frequency of administration may be increased or decreased during the course of therapy. The dosage may be repeated as needed, with evidence of tumor shrinkage observed after as few as 4 to 8 doses. The use of combination therapies can allow lower doses of each therapeutic to be given in such combinations, thus reducing certain severe side effects, and potentially reducing the courses of therapy required. When there is no or minimal overlapping toxicity, full doses of each can also be given.

The claimed methods provide for shrinkage of solid tumors, of 15% or more, preferably 20% or more, preferably 30% or more, more preferably 40% or more in size (as measured by summing the longest diameter of target lesions, as per RECIST or RECIST 1.1). The person of ordinary skill will realize that tumor size may be measured by a variety of different techniques, such as total tumor volume, maximal tumor size in any dimension or a combination of size measurements in several dimensions. This may be with standard radiological procedures, such as computed tomography, magnetic resonance imaging, ultrasonography, and/or positron-emission tomography. The means of measuring size is less important than observing a trend of decreasing tumor size with antibody or immunoconjugate treatment, preferably resulting in elimination of the tumor. However, to comply with RECIST guidelines, CT or MRI with contrast is preferred on a serial basis, and should be repeated to confirm measurements. For hematological malignancies, imaging as above as well as other standard measure for cancer response may be utilized, such as cell counts of different cell populations, detection and/or level of circulating tumor cells, immunohistology, cytology or fluorescent microscopy and similar techniques.

The optimized dosages and schedules of administration disclosed herein, used with or without biomarker analysis, show unexpected superior efficacy and reduced toxicity in human subjects, which could not have been predicted from animal model studies. Surprisingly, the superior efficacy allows treatment of tumors that were previously found to be resistant to one or more standard anti-cancer therapies, including some tumors that failed prior treatment with the irinotecan parent compound of SN-38.

### BRIEF DESCRIPTION OF THE FIGURES

FIG. 1A. Treatment response among patients with metastatic urothelial cancer treated with sacituzumab govitecan. Waterfall plot showing best percent change from baseline in the sum of the diameters of the target lesions* in 40 patients (excludes 5 patients with no post-baseline assessments). Abbreviations: CR, complete response; PR, partial response; SD, stable disease; PD, progressive disease. *Sum of the diameters of the target lesions (longest for non-nodal, short axis for nodal lesions); ^{†}0% change with best overall response of PD; ^{‡}Target lesions shrinkage >30% but unconfirmed, hence classified as SD; ^{§} CR based on lymph node target lesions shrinkage to <10 mm; **100% reduction of target lesions, but stable persistence of a non-target lesion, hence classified as PR.

FIG. 1B. Treatment response among patients with metastatic urothelial cancer treated with sacituzumab govitecan. Swimmer plot of patients achieving objective response (n=14) from start of treatment to progression. Black boxes indicate onset of response, and arrows indicate ongoing response at data cut-off. Black circles indicate patients whose duration of responses were censored due to missing 2 tumor assessments or to discontinuation. At the time of analysis, 3 patients were still on treatment with an ongoing response (>17 months, >19 months, and >29 months).

FIG. 2A. Median progression-free (PFS) among patients with metastatic urothelial cancer treated with sacituzumab govitecan.

FIG. 2B. Median overall survival (OS) among patients with metastatic urothelial cancer (mUC) treated with sacituzumab govitecan.

FIG. 3A. Molecular features associated with response to sacituzumab govitecan. Oncoprint demonstrating the frequency of mutations in DNA Damage Repair (DDR) and apoptosis genes in the G0:0097193 signaling pathway in 14 mUC patients treated with sacituzumab govitecan (responders n=6, non-responders n=8).

FIG. 3B. Molecular features associated with response to sacituzumab govitecan in mUC patients. RNAseq heatmap showing differentially expressed genes between responders versus non-responders (False Discovery Rate [FDR]<0.001; upregulated genes: log fold change [LFC]>2, n=374; down-regulated genes: LFC<−2, n=380).

FIG. 3C. Molecular features associated with response to sacituzumab govitecan in mUC patients. Differences in single-sample GSEA (ssGSEA) enrichment scores showing the enrichment of apoptosis and P53 pathways in responders versus non-responders. Mann-Whitney test p-values are reported.

FIG. 4A. Response and treatment analyses in TNBC. Waterfall plot showing best percent change from baseline in the sum of target lesion diameters (longest diameter for non-nodal lesions and short axis for nodal lesions). Asterisks denote 3 patients whose best percent change is zero percent (2 SD, 1 PD). The dashed lines at 20% and −30% indicate progressive disease and partial response, respectively, according to RECIST.

FIG. 4B. Swimmer plot of the objective responses (according to RECIST, version 1.1) in TNBC patients from start of treatment to disease progression, as determined by local assessment. At the time of the analysis, 6 patients had a continuing response. The vertical dashed lines show the response at 6 months and 12 months.

FIG. 5A. Graphic representation of anti-tumor response and duration in response-assessable mSCLC patients. Best percentage change in the sum of the diameters for the selected target lesion and best overall response descriptor according to RECIST 1.1 criteria. Patients are identified with respect to the sacituzumab govitecan starting dose and whether they were sensitive or resistant to prior first-line therapy. Patient with unconfirmed partial responses failed to maintain at least a 30% tumor reduction on their next CT assessment 4-6 weeks after the first observed objective response. The best overall response for these patients by RECIST 1.0 is stable disease.

FIG. 5B. Graphic representation of anti-tumor response and duration in response-assessable mSCLC patients. Duration of response from the start of treatment for those patients who achieved partial or complete response. Timing when tumor shrinkage achieved ≥30% is shown, along with sacituzumab govitecan starting dose and sensitivity to first-line therapy.

FIG. 5C. Graphic representation of anti-tumor response and duration in mSCLC response-assessable patients. Dynamics of response for patients who achieved stable disease or better. Two patients with confirmed partial responses who are continuing treatment are shown with dashed line

FIG. 6A. Kaplan-Meier derived progression-free survival curves for all 53 mSCLC patients enrolled in the sacituzumab govitecan trial.

FIG. 6B. Kaplan-Meier derived overall survival curves for all 53 mSCLC patients enrolled in the sacituzumab govitecan trial.

### DETAILED DESCRIPTION

### Definitions

In the description that follows, a number of terms are used and the following definitions are provided to facilitate understanding of the claimed subject matter. Terms that are not expressly defined herein are used in accordance with their plain and ordinary meanings.

Unless otherwise specified, a or an means “one or more.”

The term about is used herein to mean plus or minus ten percent (10%) of a value. For example, “about 100” refers to any number between 90 and 110.

An antibody, as used herein, refers to a full-length (i.e., naturally occurring or formed by normal immunoglobulin gene fragment recombinatorial processes) immunoglobulin molecule (e.g., an IgG antibody). An antibody may be conjugated or otherwise derivatized within the scope of the claimed subject matter. Such antibodies include but are not limited to IgG1, IgG2, IgG3, IgG4 (and IgG4 subforms), as well as IgA isotypes. As used below, the abbreviation “MAb” may be used interchangeably to refer to an antibody, antibody fragment, monoclonal antibody or multispecific antibody.

An antibody fragment is a portion of an antibody such as F(ab′)₂, F(ab)₂, Fab′, Fab, Fv, scFv (single chain Fv), single domain antibodies (DABs or VHHs) and the like, including half-molecules of IgG4 (van der Neut Kolfschoten et al. (Science, 2007; 317:1554-1557). Regardless of structure, an antibody fragment of use binds with the same antigen that is recognized by the intact antibody. The term “antibody fragment” also includes synthetic or genetically engineered proteins that act like an antibody by binding to a specific antigen to form a complex. For example, antibody fragments include isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). The fragments may be constructed in different ways to yield multivalent and/or multispecific binding forms.

A therapeutic agent is an atom, molecule, or compound that is useful in the treatment of a disease. Examples of therapeutic agents include, but are not limited to, antibodies, antibody fragments, immunoconjugates, checkpoint inhibitors, drugs, cytotoxic agents, pro-apoptotic agents, toxins, nucleases (including DNAse and RNAse), hormones, immunomodulators, chelators, ‘photoactive agents or dyes, radionuclides, oligonucleotides, interference RNA, siRNA, RNAi, anti-angiogenic agents, chemotherapeutic agents, cytokines, chemokines, prodrugs, enzymes, binding proteins or peptides or combinations thereof.

As used herein, where reference is made to increased or decreased expression of a particular gene, the term refers to an increase or decrease in a cancer cell compared to normal, benign and/or wild-type cells.

Antibodies and Antibody-Drug Conjugates (ADCs)

Certain embodiments relate to use of anti-cancer antibodies, either in unconjugated form or else as an immunoconjugate (e.g., an ADC) attached to one or more therapeutic agents. Preferably the conjugated agent is one that induces DNA strand breaks, more preferably by inhibiting topoisomerase I. Exemplary inhibitors of topoisomerase I include SN-38 and DxD. However, other topoisomerase I inhibitors are known in the art and any such known topoisomerase I inhibitors may be used in an anti-Trop-2 ADC. Exemplary topoisomerase I inhibitors include the camptothecins, such as irinotecan, topotecan, SN-38, diflomotecan, S39625, silatecan, belotecan, namitecan, gimatecan, belotecan or camptothecin, as well as non-camptothecins, such as indolocarbazole, phenanthridine, indenoisoquinoline, and their derivatives, such as NSC 314622, NSC 725776, NSC 724998, ARC-111, isoindolo[2,1-a]quinoxalines, indotecan, indimitecan, CRLX101, rebeccamycin, edotecarin, or becatecarin. [See, e.g., Hevener et al., 2018, Acta Pharm Sin B 8:844-61]

In alternative embodiments, a topoisomerase II inhibitor may be utilized, such as anthracyclines, doxorubucin, epirubicin, valrubicin, daunorubicin, idarubicin, aldoxorubicin, anthracenediones, mitoxantrone, pixantrone, amsacrine, dexrazoxane, epipodophyllotoxins, ciprofloxacin, vosaroxin, teniposide or etoposide. [See, e.g., Hevener et al., 2018, Acta Pharm Sin B 8:844-61]

Although topoisomerase inhibitors are preferred for antibody conjugation, other agents that induce DNA damage and/or strand breaks are known and may be utilized in alternative embodiments. Such known anti-cancer agents include, but are not limited to, nitrogen mustards, folate analogs such as aminopterin or methotrexate, alkylating agents such as cyclophosphamide, chlorambucil, mitomycin C, streptozotocin or melphalan, nitrosoureas such as carmustine, lomustine or semustine, triazenes such as dacarbazine or temozolomide, or platinum-based inhibitors such as cisplatin, carboplatin, picoplatin or oxaliplatin. [See, e.g., Ong et al., 2013, Chem Biol 20:648-59]

In a preferred embodiment, antibodies or immunoconjugates comprising an anti-Trop-2 antibody, such as the hRS7 antibody, can be used to treat carcinomas such as carcinomas of the esophagus, pancreas, lung, stomach, colon, rectum, urinary bladder, urothelium, breast, ovary, cervix, endometrium, uterus, kidney, head-and-neck, brain and prostate, as disclosed in U.S. Pat. Nos. 7,238,785; 7,999,083; 8,758,752; 9,028,833; 9,745,380; and 9,770,517; the Examples section of each incorporated herein by reference. An hRS7 antibody is a humanized antibody that comprises light chain complementarity-determining region (CDR) sequences CDR1 (KASQDVSIAVA, SEQ ID NO:1); CDR2 (SASYRYT, SEQ ID NO:2); and CDR3 (QQHYITPLT, SEQ ID NO:3) and heavy chain CDR sequences CDR1 (NYGMN, SEQ ID NO:4); CDR2 (WINTYTGEPTYTDDFKG, SEQ ID NO:5) and CDR3 (GGFGSSYWYFDV, SEQ ID NO:6). However, in alternative embodiments other anti-Trop-2 antibodies are known and may be utilized in an anti-Trop-2 ADC. Exemplary anti-Trop-2 antibodies include, but are not limited to, catumaxomab, VB4-845, IGN-101, adecatumumab, ING-1, EMD 273 066 or hTINA1 (see U.S. Pat. No. 9,850,312). Anti-Trop-2 antibodies are commercially available from a number of sources and include LS-C126418, LS-C178765, LS-C126416, LS-C126417 (LifeSpan BioSciences, Inc., Seattle, Wash.); 10428-MM01, 10428-MM02, 10428-R001, 10428-R030 (Sino Biological Inc., Beijing, China); MR54 (eBioscience, San Diego, Calif.); sc-376181, sc-376746, Santa Cruz Biotechnology (Santa Cruz, Calif.); MM0588-49D6, (Novus Biologicals, Littleton, Colo.); ab79976, and ab89928 (ABCAM®, Cambridge, Mass.).

Other anti-Trop-2 antibodies have been disclosed in the patent literature. For example, U.S. Publ. No. 2013/0089872 discloses anti-Trop-2 antibodies K5-70 (Accession No. FERM BP-11251), K5-107 (Accession No. FERM BP-11252), K5-116-2-1 (Accession No. FERM BP-11253), T6-16 (Accession No. FERM BP-11346), and T5-86 (Accession No. FERM BP-11254), deposited with the International Patent Organism Depositary, Tsukuba, Japan. U.S. Pat. No. 5,840,854 disclosed the anti-Trop-2 monoclonal antibody BR110 (ATCC No. HB11698). U.S. Pat. No. 7,420,040 disclosed an anti-Trop-2 antibody produced by hybridoma cell line AR47A6.4.2, deposited with the ID AC (International Depository Authority of Canada, Winnipeg, Canada) as accession number 141205-05. U.S. Pat. No. 7,420,041 disclosed an anti-Trop-2 antibody produced by hybridoma cell line AR52A301.5, deposited with the ID AC as accession number 141205-03. U.S. Publ. No. 2013/0122020 disclosed anti-Trop-2 antibodies 3E9, 6G11, 7E6, 15E2, 18B1. Hybridomas encoding a representative antibody were deposited with the American Type Culture Collection (ATCC), Accession Nos. PTA-12871 and PTA-12872. U.S. Pat. No. 8,715,662 discloses anti-Trop-2 antibodies produced by hybridomas deposited at the AID-ICLC (Genoa, Italy) with deposit numbers PD 08019, PD 08020 and PD 08021. U.S. Patent Application Publ. No. 20120237518 discloses anti-Trop-2 antibodies 77220, KM4097 and KM4590. U.S. Pat. No. 8,309,094 (Wyeth) discloses antibodies A1 and A3, identified by sequence listing. U.S. Pat. No. 9,850,312 disclosed the anti-Trop-2 antibodies TINA1, cTINA1 and hTINA1. The Examples section of each patent or patent application cited above in this paragraph is incorporated herein by reference. Non-patent publication Lipinski et al. (1981, Proc Natl. Acad Sci USA, 78:5147-50) disclosed anti-Trop-2 antibodies 162-25.3 and 162-46.2.

In a preferred embodiment, the antibodies that are used in the treatment of human disease are human or humanized (CDR-grafted) versions of antibodies, although murine and chimeric versions of antibodies can be used. Same species IgG molecules as delivery agents are mostly preferred to minimize immune responses. This is particularly important when considering repeat treatments. For humans, a human or humanized IgG antibody is less likely to generate an anti-IgG immune response from patients.

Formulation and Administration of ADCs

Antibodies or immunoconjugates (e.g., ADCs) can be formulated according to known methods to prepare pharmaceutically useful compositions, whereby the antibody or immunoconjugate is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

In a preferred embodiment, the antibody or immunoconjugate is formulated in Good's biological buffer (pH 6-7), using a buffer selected from the group consisting of N-(2-acetamido)-2-aminoethanesulfonic acid (ACES); N-(2-acetamido)iminodiacetic acid (ADA); N,N-bis(2-hydroxyethyl)-2-aminoethanesulfonic acid (BES); 4-(2-hydroxyethyl)piperazine-1-ethanesulfonic acid (HEPES); 2-(N-morpholino)ethanesulfonic acid (MES); 3-(N-morpholino)propanesulfonic acid (MOPS); 3-(N-morpholinyl)-2-hydroxypropanesulfonic acid (MOPSO); and piperazine-N,N′-bis(2-ethanesulfonic acid) [Pipes]. More preferred buffers are MES or MOPS, preferably in the concentration range of 20 to 100 mM, more preferably about 25 mM. Most preferred is 25 mM MES, pH 6.5. The formulation may further comprise 25 mM trehalose and 0.01% v/v polysorbate 80 as excipients, with the final buffer concentration modified to 22.25 mM as a result of added excipients. The preferred method of storage is as a lyophilized formulation of the conjugates, stored in the temperature range of −20° C. to 2° C., with the most preferred storage at 2° C. to 8° C.

The antibody or immunoconjugate can be formulated for intravenous administration via, for example, bolus injection, slow infusion or continuous infusion. Preferably, the antibody of the present invention is infused over a period of less than about 4 hours, and more preferably, over a period of less than about 3 hours. For example, the first 25-50 mg could be infused within 30 minutes, preferably even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Generally, the dosage of an administered antibody or immunoconjugate for humans will vary depending upon such factors as the patient's age, weight, height, sex, general medical condition and previous medical history. It may be desirable to provide the recipient with a dosage of immunoconjugate that is in the range of from about 1 mg/kg to 24 mg/kg as a single intravenous infusion, although a lower or higher dosage also may be administered as circumstances dictate. The dosage may be repeated as needed, for example, once per week for 4-10 weeks, once per week for 8 weeks, or once per week for 4 weeks. It may also be given less frequently, such as every other week for several months, or monthly or quarterly for many months, as needed in a maintenance therapy. Preferred dosages may include, but are not limited to, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, and 18 mg/kg. The dosage is preferably administered multiple times, once or twice a week, or as infrequently as once every 3 or 4 weeks. A minimum dosage schedule of 4 weeks, more preferably 8 weeks, more preferably 16 weeks or longer may be used. The schedule of administration may comprise administration once or twice a week, on a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five week off; (vi) four weeks of therapy followed by one, two, three, four or five week off; (vii) five weeks of therapy followed by one, two, three, four or five week off; (viii) monthly and (ix) every 3 weeks. The cycle may be repeated 2, 4, 6, 8, 10, 12, 16 or 20 times or more.

Alternatively, an antibody or immunoconjugate may be administered as one dosage every 2 or 3 weeks, repeated for a total of at least 3 dosages. Or, twice per week for 4-6 weeks. If the dosage is lowered to approximately 200-300 mg/m² (340 mg per dosage for a 1.7-m patient, or 4.9 mg/kg for a 70 kg patient), it may be administered once or even twice weekly for 4 to 10 weeks. Alternatively, the dosage schedule may be decreased, namely every 2 or 3 weeks for 2-3 months. It has been determined, however, that even higher doses, such as 12 mg/kg once weekly or once every 2-3 weeks can be administered by slow i.v. infusion, for repeated dosing cycles. The dosing schedule can optionally be repeated at other intervals and dosage may be given through various parenteral routes, with appropriate adjustment of the dose and schedule.

DNA Damage and Repair Pathways

Use of anti-cancer ADCs with drug moieties targeted against topoisomerases can result in accumulation of single- or double-stranded breaks in cancer cell DNA. Resistance to or relapse from the anti-cancer effects of topoisomerase I inhibitors, or other anti-cancer agents that damage DNA, may result from the existence of DNA repair mechanisms, such as the DNA damage response (DDR). DDR is a complex set of pathways responsible for repair of damage to DNA in normal and tumor cells. Inhibitors directed against DDR pathways may be utilized in combination with anti-Trop-2 ADCs to provide increased anti-cancer efficacy in tumors that are relapsed from or resistant to monotherapy with anti-Trop-2 ADCs. Alternatively, combination therapy may be used in a first-line therapy if the combination is substantially superior to monotherapy with ADC or other therapeutic agent alone. In addition, the presence of mutations, other genetic defects or changes in expression levels of genes encoding DDR components may be predictive of the efficacy of anti-Trop-2 ADCs and/or of combination therapy with an anti-Trop-2 ADC and one or more other anti-cancer agents.

In preferred embodiments, the subject ADCs may be used in combination with one or more known anti-cancer agents that inhibit various steps in the DDR pathways. There are numerous pathways involved in cellular DNA repair, with partial overlap in the protein effectors of the different pathways. Use of topoisomerase-inhibiting ADCs in combination with other inhibitors directed against different steps in the DNA damage repair pathways may exhibit synthetic lethality, wherein simultaneous loss of function in two different genes results in cell death, whereas loss of function in just one gene does not (e.g., Cardillo et al., 2017, Clin Cancer Res 23:3405-15). The concept may also be applied in cancer therapy, wherein a cancer cell carrying a mutation in one gene is targeted by a chemotherapeutic agent that inhibits the function of a second gene used by the cell to overcome the first mutation (Cardillo et al., 2017, Clin Cancer Res 23:3405-15). This concept has been applied, for example, to use of PARP inhibitors in cells bearing BRCA gene mutations (Benafif & Hall, 2015, Onco Targets Ther 8:519-28). In principle, synthetic lethality may be applied with or without the presence of underlying cancer cell mutations, for example by using combination therapy with two or more inhibitors targeted against different aspects of DDR pathways, alone or in combination with DNA damage-inducing agents.

Double-strand DNA breaks (DSBs) are repaired by two major pathways—homologous recombination (HR) and nonhomologous end joining (NHEJ). [See, e.g., Srivastava & Raghavan, 2015, Chem Biol 22:17-29] Each of these comprises subpathways—classical or alternative subpathways for NHEJ (respectively, cNHEJ and aNHEJ) and single-strand annealing (SSA) for the HR pathway. HR requires extensive homology for repair of DSBs and is most active in the S and G2 phases of the cell cycle, while NHEJ utilizes limited or no homology for end joining and can act throughout the cell cycle (Srivastava & Raghavan, 2015, Chem Biol 22:17-29).

Activation of DDR pathways by DSB includes checkpoint arrest, mediated via ATM, ATR and DNA-PKcs (Nickoloff et al., 2017, J Natl Cancer Inst 109:djx059). ATM is required for DSB repair by HR and triggers DSB end resection by stimulating nucleolytic activity of CtIP and MRE11 to generate 3′-ssDNA overhangs, followed by RPA loading and RAD51 nucleofilament formation (Bakr et al., 2015, Nucleic Acids Res 43:3154). ATR responds to a broader spectrum of DNA damage, including DSBs and ssDNA (Marechal et al., 2013, Cold Spring Harb Perspect Biol 5:a012716). However, the functions of ATR and ATM are not mutually exclusive, and both are required for DSB-induced checkpoint responses and DSB repair (Marechal et al., 2013, Cold Spring Harb Perspect Biol 5:a012716). Localization of the ATR-ATRIP complex to sites of DNA damage is dependent on the presence of long stretches of RPA-coated ssDNA, which may be generated by resection as discussed below (Marechal et al., 2013, Cold Spring Harb Perspect Biol 5:a012716). DNA-PKcs is the catalytic subunit of DNA-PK and is primarily involved in the NHEJ pathway (Marechal et al., 2013, Cold Spring Harb Perspect Biol 5:a012716).

Determination of which DSB repair pathway is utilized is mediated in part by the amount of 5′ end resection at the DSB, which is inhibited by 53BP1/RIF1 and promoted by BRCA1/CtIP. Increased resection favors the HR repair pathways, while decreased resection promotes the NHEJ pathways (Nickoloff et al., 2017, J Natl Cancer Inst 109:djx059). At the start of the HR pathways, MRE11 (part of the MRN complex along with RAD50 and NBS1) initiates limited end resection, which is followed by Exo1/EEPD1 and Dna2 for extensive resection (Nickoloff et al., 2017, J Natl Cancer Inst 109:djx059). In the NHEJ pathways, 53BP1/RIF1 and KU70/80 inhibit resection and promote classical NHEJ, while PARP1 competes with the KU proteins and promotes limited end resection for alternative NHEJ (Nickoloff et al., 2017, J Natl Cancer Inst 109:djx059). Pol 9 is also involved in aNHEJ.

Further steps in the HR pathway are promoted by RPA, BRCA2, RAD51, RAD52, RAD54, and Pol δ (Nickoloff et al., 2017, J Natl Cancer Inst 109:djx059). RAD52 is also involved in SSA, along with ERCC1, ERCC2, ERCC3 and ERCC4 (Nickoloff et al., 2017, J Natl Cancer Inst 109:djx059). Other proteins involved in HR include RAD50, NBS1, BLM, XPF, FANCM, FAAP24, FANC1, FAND2, MSH3, SLX4, MUS81, EME1, SLX1, PALB2, BRIP1, BARD1, BAP1, PTEN, RAD51C, USP11, WRN and NER. [Nickoloff et al., 2017, J Natl Cancer Inst 109:djx059, Srivastava & Raghavan, 2015, Chem Biol 22:17-29] Other proteins involved in NHEJ include Artemis, Pol μ, Pol λ, Ligase IV, XRCC4, and XLF. [Nickoloff et al., 2017, J Natl Cancer Inst 109:djx059, Srivastava & Raghavan, 2015, Chem Biol 22:17-29] Further details regarding the roles of these various DDR proteins and inhibitors for each are provided below.

Repair of single-stranded DNA lesions can also occur via multiple pathways—base excision repair (BER), nucleotide excision repair (NER) and mismatch repair (MMR). The BER pathway is facilitated by APE1, PARP1, Pol β, Lig III and XRCC1. NER is facilitated by XPC, RAD23B, HR23B, XPF, ERCC1, XPG, XPA, RPA, XPD, CSA, CSB, XAB2 and Pol δ/κ/ε. MMR is facilitated by MutSα/β, MLH1, PMS2, Exo1, PARP1, MSH2, MSH6 and Pol δ/ε (Nickoloff et al., 2017, J Natl Cancer Inst 109:djx059). Mutations in MSH2 predispose cancers to sensitivity to methotrexate and psoralen (Nickoloff et al., 2017, J Natl Cancer Inst 109:djx059). Defects in NER, such as decreased expression of ERCC1, predispose to sensitivity to cross-linking agents such as cisplatin as well as PARP1 or ATR inhibitors (Nickoloff et al., 2017, J Natl Cancer Inst 109:djx059).

As discussed below, inhibitors of various of these DDR proteins are known, and any such known inhibitor may be utilized in combination with a subject ADC. In more preferred embodiments, the presence of mutations in BRCA1 and/or BRCA2 may be predictive of efficacy of either ADC monotherapy or combination therapy with an ADC and an inhibitor of DSB repair.

Combination Therapy with ADCs and Inhibitors of DNA Damage Repair

As discussed above, a key objective of combination therapy with anti-Trop-2 ADCs, together with one or more inhibitors of DDR pathways, is to induce an artificial (as opposed to genetic) synthetic lethality, where the combination of agents that produce DNA damage (e.g., topoisomerase I inhibitors) with agents that inhibit steps in the DDR damage repair pathways is effective to kill cancer cells that are resistant to either type of agent alone. DDR inhibitors of particular interest for combination therapies are directed against PARP, ATR, ATM, CHK1, CHK2, CDK12, RAD51, RAD52 and WEE1. In alternative embodiments, the DDR inhibitor of interest may be a DDR inhibitor that is not a PARP inhibitor or RAD51 inhibitor.

PARP Inhibitors

Poly-(ADP-ribose) polymerase (PARP) plays a key role in the DNA damage response and either directly or indirectly affects numerous DDR pathways, including BER, HR, NER, NHEJ and MMR (Gavande et al., 2016, Pharmacol Ther 160:65-83). A number of PARP inhibitors are known in the art, such as olaparib, talazoparib (BMN-673), rucaparib, veliparib, niraparib, CEP 9722, MK 4827, BGB-290 (pamiparib), ABT-888, AG014699, BSI-201, CEP-8983, E7016 and 3-aminobenzamide (see, e.g., Rouleau et al., 2010, Nat Rev Cancer 10:293-301, Bao et al., 2015, Oncotarget [Epub ahead of print, Sep. 22, 2015]). PARP inhibitors are known to exhibit synthetic lethality, for example in tumors with mutations in BRCA1/2. Olaparib has received FDA approval for treatment of ovarian cancer patients with mutations in BRCA1 or BRCA2. In addition to olaparib, other FDA-approved PARP inhibitors for ovarian cancer include nirapirib and rucaparib. Talazoparib was recently approved for treatment of breast cancer with germline BRCA mutations and is in phase III trials for hematological malignancies and solid tumors and has reported efficacy in SCLC, ovarian, breast, and prostate cancers (Bitler et al., 2017, Gynecol Oncol 147:695-704). Veliparib is in phase III trials for advanced ovarian cancer, TNBC and NSCLC (see Wikipedia under “PARP_inhibitor”). Not all PARP inhibitors are dependent on BRCA mutation status and niraparib has been approved for maintenance therapy of recurrent platinum sensitive ovarian, fallopian tube or primary peritoneal cancer, independent of BRCA status (Bitler et al., 2017, Gynecol Oncol 147:695-704).

Any such known PARP inhibitor may be utilized in combination with an anti-Trop-2 ADC, such as sacituzumab govitecan or DS-1062. Synthetic lethality and synergistic inhibition of tumor growth has been demonstrated for the combination of sacituzumab govitecan with olaparib, rucaparib and talazoparib in nude mice bearing TNBC xenografts (Cardillo et al., 2017, Clin Cancer Res 23:3405-15). The beneficial effects of combination therapy were observed independently of BRCA1/2 mutation status (Cardillo et al., 2017, Clin Cancer Res 23:3405-15).

CDK12 Inhibitors

Cyclin-dependent kinase 12 (CDK12) is a cell cycle regulator that has been reported to act in concert with PARP inhibitors and knockdown of CDK12 activity was observed to promote sensitivity to olaparib (Bitler et al., 2017, Gynecol Oncol 147:695-704). CDK12 appears to act at least in part by regulating expression of DDR genes (Krajewska et al., 2019, Nature Commun 10:1757). Various inhibitors of CDK12 are known, such as dinaciclib, flavopiridol, roscovitine, THZ1 or THZ531 (Bitler et al., 2017, Gynecol Oncol 147:695-704; Krajewska et al., 2019, Nature Commun 10:1757; Paculova & Kohoutek, 2017, Cell Div 12:7). Combination therapy with PARP inhibitors and dinaciclib reverses resistance to PARP inhibitors (Bitler et al., 2017, Gynecol Oncol 147:695-704). In the subject methods, it may be of use to combine therapy with an anti-Trop-2 ADC with the combination of a PARP inhibitor and a CDK12 inhibitor.

RAD51 Inhibitors

BRCA1 and BRCA2 encode proteins that are essential for the HR DNA repair pathway and mutations in these genes require increased reliance on NHEJ pathways for tumor survival. PARP is a critical protein for NHEJ mediated DNA repair and use of PARP inhibitors (PARPi) in BRCA mutated tumors (e.g., ovarian cancer, TNBC) provides synthetic lethality. However, not all BRCA mutated tumors are sensitive to PARPi and many that are initially sensitive will develop resistance.

RAD51 is another central protein in the HR pathway and is frequently overexpressed in cancer cells (see Wikipedia under “RAD51”). Increased expression of RAD51 may compensate, in part, for BRCA mutations and decrease sensitivity to PARP inhibitors. It has been demonstrated that sacituzumab govitecan, an anti-Trop-2 ADC carrying a topoisomerase I inhibitor, can at least partially compensate for RAD51 overexpression (see U.S. patent application Ser. No. 15/926,537). Thus, a rationale exists for combination therapy using a topoisomerase I-inhibiting ADC with a RAD51 inhibitor, with or without a PARP inhibitor.

Combination therapy with ADCs may utilize any Rad51 inhibitor known in the art, including but not limited to B02 ((E)-3-benzyl-2(2-(pyridin-3-yl)vinyl) quinazolin-4(3H)-one) (Huang & Mazin, 2014, PLoS ONE 9(6):e100993); RI-1 (3-chloro-1-(3,4-dichlorophenyl)-4-(4-morpholinyl)-1H-pyrrole-2,5-dione) (Budke et al., 2012, Nucl Acids Res 40:7347-57); DIDS (4,4′-diisothiocyanostilbene-2,2′-disulfonic acid) (Ishida et al., 2009, Nucl Acids Res 37:3367-76); halenaquinone (Takaku et al., 2011, Genes Cells 16:427-36); CYT-0851 (Cyteir Therapeutics, Inc.), IBR₂ (Ferguson et al., 2018, J Pharm Exp Ther 364:46-54) or imatinib (Choudhury et al., 2009, Mol Cancer Ther 8:203-13). Many of these are available from commercial sources (e.g., B02, Calbiochem; RI-1, Calbiochem; DIDS, Tocris Bioscience; halenaquinone, Angene International Ltd., Hong Kong; imatinib (GLEEVAC®), Novartis).

As discussed above, combination therapy with an ADC and both a RAD51 inhibitor and a PARP inhibitor may be of use for treating cancer.

ATM Inhibitors

ATM and ATR are key mediators of DDR, acting to induce cell cycle arrest and facilitate DNA repair via their downstream targets (Weber & Ryan, 2015, Pharmacol Ther 149:124-38). Many malignant tumors show functional loss or deregulation of key proteins involved in DDR and cell cycle regulation, such as p53, ATM, MRE11, BRCA1/2 or SMC1 (Weber & Ryan, 2015, Pharmacol Ther 149:124-38). As discussed above, defects in certain DDR pathways, such as HRD, may increase reliance of the cancer cell on alternative DDR pathways, thus providing targets for selective inhibition of cancer cells bearing such DDR mutations (Weber & Ryan, 2015, Pharmacol Ther 149:124-38). In addition to the effects of BRCA1/2 mutations on susceptibility to PARP inhibitors, other functional changes in DDR proteins that can increase sensitivity to DNA damaging anti-cancer treatments can include changes in DNA-PKcs (Zhao et al., 2006, Cancer Res 66:5354-62), ATM (Golding et al., 2012, Cell Cycle 11:1167-73), ATR (Fokas et al., 2012, Cell Death Dis 3:e441), CHK1 and CHK2 (Mathews et al., 2007, Cell Cycle 6:104-10; Riesterer et al., 2011, Invest New Drugs 29:514-22). In principle, the effects of such sensitizing mutations may be reproduced by combination therapy using inhibitors against the relevant DDR proteins.

ATM and ATR are members of the phosphatidylinositol 2-kinase-related kinase (PIKK) family, which also includes DNA-PKcs/PRKDC, MTOR/FRAP and SMG1 (Weber & Ryan, 2015, Pharmacol Ther 149:124-38). Due to the high degree of sequence homology between the various PIKK proteins, cross-reactivity is often observed between inhibitors of different PIKK proteins and may result in undesirable toxicities. Use of inhibitors with high affinity for ATM or ATR, compared to other PIKK proteins, is preferred.

ATM attaches to sites of DSBs by binding to the MRN complex (MRE11-RAD50-NBS1) (Weber & Ryan, 2015, Pharmacol Ther 149:124-38). Binding to MRN activates ATM kinase and promotes phosphorylation of its downstream targets—p53, CHK2 and Mdm2—which in turn activates cell cycle checkpoint activity (Weber & Ryan, 2015, Pharmacol Ther 149:124-38). Other downstream effectors of ATM include BRCA1, H2AX and p21 (Ronco et al., 2017, Med Chem Commun 8:295-319). Both the ATM and ATR pathways inhibit activity of CDC25C and CDK1 (Ronco et al., 2017, Med Chem Commun 8:295-319).

Various inhibitors of ATM are known in the art. Caffeine inhibits both ATM and ATR and sensitizes cells to the effects of ionizing radiation (Weber & Ryan, 2015, Pharmacol Ther 149:124-38). Wortmannin is a relatively non-specific inhibitor of PIKK and has activity against ATM, PI3K and DNA-PKcs (Weber & Ryan, 2015, Pharmacol Ther 149:124-38). CP-466722, KU-55933, KU-60019, and KU-59403 are all relatively selective for ATM and have been reported to sensitize cells to the effects of ionizing radiation (Weber & Ryan, 2015, Pharmacol Ther 149:124-38). KU-59403 also increased the anti-tumor efficacy of etoposide and irinotecan, while KU-55933 increased cancer sensitivity to doxorubicin and etoposide (Weber & Ryan, 2015, Pharmacol Ther 149:124-38). The effect of KU-60019 was substantially enhanced in p53 mutant cancer cells, suggesting that p53 mutations might be a biomarker for use of ATM inhibitors. The ATM inhibitor AZD0156 has been used in combination with the PARP inhibitor olaparib (Cruz et al., 2018, Ann Oncol 29:1203-10). AZD0156 in combination with the WEE1 inhibitor AZD1775 produced a synergistic anti-tumor effect in prostate cancer xenografts (Jin et al., Cancer Res Treat [Epub ahead of print Jun. 25, 2019]. Other reported ATM inhibitors include CGK733, NVP-BEZ 235, Torin-2, fluoroquinoline 2 and SJ573017 (Ronco et al., 2017, Med Chem Commun 8:295-319). A significant anti-tumor effect was reported for combination therapy with fluoroquinoline 2 and irinotecan (Ronco et al., 2017, Med Chem Commun 8:295-319).

Although none have yet received FDA approval, ATM inhibitors in clinical trials include AZD1390 (AstraZeneca), Ku-60019 (AstraZeneca), AZD0156 (AstraZeneca)

APR Inhibitors

ATR is another central kinase involved in regulation of DDR. In contrast to ATM, ATR is activated by single-stranded DNA structures (ssDNA), which may occur at resected DSBs or stalled replication forks (Weber & Ryan, 2015, Pharmacol Ther 149:124-38). ATR binds to ATRIP (ATR-interacting protein), which controls localization of ATR to sites of DNA damage (Weber & Ryan, 2015, Pharmacol Ther 149:124-38). ssDNA binds to RPA, which can bind to ATR/ATRIP and also to RAD17/RFC2-5 which in turn promote binding of RAD9-HUS1-RAD1 (9-1-1 complex) onto the DNA ends (Weber & Ryan, 2015, Pharmacol Ther 149:124-38). The 9-1-1 complex recruits TopBP1, which activates ATR (Weber & Ryan, 2015, Pharmacol Ther 149:124-38). ATR then activates CHK1, which promotes DNA repair, stabilization and transient cell cycle arrest (Weber & Ryan, 2015, Pharmacol Ther 149:124-38). Other downstream effectors of ATR function include Cdc25A, Cdc25C, WEE1, Cyclin B and cdc2 (Ronco et al., 2017, Med Chem Commun 8:295-319). The ATM and ATR pathways are partially overlapping and inhibition of one pathway may be partially compensated by activity of the other pathway. In certain embodiments, combination therapy with inhibitors of ATM and ATR, or use of inhibitors that are active against both ATM and ATR, may be preferred. In other embodiments, ATR inhibitors may be indicated for treating cancers where a mutation or other inactivating change inhibits ATM function in the cancer cell.

A number of ATR selective inhibitors have been developed. Schisandrin B is purported to be selective for ATR (Nischida et al., 2009, Nucleic Acids Res 73:5678-89), however with only weak toxicity. More potent inhibitors such as NU6027, BEZ235, ETP46464 and Torin 2 showed cross-reactivity with other PIKK proteins (Weber & Ryan, 2015, Pharmacol Ther 149:124-38). More potent and selective ATR inhibitors have been developed by Vertex Pharmaceuticals, such as VE-821 and VE-822 (aka VX-970, M6620, berzosertib, Merck). Other ATR inhibitors include AZ20 (AstraZeneca), AZD6738 (ceralasertib), M4344 (Merck), (Weber & Ryan, 2015, Pharmacol Ther 149:124-38) as well as EPT-46464 (Brandsma et al., 2017, Expert Opin Investig Drugs 26:1341-55). BAY1895344 (Bayer), BAY-937 (Bayer), AZD6738 (AstraZeneca), BEZ235 (dactolisib), CGK 733 and VX-970 (M6620) are in clinical trials for cancer therapy. AZD6738 was reported to be synthetically lethal with p53 and ATM defects (Ronco et al., 2017, Med Chem Commun 8:295-319).

Combination therapy with VE-821 was shown to enhance sensitivity to cisplatin and gemcitabine in vivo, while AZD6738 significantly increased sensitivity to carboplatin (Weber & Ryan, 2015, Pharmacol Ther 149:124-38). VX970 (M6620) increased sensitivity to a variety of DNA damaging agents, such as cisplatin, oxaliplatin, gemcitabine, etoposide and SN-38 (Weber & Ryan, 2015, Pharmacol Ther 149:124-38). Chemisensitization was more pronounced in cancer cells with p53-deficiency (Weber & Ryan, 2015, Pharmacol Ther 149:124-38). A phase I study of combination therapy with M6620 and topotecan showed improved efficacy in platinum-refractory SCLC, which tends to be non-responsive to topotecan alone (Thomas et al. 2018, J Clin Oncol 36:1594-1602). AZD6738 enhanced sensitivity to carboplatin (Weber & Ryan, 2015, Pharmacol Ther 149:124-38). Various cancer chemotherapeutic agents have been reported to have additive and/or synergistic effects with ATR inhibitors. These include, but are not limited to, gemcitabine, cytarabine, 5-fluorouracil, camptothecin, SN-38, cisplatin, carboplatin and oxaliplatin. [See, e.g., Wagner and Kaufmann, 2010, Pharmaceuticals 3:1311-34] Such agents may be utilized to further enhance combination therapy with anti-Trop-2 ADCs and ATR inhibitors.

CHK1 Inhibitors

CHK1 is a phosphorylation target of the ATR kinase and is a downstream mediator of ATR activity. Phosphorylation of CHK1 by ATR activates CHK1 activity, which in turn phosphorylates Cdc25A and Cdc25C, mediating ATR dependent DNA repair mechanisms (Wagner and Kaufmann, 2010, Pharmaceuticals 3:1311-34).

A variety of CHK1 inhibitors are known in the art, including some that are currently in clinical trials for cancer treatment. Any known CHK1 inhibitor may be utilized in combination with an anti-Trop-2 ADC, including but not limited to XL9844 (Exelixis, Inc.), UCN-01, CHIR-124, AZD7762 (AstraZeneca), AZD1775 (Astrazeneca), XL844, LY2603618 (Eli Lilly), LY2606368 (prexasertib, Eli Lilly), GDC-0425 (Genentech), PD-321852, PF-477736 (Pfizer), CBP501, CCT-244747 (Sareum), CEP-3891 (Cephalon), SAR-020106 (Sareum), Arry-575 (Array), SRA737 (Sareum), V158411 and SCH 900776 (aka MK-8776, Merck). [See Wagner and Kaufmann, 2010, Pharmaceuticals 3:1311-34; Thompson and Eastman, 2013, Br J Clin Pharmacol 76:3; Ronco et al., 2017, Med Chem Commun 8:295-319] CHIR-124 was reported to potentiate the activity of topoisomerase I inhibitors in mouse xenografts (Ronco et al., 2017, Med Chem Commun 8:295-319). CCT244747 showed anti-tumor activity in combination with gemcitabine and irinotecan (Ronco et al., 2017, Med Chem Commun 8:295-319). Clinical trials have been performed with LY2603618 and SCH900776 (Ronco et al., 2017, Med Chem Commun 8:295-319).

CHK2 Inhibitors

Several CHK2 inhibitors are known and may be utilized in combination with an ADC and/or other DDR inhibitors or anti-cancer agents. Such known CHK2 inhibitors include, but are not limited to, NSC205171, PV1019, CI2, CI3 (Gokare et al., 2016, Oncotarget 7:29520-30), 2-arylbenzimidazole (ABI, Johnson & Johnson), NSC109555, VRX0466617 and CCT241533 (Ronco et al., 2017, Med Chem Commun 8:295-319). PV1019 showed enhanced activity in combination with topotecan or camptothecin (Ronco et al., 2017, Med Chem Commun 8:295-319). However, the required dosages were too high to be of therapeutic use (Ronco et al., 2017, Med Chem Commun 8:295-319). Ronco et al. concluded that the CHK2 inhibitors developed to date were significantly less active as anti-cancer agents than CHK1, ATM or ATR inhibitors (Ronco et al., 2017, Med Chem Commun 8:295-319).

WEE1 Inhibitors

WEE1 is overexpressed in many forms of cancer including breast cancer, glioma, glioblastoma, nasopharyngial and drug-resistant cancers (Ronco et al., 2017, Med Chem Commun 8:295-319). WEE1 is a key intermediary in the ATR pathway and is activated by CHK1 (Ronco et al., 2017, Med Chem Commun 8:295-319). WEE1 exerts an inhibitory effect on Cyclin B/cdc2 and CDK1, which in turn regulate cell cycle arrest (Ronco et al., 2017, Med Chem Commun 8:295-319. There are relatively few WEE1 inhibitors available, compared to other components of DDR.

The WEE1 inhibitor AZD1775 (MK1775) has been used in clinical trials in combination with DNA-damaging therapies, such as fludarabine, cisplatin, carboplatin, paclitaxel, gemcitabine, docetaxel, irinotecan or cytarabine (Matheson et al, 2016, Trends Pharm Sci 37:P872-81; see also clinicaltrials.gov). Combination therapy with inhibitors of WEE1 and CHK1/2 is reported to produce a synergistic effect in cancer xenografts (Ronco et al., 2017, Med Chem Commun 8:295-319). Thus, it may be of use to combine therapy with an anti-Trop-2 ADC, an inhibitor of WEE1 and one or more inhibitors of CHK1/2. Other known WEE1 inhibitors include PD0166285 and PD407824. However, these appear to be significantly less clinically useful than MK-1775 (Ronco et al., 2017, Med Chem Commun 8:295-319).

Other DDR Inhibitors

In addition to the major control points discussed above, various inhibitors of other proteins in the DDR pathways have been discovered (Srivastava & Raghavan, 2015, Chem Biol 22:17-29). Due to non-specific interaction and the high degree of homology between various kinases in DDR, some of these inhibitors exhibit cross-reactivity with other DDR proteins.

Mirin is an HR inhibitor that is targeted against MRE11 (Srivastava & Raghavan, 2015, Chem Biol 22:17-29). M1216 and NSC19630 inhibit, respectively, the RecQ helicases BLM and WRN (Srivastava & Raghavan, 2015, Chem Biol 22:17-29). NSC 130813 was developed as an ERCC1 inhibitor, which shows synergistic activity with cisplatin and mitomycin C (Srivastava & Raghavan, 2015, Chem Biol 22:17-29). Among the NHEJ proteins, DNA-PKcs is inhibited by Wortmannin, LY294002, MSC2490484A (M3814), VX-984 (M9831) and NU7026 (Srivastava & Raghavan, 2015, Chem Biol 22:17-29; Brandsma et al., 2017, Expert Opin Investig Drugs 26:1341-55). These and other known DDR inhibitors may be used in combination therapy with an anti-Trop-2 ADC in the subject methods and compositions.

Combination Therapy with ADCs and Other Anti-Cancer Drugs

PI3K/AKT Inhibitors

The phophatidylinositol-3-kinase (PI3K)/AKT pathway is genetically targeted in more tumor types than any other growth factor signaling pathway and is frequently activated as a cancer driver (Guo et al., 2015, J Genet Genomics 42:343-53). There is considerable sequence homology between PI3K and the PI3K-related kinases (PIKK) ATM, ATR and DNA-PK, with frequent cross-reactivity between inhibitors of the different kinases. Inhibitors of PI3K, AKT and PIKK are being actively pursued for cancer therapy (Guo et al., 2015, J Genet Genomics 42:343-53).

In certain embodiments, inhibitors of PI3K and/or the various AKT isoforms (AKT1, AKT2, AKT3) may be utilized in combination therapy with an anti-Trop-2 ADC, alone or in combination with other DDR inhibitors. A variety of PI3K inhibitors are known, such as idelalisib, Wortmannin, demethoxyviridin, perifosine, PX-866, IPI-145 (duvelisib), BAY 80-6946, BEZ235, RP6530, TGR1202, SF1126, INK1117, GDC-0941, GDC-0980, BKM120, XL147, XL765, Palomid 529, GSK1059615, ZSTK474, PWT33597, IC87114, TG100-115, CAL263, PI-103, GNE477, CUDC-907, AEZS-136, NVP-BYL719, NVP-BEZ235, SAR260301, TGR1202 or LY294002. BEZ235, a pan-PI3K inhibitor, was reported to potently kill B-cell lymphomas and human cell lines bearing IG-cMYC translocations (Shortt et al., 2013, Blood 121:2964-74).

AKT is a downstream mediator of PI3K activity. AKT is composed of three isoforms in mammals—AKT1, AKT2 and AKT3 (Guo et al., 2015, J Genet Genomics 42:343-53). The different isoforms have different functions. AKT1 appears to regulate tumor initiation, while AKT2 primarily promotes tumor metastasis (Guo et al., 2015, J Genet Genomics 42:343-53). Following activation by PI3K, AKT phosphorylates a number of downstream effectors that have widespread effects on cell survival, growth, metabolism, tumorigenesis and metastasis (Guo et al., 2015, J Genet Genomics 42:343-53). AKT inhibitors include MK2206, GDC0068 (ipatasertib), AZD5663, ARQ092, BAY1125976, TAS-117, AZD5363, GSK2141795 (uprosertib), GSK690693, GSK2110183 (afuresertib), CCT128930, A-674563, A-443654, AT867, AT13148, triciribine and MSC2363318A (Guo et al., 2015, J Genet Genomics 42:343-53; Xing et al., 2019, Breast Cancer Res 21:78; Nitulescu et al., 2016, Int J Oncol 48:869-85). Any such known AKT inhibitor may be used in combination therapy with anti-Trop-2 ADCs and/or DDR inhibitors. MK-2206 monotherapy showed limited clinical activity in patients with advanced breast cancer who showed mutations in PIK3CA, AKT1 or PTEN and/or PTEN loss (Xing et al., 2019, Breast Cancer Res 21:78). MK-2206 appeared to be more efficacious in combination with paclitaxel to treat breast cancer (Xing et al., 2019, Breast Cancer Res 21:78).

mTOR is a key downstream target of AKT, with global effects on cell metabolism. Inhibitors for mTOR that have been developed for cancer therapy include temsirolimus, everolimus, AZD8055, MLN0128 and OSI-027 (Guo et al., 2015, J Genet Genomics 42:343-53). Such mTOR inhibitors may also be utilized in combination therapy with ADCs and/or DRR inhibitors.

Guo et al. (2015, J Genet Genomics 42:343-53) analyzed genetic alterations in 20 components of the PI3K/AKT pathway, including GNB2LI, EGER, PIK3CA, PIK3R1, PIK3R2, PTEN, PDPK1, AKT1, AKT2, AKT3, FOXO1, FOXO3, MTOR, RICTOR, TSC1, TSC2, RHEB, AK1LSI, RPTOR and MLST8. They observed genetic alterations in every component of the PI3K/AKT pathway in different cancer cells. Genetic alterations were identified in every form of cancer examined, ranging from 6% in thyroid cancer to 95% in endometrioid cancer (Guo et al., 2015, J Genet Genomics 42:343-53). The PIK3CA gene, encoding the pi 10a subunit of PI3K, was found to be the most commonly altered oncogene in cancers in general (Guo et al., 2015, J Genet Genomics 42:343-53). Mutations in PTEN were also common, as was overexpression of RHEB (Guo et al., 2015, J Genet Genomics 42:343-53). Although not commonly mutated, AKT amplification was frequently observed in ovarian, uterine, breast, liver and bladder cancers (Guo et al., 2015, J Genet Genomics 42:343-53). However, AKT3 expression was reported to be downregulated in high-grade serous ovarian cancer (Yeganeh et al., 2017, Genes & Cancer 8:784-98).

CDK4 is a downstream effector of PI3K, in a pathway mediated by protein kinase C. CDK4/6 inhibitors interfere with cell cycle progression and include abemaciclib, palbociclib and ribociclib (Schettini et al., 2018, Front Oncol 12:608).

Other Anti-Cancer Agents

Although the emphasis in the present application is on combinations of anti-Trop-2 ADCs with DDR inhibitors, the subject methods and compositions may include use of one or more other known anti-cancer agents. Any such anti-cancer agent may be used with the subject ADCs, with or without a DDR inhibitor. The various anti-cancer therapeutic agents may be administered concurrently or sequentially. Such agents may include, for example, drugs, toxins, oligonucleotides, immunomodulators, hormones, hormone antagonists, enzymes, enzyme inhibitors, radionuclides, angiogenesis inhibitors, etc. Exemplary anti-cancer agents include, but are not limited to, cytotoxic drugs such as vinca alkaloids, anthracyclines such as doxorubicin, gemcitabine, epipodophyllotoxins, taxanes, antimetabolites, alkylating agents, antibiotics, SN-38, COX-2 inhibitors, antimitotics, anti-angiogenic and pro-apoptotic agents, platinum-based agents, taxol, camptothecins, proteosome inhibitors, mTOR inhibitors, HD AC inhibitors, tyrosine kinase inhibitors, and others. Other useful anti-cancer cytotoxic drugs include nitrogen mustards, alkyl sulfonates, nitrosoureas, triazenes, folic acid analogs, COX-2 inhibitors, antimetabolites, pyrimidine analogs, purine analogs, platinum coordination complexes, mTOR inhibitors, tyrosine kinase inhibitors, proteosome inhibitors, HD AC inhibitors, camptothecins, hormones, and the like. Suitable cytotoxic agents are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications.

Specific drugs of use for combination therapy may include 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxy camptothecin, carmustine, celecoxib, chlorambucil, cisplatin, COX-2 inhibitors, irinotecan (CPT-11), SN-38, carboplatin, cladribine, crizotinib, cyclophosphamide, cytarabine, dacarbazine, dasatinib, dinaciclib, docetaxel, dactinomycin, daunorubicin, DM1, DM3, DM4, doxorubicin, 2-pyrrolinodoxorubicine (2-PDox), cyano-morpholino doxorubicin, doxorubicin glucuronide, endostatin, epirubicin glucuronide, erlotinib, estramustine, epipodophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, famesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, monomethylauristatin F (MMAF), monomethylauristatin D (MMAD), monomethylauristatin E (MMAE), navelbine, neratinib, nilotinib, nitrosourea, olaparib, plicamycin, procarbazine, paclitaxel, PCI-32765, pentostatin, PSI-341, raloxifene, semustine, SN-38, sorafenib, streptozocin, SU11248, sunitinib, tamoxifen, temazolomide, transplatin, thalidomide, thioguanine, thiotepa, teniposide, topotecan, uracil mustard, vatalanib, vinorelbine, vinblastine, vincristine, vinca alkaloids and ZD1839.

Exemplary immunomodulators of use in combination therapy include a cytokine, a lymphokine, a monokine, a stem cell growth factor, a lymphotoxin, a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), TGF-α, TGF-β, insulin-like growth factor (ILGF), erythropoietin, thrombopoietin, tumor necrosis factor (TNF), TNF-α, TNF-β, a mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-α, interferon-β, interferon-γ, interferon-λ S1 factor, IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 and IL-25, LIF, kit-ligand, FLT-3, angiostatin, thrombospondin, endostatin, lymphotoxin, and the like.

These and other known anti-cancer agents may be used in combination with an ADC and/or DDR inhibitor to treat cancer.

Biomarker Detection

Various biomarkers are discussed above, in connection with inhibitors for specific classes of DDR proteins. For example, BRCA mutations are well known to be of use for predicting susceptibility to PARP inhibitors. The use of these and other cancer biomarkers is discussed in more detail below. Such biomarkers may be of use to detect or diagnose various forms of cancer or to predict the efficacy and/or toxicity of ADC monotherapy and/or of combination therapies with ADCs and one or more other anti-cancer agents, such as DDR inhibitors or alternative anti-cancer agents.

A cancer biomarker, as used herein, is a molecular marker associated with malignant cells. Protein biomarkers for cancer have been known and detected since the mid-19^{th} century. For example, Bence Jones proteins were first identified in the urine of multiple myeloma patients in 1846, while prostatic acid phosphatase was detected in the serum of prostate cancer patients as early as 1933 (Virji et al., 1988, CA Cancer J Clin 38:104-26). Numerous other tumor-associated antigens (TAAs) have been detected in various forms of cancer, including but not limited to carbonic anhydrase IX, CCL19, CCL21, CSAp, HER-2/neu, CD1, CD1a, CD5, CD14, CD15, CD19, CD20, CD21, CD22, CD23, CD29, CD30, CD32b, CD33, CD37, CD38, CD40, CD40L, CD44, CD45, CD46, CD52, CD54, CD55, CD59, CD67, CD70, CD74, CD79a, CD83, CD95, CD126, CD133, CD138, CD147, CEACAM5, CEACAM6, alpha-fetoprotein (AFP), VEGF, ED-B fibronectin, EGP-1 (Trop-2), EGP-2, EGF receptor (ErbB1), ErbB2, ErbB3, Factor H, Flt-3, HMGB-1, hypoxia inducible factor (HIF), insulin-like growth factor (ILGF), IL-13R, IL-2, IL-6, IL-8, IL-17, IL-18, IP-10, IGF-1R, HCG, HLA-DR, CD66a-d, MAGE, MCP-1, MIP-1A, MUC5ac, PSA (prostate-specific antigen), PSMA, NCA-95, EpCAM, Le(y), mesothelin, tenascin, Tn antigen, Thomas-Friedenreich antigens, TNF-alpha, TRAIL receptor R1, TRAIL receptor R2, VEGFR, RANTES and various oncogene proteins.

Such protein biomarkers have historically been detected in either biopsy samples of solid tumors, or in biological fluids such as blood or urine (liquid biopsy). Many techniques for protein detection are well known in the art and may be utilized to detect protein biomarkers, such as ELISA, Western blotting, immunohistochemistry, HPLC, mass spectroscopy, protein microarrays, fluorescence microscopy and similar techniques. Many protein-based assays rely on specific protein/antibody interactions for detection. While such assays are of standard use in clinical cancer diagnostics and may be utilized in the subject methods and compositions, the following discussion is more focused on detection of nucleic acid biomarkers for cancer. Preferably, such nucleic acid biomarkers are detected in liquid samples (blood, plasma, serum, lymphatic fluid, urine, cerebrospinal fluid, etc.) from a patient. This is a rapidly evolving field and highly sensitive and specific tests for detecting nucleic acid biomarkers are still being developed. In general, the discussion of liquid biopsy nucleic acid biomarkers below will focus on analysis of cell-free DNA (cfDNA), circulating tumor DNA (ctDNA) or circulating tumor cells (CTCs).

cfDNA Analysis

cfDNA (cell free DNA) refers to extracellular DNA occurring in blood or other body fluids. cfDNA is present primarily in the form of short nucleic acid fragments of about 150 to 180 bp in length that are released from normal or tumor cells by apoptosis and necrosis, or are shed from cells by formation of exosomes or microvesicles (Huang et al., 2019, Cancers 11:E805; Kubiritova et al., 2019, Int J Mol Sci 20:3662). Longer fragment length cfDNA may also be present, and in cancer patients may range up to 10,000 bp in size (Bronkhorst et al., 2019, Biomol Detect Quantif 18:100087). cfDNA levels are typically elevated in cancer patients (Pos et al., 2018, J Immunol 26:937-45) and a fraction of the cfDNA in the plasma of cancer patients is derived from cancer cells (Stroun et al., 1989, Oncology 46:318-22).

It has been proposed that cfDNA may be of wide utility in cancer management, including staging and prognosis, tumor localization, stratification of initial therapy, monitoring therapeutic response, monitoring residual disease and relapse and identifying mechanisms of acquired drug resistance (Bronkhorst et al., 2019, Biomol Detect Quantif 18:100087). The utility of cfDNA in clinical practice has been validated by FDA approval of the COBAS® EGFR Mutation Test v2, designed to identify lung cancer patients eligible for therapy with erlotinib or osimertinib; and EPI PROCOLON®, a colorectal cancer screening test based on the methylation status of the SEPT9 promoter (Bronkhorst et al., 2019, Biomol Detect Quantif 18:100087).

Analysis of cfDNA from a liquid sample may involve preanalytical separation, concentration and purification. While these may be performed manually, several automated systems or kits for extracting cfDNA from liquid samples are available and may be preferably utilized. These include the NUCLEOMAG® DNA Plasma kit (Takara), MAGMAX™ Cell-Free DNA Isolation kit for use with the KINGFISHER™ instrument (ThermoFisher), the Omega Bio-tek automated system for use with the Hamilton MICROLAB® STAR™ platform, the MAXWELL® RSC (MR) cfDNA Plasma Kit, and numerous others. Such methods and apparatus for isolation of cfDNA from liquid samples are well known in the art and any such known method or apparatus may be used in the practice of the subject methods.

Once isolated, cfDNA may be analyzed for the presence of biomarkers. Traditional methods have been used to detect DNA mutations, insertions, deletions, recombinations or other biomarkers, such as Sanger dideoxy sequencing (manually or by Applied Biosystems workstation), RT-PCR, fluorescence microscopy, SNP hybridization, GENECHIP® and other known techniques. Where specific mutational “hot spots” are known and well characterized, PCR-based analysis can be used for biomarker detection. For example, Qiagen sells a PI3K Mutation Test Kit to detect 4 mutations (H1047R, E542K, E545D, E545K) in exons 9 and 20 of the PI3K oncogene, using ARMS® and SCORPION® technology. Detection of 1% mutant sequences in a background of wild-type genomic DNA is possible. BRCANALYSISCDX® (Myriad) is another PCR based test to detect mutations in BRCA1 or BRCA2. Other tests designed to detect biomarkers in specific genes or panels of genes are commercially available.

While these are sufficient to detect a limited number of nucleic acid biomarkers that are well characterized and known to be associated with specific types of cancers, a more global approach to detection of a panoply of biomarkers, which may occur in multiple locations or which are heterogenous or poorly characterized, requires use of a more advanced DNA analytical technique, such as next generation sequencing, discussed below (Kubiritova et al., 2019, Int J Mol Sci 20:3662). NGS techniques of use with liquid biopsy samples have been reviewed (e.g., Chen & Zhao, 2019, Human Genomics 13:34).

Next generation sequencing (NGS) may be directed towards coding regions of DNA (whole exome sequencing) or to both coding and non-coding regions (whole-genome sequencing). The analysis of cancer biomarkers is generally more concerned with coding region variation and regulatory sequences, such as promoters. Specific target gene panels may also be optimized for NGS (Johnson et al., 2013, Blood 122:3268-75). There are many variations of NGS techniques and apparatus in use. The following discussion is a generalized discussion of some common features of NGS.

After obtaining a sample of, for example, cfDNA, the initial step in NGS is to cut genomic DNA or cDNA into short fragments of a few hundred basepairs, which is the average size of cfDNA. If longer DNA sequences are present, they may need to be fragmented to appropriate size. Short oligonucleotide linkers (adaptors) may be added to the DNA fragments. If multiple samples are to be analyzed simultaneously, the linkers may be labeled with unique fluorescent or other detectable probes (molecular barcodes) to allow assignment of sequences to different individuals or to different genes. Linkers also allow for PCR amplification if the source DNA is too limited for signal detection. Barcode technology may also be used, as discussed below, to identify specific nucleic acid sequences against a background of numerous other nucleic acid species.

The short DNA fragments are converted to single stranded DNA and hybridized to complementary oligonucleotides located in channels on a microscope slide or another type of microfluidic chip apparatus, although other types of solid surfaces may be used. The location of hybridized fragments may detected, e.g. by fluorescence microscopy (Johnson et al., 2013, Blood 122:3268-75). Because the location and sequence of the complementary oligonucleotides are known, the corresponding sequence of the hybridizing DNA fragments may be identified. In various embodiments, the complementary oligonucleotides may serve as primers for further extension by DNA polymerase activity to generate additional sequence data.

In the Illumina NGS system, complementary DNA attached to primers on the surface of a flow cell is replicated to form small clusters of identical DNA sequence for signal amplification. Unlabeled dNTPs and DNA polymerase are added to lengthen and join the attached strands of DNA to make “bridges” of dsDNA between the primers on the flow cell. The dsDNA is then broken down into ssDNA. Primers and fluorescently labeled terminators that are specific for each of the four nucleotides are added. Once a nucleotide is incorporated in a growing chain, further elongation is blocked until the terminator is removed. Fluorescence microscopy is used to identify which nucleotide has been incorporated at each location of the flow cell. The terminators are removed and the next round of polymerization proceeds. The individual short (about 150 bp) sequences may be compiled into larger exonic or non-coding genomic sequences.

The Illumina platform is exemplary only and many other NGS systems are available, each of which uses some variations in the techniques, chemistries and protocols used to obtain nucleic acid sequences (see, e.g., Besser et al., 2018, Clin Microbiol Infect. 24:335-41). Other common detection platforms may involve pyrosequencing (based on pyrophosphate release) (see, e.g., Jouini et al., 2019, Heliyon 19:e01330) or ION TORRENT™ NGS (based on release of hydrogen ions when a DNTP is incorporated) (see, e.g., Fan et al., 2019, Oncol Rep 42:1580-88).

ctDNA Analysis

ctDNA is cell free DNA that originates in tumor cells. Typically a small fraction of cfDNA, ctDNA may be 0.1% or less of cfDNA in individuals with early stage cancer (Huang et al., 2019, Cancers 11:E805), although estimates of ctDNA frequency as high as 90% of cfDNA have been reported (Volik et al., 2016, Mol Cancer Res 14:898-908). Because of its slightly different size range, ctDNA may be partially enriched from cfDNA by polyacrylamide gel electrophoresis, followed by excision and elution of the appropriate size range (Huang et al., 2019, Cancers 11:E805). However, although such techniques may enrich for ctDNA, the majority of cfDNA at least in early stage cancer will still come from normal cells, resulting in a high signal-to-noise background. The analysis of ctDNA is also complicated by tumor heterogeneity. Techniques have been developed to deal with the low incidence of ctDNA, including droplet digital PCR (ddPCR) and molecular index-based next generation sequencing (Volik et al., 2016, Mol Cancer Res 14:898-908; Wood-Bouwens et al., 2017, J Mol Diagn 19:697-710).

Initial studies of ctDNA relied on real-time allele-specific PCR to detect mutations of interest (Yi et al., 2017, Int J Cancer 140:2642-47). The technique was designed to detect mutations that were only present in cancer cells. However, the sensitivity and specificity of the technique limited its use primarily to individuals with high tumor burden. Digital PCR has increased sensitivity and specificity by limiting dilution of DNA samples, so that individual DNA molecules are present in water-oil emulsion droplets or chambers (Yi et al., 2017, Int J Cancer 140:2642-47). Primers and probes designed to distinguish between mutant and normal alleles of specific genes may be used for amplification and to quantify mutant allele frequency. However, such techniques require prior knowledge of the nucleic acid biomarker to be detected.

Next generation sequencing, particularly massive parallel sequencing, has been applied to ctDNA as well as cfDNA. These methods and systems are discussed in detail in the preceding section. As discussed above, because of the size overlap between cfDNA of normal cells and ctDNA, separation of ctDNA from a much higher concentration of cfDNA is technically difficult. Therefore, analysis of ctDNA has frequently attempted to detect tumor-specific nucleic acid biomarkers against a high background of cfDNA, using the same analytic techniques discussed above.

An interesting variation on this approach utilized capture-based next generation sequencing to detect ALK (anaplastic lymphoma kinase) rearrangement in NSCLC (Wang et al., 2016, Oncotarget 7:65208-17). A capture-based sequencing panel (Burning Rock Biotech Ltd, Guangzhou China) targeting 168 genes and spanning 160 kb of human genomic DNA sequence was used. cfDNA was hybridized with capture probes, separated by magnetic bead binding and then PCR amplified. The amplified samples were sequenced on a NextSeq 500 system (Illumina). Given the difficulties with sizing-based separation techniques, use of capture techniques may be superior for separation of ctDNA from cfDNA. However, this requires targeted analysis of specific sets of genes or prior knowledge of nucleic acid sequence variants present in the tumor cells.

A growing number of studies have examined cancer biomarkers based on ctDNA analysis. Angus et al. (Mol Oncol 2019 13:2361-74) analyzed ctDNA of metastatic colorectal cancer (mCRC) patients by NGS for mutations in RAS and BRAE. Patients with mCRC harboring RAS or BRAF mutations do not respond to anti-EGFR antibodies, such as cetuximah and panitumumab (Angus et al., 2019 13:2361-74). Despite selection of patients for anti-EGFR therapy based on RAS mutations, less than 50% of patients with wild-type mCRC show clinical benefit (Angus et al., 2019 13:2361-74). ctDNA analysis of plasma samples demonstrated heterogeneity in RAS and BRAF mutations in patients identified as wild-type RAS by tumor biopsy. Relative to patients without mutations, those with RAS/BRAF mutations had shorter progression-free survival (1.8 vs. 4.9 months) and overall survival (3.1 vs. 9.4 months) (Angus et al., 2019 13:2361-74). It was concluded that RAS and BRAF mutations in cfDNA/ctDNA are predictive of outcome of cetuximab monotherapy (Angus et al., 2019 13:2361-74).

Galbiati et al. (2019, Cells 8:769) used a combination of microarray probe hybridization with droplet digital PCR (ddPCR) to detect specific mutations in KRAS, NBAS and BRAF and to determine the fractional abundance of the mutant alleles in ctDNA of mCRC patients. The microarray capture probes were specific for KRAS (G12A, G12C, G12D, G12R, G12S, G12V, G13D, Q61H(A>C), Q61H(A>T), Q61K, Q61L, Q61R, A146T), NRAS (G12A, G12C, G12D, G12S, G12V, G13D, G13V) and BRAF (V600E), as well as wild-type sequences (Galbiati et al., 2019, Cells 8:769). After allele-specific hybridization, ssPCR-reporter hybrids were used for detection. ddPCR was performed with the QX100™ DROPLET DIGITAL™ PCR system (Bio-Rad) following microarray analysis. Comparison of the microarray results with tissue biopsy analysis showed an overall concordance of 95%, with two additional KRAS mutations observed that were not found on tissue biopsy (Galbiati et al., 2019, Cells 8:769). It was concluded that ctDNA analysis could be used for non-invasive biomarker detection to guide anti-EGFR antibody therapy in mCRC (Galbiati et al., 2019, Cells 8:769).

These and many other reported studies on cfDNA or ctDNA analysis demonstrate the utility of circulating nucleic acids for detection, prognosis, monitoring response to disease and predicting responsiveness to specific anti-cancer agents and/or combination therapies. It should be noted that, in general, studies of ctDNA have not separated the tumor-derived nucleic acids from normal cell cfDNA, rather the analysis of ctDNA is based on the detection of tumor-specific or tumor-selective markers. The distinction between analysis of cfDNA and ctDNA in cancer diagnostics is therefore somewhat semantic in nature, and all of the techniques, methods and apparatus described in the preceding section on cfDNA may also be used for analysis of ctDNA.

Analysis of Circulating Tumor Cells (CTCs)

It has been proposed that early in tumor progression, cancer cells may be found in low concentration in the circulation (see, e.g., Krishnamurthy et al., 2013, Cancer Medicine 2:226-33; Alix-Panabieres & Pantel, 2013, Clin Chem 50:110-18; Wang et al., 2015, Int J Clin Oncol, 20:878-90). Due to the relatively non-invasive nature of blood sample collection, there has been great interest in the isolation and detection of CTCs, to promote cancer diagnosis at an earlier stage of the disease and as a predictor for tumor progression, disease prognosis and/or responsiveness to drug therapy (see, e.g., Alix-Panabieres & Pantel, 2013, Clin Chem 50:110-18; Winer-Jones et al., 2014, PLoS One 9:e86717; U.S. Patent Appl. Publ. No. 2014/0357659).

Various techniques and apparatus have been developed to isolate and/or detect circulating tumor cells. Several reviews of the field have recently been published (see, e.g., Alix-Panabieres & Pantel, 2013, Clin Chem 50:110-18; Joosse et al., 2014, EMBO Mol Med 7:1-11; Truini et al., 2014, Fron Oncol 4:242). The techniques have involved enrichment and/or isolation of CTCs, generally using capture antibodies against an antigen expressed on tumor cells, and separation with magnetic nanoparticles, microfluidic devices, filtration, magnetic separation, centrifugation, flow cytometry and/or cell sorting devices (e.g., Krishnamurthy et al., 2013, Cancer Medicine 2:226-33; Alix-Panabieres & Pantel, 2013, Clin Chem 50:110-18; Joosse et al., 2014, EMBO Mol Med 7:1-11; Truini et al., 2014, Fron Oncol 4:242; Powell et al., 2012, PLoS ONE 7:e33788; Winer-Jones et al., 2014, PLoS One 9:e86717; Gupta et al., 2012, Biomicrofluidics 6:24133; Saucedo-Zeni et al., 2012, Int J Oncol 41:1241-50; Harb et al., 2013, Transl Oncol 6:528-38). The enriched or isolated CTCs may then be analyzed using a variety of known methods, as discussed further below.

Systems or apparatus that have been used for CTC isolation and detection include the CELLSEARCH® system (e.g., Truini et al., 2014, Front Oncol 4:242), MagSweeper device (e.g., Powell et al., 2012, PLoS ONE 7:e33788), LIQUIDBIOPSY® system (Winer-Jones et al., 2014, PLoS One 9:e86717), APOSTREAM® system (e.g., Gupta et al., 2012, Biomicrofluidics 6:24133), GILUPI CELLCOLLECTOR™ (e.g., Saucedo-Zeni et al., 2012, Int J Oncol 41:1241-50), and ISOFLUX™ system (Harb et al., 2013, Transl Oncol 6:528-38).

To date, the only FDA-approved technology for CTC detection involves the CELLSEARCH® platform (Veridex LLC, Raritan, N.J.), which utilizes anti-EpCAM antibodies attached to magnetic nanoparticles to capture CTCs. Detection of bound cells occurs with fluorescent-labeled antibodies against cytokeratin (CK) and CD45. Fluorescently labeled cells bound to magnetic particles are separated out using a strong magnetic field and are counted by digital fluorescence microscopy. The CELLSEARCH® system has received FDA approval for detection of metastatic breast, prostate and colorectal cancers.

Most CTC detection systems have focused on use of anti-EpCAM capture antibodies (see, e.g., Truini et al., 2014, Front Oncol 4:242; Powell et al., 2012, PLoS ONE 7:e33788; Alix-Panabieres & Pantel, 2013, Clin Chem 50:110-18; Lin et al., 2013, Biosens Bioelectron 40:63-67; Magbanua et al., 2015, Clin Cancer Res 21:1098-105; Harb et al., 2013, Transl Oncol 6:528-38). However, not all metastatic tumors express EpCAM (see, e.g., Mikolajcyzyk et al., 2011, J Oncol 2011:252361; Pecot et al., 2011, Cancer Discovery 1:580-86; Gupta et al., 2012, Biomicrofluidics 6:24133). Attempts have been made to utilize alternative schemes for isolating and detecting EpCAM-negative CTCs, such as use of antibody combinations against TAAs. Antibodies against as many as 10 different TAAs have been utilized in an attempt to increase recovery of metastatic circulating tumor cells (e.g., Mikolajcyzyk et al., 2011, J Oncol 2011:252361; Pecot et al., 2011, Cancer Discovery 1:580-86; Krishnamurthy et al., 2013, Cancer Medicine 2:226-33; Winer-Jones et al., 2014, PLoS One 9:e86717).

The present methods for CTC analysis may be used with an affinity-based enrichment step or without an enrichment step, such as MAINTRAC® (Pachmann et al. 2005, Breast Cancer Res, 7: R975). Methods that use a magnetic device for affinity-based enrichment, include the CELLSEARCH® system (Veridex), the LIQUIDBIOPSY® platform (Cynvenio Biosystems) and the MagSweeper device (Talasaz et al, PNAS, 2009, 106: 3970). Methods that do not use a magnetic device for affinity-based enrichment, include a variety of fabricated microfluidic devices, such as CTC-chips (Stott et al. 2010, Sci Transl Med, 2: 25ra23), HB-chips (Stott et al, 2010, PNAS, 107: 18392), NanoVelcro chips (Lu et al., 2013, Methods, 64: 144), GEDI microdevice (Kirby et al., 2012, PLoS ONE, 7: e35976), and Biocept's ONCOCEE™ technology (Pecot et al., 2011, Cancer Discov, 1: 580).

Use of the FDA-approved CELLSEARCH® system for CTC detection in non-small cell and small cell lung cancer patients is discussed in Truini et al. (2014, Front Oncol 4:242). A 7.5 ml sample of peripheral blood is mixed with magnetic iron nanoparticles coated with an anti-EpCAM antibody. A strong magnetic field is used to separate EpCAM positive from EpCAM-negative cells. Detection of bound CTCs was performed using fluorescently labeled anti-CK and anti-CD45 antibodies, along with DAPI (4′,6′diamidino-2-phenylindole) fluorescent labeling of cell nuclei. CTCs were identified by fluorescent detection as CK positive, CD45 negative and DAPI positive.

The VERIFAST™ system was used for diagnosis and pharmacodynamic analysis of circulating tumor cells (CTCs) in non-small cell lung cancer (NSCLC) (Casavant et al., 2013, Lab Chip 13:391-6; 2014, Lab Chip 14:99-105). The VERIFAST™ platform utilizes the relative dominance of surface tension over gravity in the microscale to load immiscible phases side by side. This pins aqueous and oil fields in adjacent chambers to create a virtual filter between two aqueous wells (Casavant et al., 2013, Lab Chip 13:391-6). Using paramagnetic particles (PMPs) with attached antibody or other targeting moieties, specific cell populations can be targeted and isolated from complex backgrounds through a simple traverse of the oil barrier. In the NSCLC example, streptavidin was conjugated to DYNABEADS® FLOWCOMP™ PMPs (Life Technologies, USA) and cells were captured using biotinylated anti-EpCAM antibody. A handheld magnet was used to transfer CTCs bound to PMPs between aqueous chambers. Collected CTCs were released with PMP release buffer (DYNABEADS®) and stained for EpCAM, EGFR or transcription termination factor (TTF-1). The VERIFAST™ platform integrates a microporous membrane into an aqueous chamber to enable multiple fluid transfers without the need for cell transfer or centrifugation. With physical characteristic scales enabling high precision relative to macroscale techniques, such microfluidic techniques are well adapted to capture and assess CTCs with minimal sample loss. The VERIFAST™ platform effectively captured CTCs from blood of NSCLC patients (Casavant et al., 2013, Lab Chip 13:391-6; 2014, Lab Chip 14:99-105).

The GILUPI CELLCOLLECTOR™ (Saucedo-Zeni et al., 2012, Int J Oncol 41:1241-50) is based on a functionalized medical Seldinger guidewire (FSMW) coated with chimeric anti-EpCAM antibody. The guidewire was functionalized with a polycarboxylate hydrogel layer that was activated with EDC and NHS, allowing covalent bonding of antibody. The antibody-coated FSMW was inserted in the cubital veins of breast cancer or NSCLC lung cancer patients through a standard venous cannula for 30 minutes. Following binding of cells to the guidewire, CTCs were identified by immunocytochemical staining of EpCAM and/or cytokeratins and nuclear staining. Fluorescent labeling was analyzed with an Axio Imager.A1m microscope (Zeiss, Jena, Germany). The FSMW system was capable of enriching EpCAM-positive CTCs from 22 of 24 patients tested, including those with early stage cancer in which distant metastases had not yet been diagnosed. No CTCs were detected in healthy volunteers. An advantage of the FSMW system is that it is not limited by the volume of ex vivo blood samples that may be processed using alternative methodologies. Estimated blood volume in contact with the FSMW during the 30 minute exposure was 1.5 to 3 liters.

These and other methods for CTC isolation may be used to obtain samples for biomarker analysis. Although EpCAM is the most commonly used target for capture antibodies, the various devices may also be used with a different capture antibody, such as an anti-Trop-2 antibody. As the cancer types to be targeted with the ADC combination therapies disclosed herein will generally have high expression of Trop-2, such antibodies may be more efficient for capturing CTCs in patients with such cancers. It is not precluded that the same antibody (e.g., hRS7) might be used both for capture and characterization of CTCs and for treating the underlying tumor, in the form of topoisomerase I inhibitor-conjugated ADCs.

Once CTCs have been isolated from the circulation, they may be analyzed for the presence of biomarkers using standard methodologies disclosed elsewhere herein, for example by PCR, RT-PCR, fluorescence microscopy, ELISA, Western blotting, immunohistochemistry, microfluidic chip technologies, SNP hybridization, molecular barcode analysis or next generation sequencing. Kwan et al. (2018, Cancer Discov 8:1286-99) performed digital analysis of RNA from CTCs in breast cancer. Chemotherapy resistance was associated with ESR1 mutations (L536R, Y537C, Y537N, Y537S, D538G), elevated CTC score and persistent CTC signal after 4 weeks of treatment (Kwan et al., 2018, Cancer Discov 8:1286-99). Rapid tumor progression was associated with biomarkers for PIP, SERPINA3, AGR2, SCGB2A1, EFHD1 and WFDC2.

Shaw et al. (2017, Clin Cancer Res 23:88-96) performed analysis of cfDNA and single CTCs in metastatic breast cancer patients. CTCs were obtained with the CELLSEARCH® apparatus using anti-EpCAM antibodies. Analysis was performed by next generation sequencing of about 2200 mutations in 50 cancer genes. Mutational heterogeneity between individual CTCs was observed in PIK3CA, TP53, ESR1 and KRAS (Shaw et al., 2017, Clin Cancer Res 23:88-96). The cfDNA profiles correlated with those obtained from CTCs (Shaw et al., 2017, Clin Cancer Res 23:88-96). ESR1 and KRAS mutations seen in CTCs were not observed in the primary tumor samples and it was suggested they represent a sub-clonal population of cells or else were acquired with disease progression (Shaw et al., 2017, Clin Cancer Res 23:88-96).

Other Techniques for Biomarker Detection

Detection of nucleic acid biomarkers is not limited to any specific technique or type of molecule or cell. In other embodiments, biomarkers may be in the form of RNA, for example. RNA samples may be obtained from circulation, although they are typically present in very low concentration due to endogenous ribonuclease activity. Alternatively, mRNA may be extracted from solid biopsy samples using standard techniques (see, e.g., Singh et al., 2018, J Biol Methods 5:e95).

Automated systems for detecting RNA biomarkers are commercially available. One such system is the NanoString NCOUNTER® technology. If sufficient RNA is present in a sample, solution phase hybridization of the mRNA occurs with capture probes and fluorescent barcode-labeled reporter probes. The sequences of reporter probes are designed to hybridize to specific nucleic acid biomarkers of interest. Following removal of unhybridized material, the hybridized probes are immobilized and aligned on the surface of a cartridge. The barcode-labeled mRNA is then identified by fluorescent detection of the localized barcodes. The NCOUNTER® system allows simultaneous detection of up to 800 selected nucleic acid targets. Although direct detection of circulating or solid biopsy RNA is preferred, if the sample size is insufficient an RT-PCT step may be added. This inherently reduces the accuracy of the technique, due to amplification bias or other errors that may occur. Direct detection is preferred where reliable quantification is desired, such as determining gene expression levels of various biomarker genes. The NanoString technology may also be used to analyze cfDNA or ctDNA samples.

Souza et al. (2019, J Oncol 8393769) used the NanoString NCOUNTER® Human v3 miRNA Expression panel to analyze circulating cell-free microRNAs in the serum of breast cancer patients. Out of 800 microRNA probes analyzed, 42 showed the presence of significant differentially expressed circulating microRNAs in breast cancer patients and further showed differential expression in different subtypes of breast cancer (Souza et al., 2019, J Oncol 8393769). The biomarker miR-2503p showed the highest correlation with TNBC. It was concluded that liquid biopsy of circulating microRNAs could be suitable for early detection of breast cancer (Souza et al., 2019, J Oncol 8393769).

Another platform for detection of nucleic acid biomarkers is the Affymetrix GENECHIP®. The system can be used with a variety of GENECHIP® microarrays that are preloaded with hybridization probes for RNA or DNA analysis. The probe sets may be custom designed or may be selected from standard chips for SNP detection and can contain up to a million probes per chip (Dalma-Weiszhausz et al., 2006, Methods Enzymol 410:3-28). Different chips have been designed for genomic SNP detection, whole genome expression profiling, whole genome sequencing, differential splice variation and numerous other applications. For example, the Affymetrix Genome-Wide Human SNP Array 6.0 contains 1.8 million genetic markers, including 906,600 SNPs and more than 946,000 probes for detection of copy number variation. The Agilent miRNA Microarray Human Release 12.0 can assay for the presence of 866 miRNA species. The Affymetrix GENECHIP® Human Genome U133 Plus 2.0 Array can analyze the expression of more than 47,000 transcripts, including 38,500 well characterized genes.

DNA methylation may be assayed using standard techniques and apparatus. For example, information on genome-wide DNA methylation may be obtained using the INFINIUM® HumanMethylation450 dataset of The Cancer Genome Atlas (TCGA). Methylation may be detected using the INFINIUM® MethylationEpic Beadchip Kit (Illumina) or INFINIUM® 450K Methylation arrays (Illumina). Alternatively, methylation can be detected using the GOLDENGATE® Assay for Methylation and BEAD ARRAY™ Technology. The Illumina INFINIUM® HD Beadchip can assay almost 1.2 million genomic loci for genotyping and copy number variation. These and many other standard platforms or systems are well known in the art for detecting and identifying cancer bio markers.

Biomarkers for Anti-Cancer Efficacy and/or Toxicity

Numerous cancer biomarkers have been listed above, such as mutations in NBAS, KRAS, BRCA1, BRCA2, p53, ATM, MRE11, SMC1, DNA-PKcs, PI3K, or BRAE. Genes (or their encoded proteins) of interest for biomarker analysis include, but are not limited to, 53BP1, AKT1, AKT2, AKT3, APE1, ATM, ATP BARD1, BAP1, BLM, BRAF, BRCA1, BRCA2, BRIP1 (FANCJ), CCND1, CCNE1, CDKN1, CDK12, CHEK1, CHEK2, CK-19, CSA, CSB, DCLRE1C, DNA2, DSS1, EEPD1, EFHD1, EpCAM, ERCC1, ESR1, EXO1, FAAP24, FANC1, FANCA, FANCC, FANCD1, FANCD2, FANCE, FANCF, FANCM, HER2, HMBS, HR23B, KRT19, KU70, KU80, hMAM, MAGEA1, MAGEA3, MAPK, MGP, MLH1, MRE11, MRN, MSH2, MSH3, MSH6, MUC16, NBM, NBS1, HER, NF-κB, P53, PALB2, PARP1, PARP2, PIK3CA, PMS2, PTEN, RAD23B, RAD50, RAD51, RAD51AP1, RAD51C, RAD51D, RAD52, RAD54, RAF, K-ras, H-ras, N-ras, RBBP8, c-myc, RIF1, RPA1, SCGB2A2, SLFN11, SLX1, SLX4, TMPRSS4, TP53, PROP-2, USP11, VEGF, WEE1, WRN, XAB2, XLF, XPA, XPC, XPD, XPF, XPG, XRCC4 and XRCC7. As discussed in Example 1 below, in certain embodiments genes of interest for biomarker detection may include BRCA1, BRCA2, CHEK2, MSH2, MSH6, TP53, CDKN1A, BAG6, BRSK2, ERN1, FHIT, HIPK2, LGALS12, ZNF622, AEN SART1, USP28, GADD45B, TGFB1, NDRG1, WEE1, PPP1R15A, MYBBP1A, SIRT1, ABL1, HRAS, ZNF385B, POLR2K or DDB2.

In some embodiments genes of interest for biomarker detection comprise BRCA1, BRCA2, CHEK2, MSH2, MSH6, TP53, CDKN1A, BAG6, BRSK2, ERN1, FHIT, HIPK2, LGALS12, ZNF622, AEN SART1, USP28, GADD45B, TGFB1, NDRG1, WEE1, PPP1R15A, MYBBP1A, SIRT1, ABL1, HRAS, ZNF385B, POLR2K and DDB2.

In some embodiments genes of interest for biomarker detection consist of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NDRG1, WEE1, PPP1R15A, MYBBP1A, SIRT1, ABL1, HRAS, ZNF385B, POLR2K and DDB2.

In some embodiments genes of interest for biomarker detection comprise AEN MSH2, MYBBP1A, SART1, SIRT1, USP28, CDKN1A, ABL1, TP53, BAG6, BRCA1, BRCA2, BRSK2, CHEK2, ERN1, FHIT, HIPK2, HRAS, LGALS12, MSH6, ZNF385B, and ZNF622.

In some embodiments genes of interest for biomarker detection consist of AEN MSH2, MYBBP1A, SART1, SIRT1, USP28, CDKN1A, ABL1, TP53, BAG6, BRCA1, BRCA2, BRSK2, CHEK2, ERN1, FHIT, HIPK2, HRAS, LGALS12, MSH6, ZNF385B, and ZNF622.

In some embodiments genes of interest for biomarker detection comprise BRCA1, BRCA2, CHEK2, MSH2, MSH6, TP53, CDKN1A, BAG6, BRSK2, ERN1, FHIT, HIPK2, LGALS12, ZNF622, AEN, SART1, and USP28.

In some embodiments genes of interest for biomarker detection consist of BRCA1, BRCA2, CHEK2, MSH2, MSH6, TP53, CDKN1A, BAG6, BRSK2, ERN1, FHIT, HIPK2, LGALS12, ZNF622, AEN, SART1, and USP28.

In some embodiments genes of interest for biomarker detection comprise POLR2K, DDB2, GADD45B, WEE1, TGFB1, NDRG1, and PPP1R15A.

In some embodiments genes of interest for biomarker detection consist of POLR2K, DDB2, GADD45B, WEE1, TGFB1, NDRG1, and PPP1R15A.

In some embodiments genes of interest for biomarker detection comprise BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NRG1, WEE1, and PPP1R15A.

In some embodiments genes of interest for biomarker detection consist of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NRG1, WEE1, and PPP1R15A.

In some embodiments the biomarker is a plurality of single nucleotide polymorphisms that result in a substitution comprising E155K in ABL1, G706S in ABL1, V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, G12V in HRAS, A278V in LGALS12, N127S in MSH2, S625F in MSH6, H680Y in MYBBP1A, R373Q in SART1, E113Q in SIRT1, *394S in TP53, R282G in TP53, T377P in in TP53, E271K in TP53, Y220C in TP53, E180* in TP53, I987L in USP28, R370Q in ZNF385B and A437E in ZNF622.

In some embodiments the biomarker is a plurality of single nucleotide polymorphisms that result in a substitution consisting of E155K in ABL1, G706S in ABL1, V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, G12V in HRAS, A278V in LGALS12, N127S in MSH2, S625F in MSH6, H680Y in MYBBP1A, R373Q in SART1, E113Q in SIRT1, *394S in TP53, R282G in TP53, T377P in in TP53, E271K in TP53, Y220C in TP53, E180* in TP53, I987L in USP28, R370Q in ZNF385B and A437E in ZNF622.

In some embodiments the biomarker is a plurality of single nucleotide polymorphisms that result in a substitution comprising V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, N127S in MSH2, S625F in MSH6, R373Q in SART1, *394S in TP53, R282G in TP53, T377P in in TP53, E271K in TP53, Y220C in TP53, E180* in TP53, and I987L in USP28.

In some embodiments the biomarker is a plurality of single nucleotide polymorphisms that result in a substitution consisting of V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, N127S in MSH2, S625F in MSH6, R373Q in SART1, *394S in TP53, R282G in TP53, T377P in in TP53, E271K in TP53, Y220C in TP53, E180* in TP53, and I987L in USP28.

In some embodiments the biomarker is a frameshift mutation selected from the group consisting of K1110fs in BAG6, R32fs in CDKN1A, DC33fs in CDKN1A and EG60fs in CDKN1A.

In some embodiments the biomarker is a plurality of frameshift mutations comprising K1110fs in BAG6, R32fs in CDKN1A, DC33fs in CDKN1A, and EG60fs in CDKN1A.

In some embodiments the biomarker is a plurality of frameshift mutations consisting of K1110fs in BAG6, R32fs in CDKN1A, DC33fs in CDKN1A, and EG60fs in CDKN1A.

In some embodiments the biomarker is an increase or decrease in gene expression in the cancer compared to corresponding normal tissue for a gene selected from the group consisting of POLR2K, DDB2, GADD45B, WEE1, TGFB1, NDRG1 and PPP1R15A.

In some embodiments the biomarker is a plurality of increases or decreases in gene expression in the cancer compared to corresponding normal tissue comprising POLR2K, DDB2, GADD45B, WEE1, TGFB1, NDRG1 and PPP1R15A.

In some embodiments the biomarker is a plurality of increases or decreases in gene expression in the cancer compared to corresponding normal tissue consisting of POLR2K, DDB2, GADD45B, WEE1, TGFB1, NDRG1 and PPP1R15A.

In some embodiments the gene is selected from the group consisting of BRCA1, BRCA2, PTEN, ERCC1 and ATM.

In some embodiments the one or more biomarkers comprise or consist of BRCA1, BRCA2, PTEN, ERCC1 and ATM.

Biomarkers of use may come in a variety of forms, such as mutations, insertions, deletions, gene amplification, duplication or rearrangement, promoter methylation, RNA splice variants, SNPs, increased or decreased levels of specific mRNAs or proteins and any other form of biomolecule variation. A number of cancer biomarkers have been identified in the literature, some with predictive value for determining which monotherapy or combination therapy is likely to be effective in a given cancer. Any such known biomarker may be used in the subject methods. The text below summarizes various biomarkers that have been identified to be of use in cancer diagnostics. However, the subject methods are not limited to the specific biomarkers disclosed herein, but may include any biomarkers known in the art.

Biomarkers for Use of Topoisomerase I Inhibitors

Biomarkers for cancer cell sensitivity to or toxicity of inhibitors of topoisomerase I may be correlated with sensitivity to or toxicity of topoisomerase I-inhibiting ADCs, such as sacituzumab govitecan or DS-1062. Cecchin et al. (2009, J Clin Oncol 27:2457-65) examined the predictive value of haplotypes in UGT1A1, UGT1A7 and UGT1A9 in metastatic colorectal cancer (mCRC) patients treated with irinotecan, the parent compound of SN-38. UGT1A1*28, UGT1A1*60, UGT1A1*93, UGT1A7*3 AND UGT1A9*22 were genotyped in 250 mCRC patients (Cecchin et al., 2009, J Clin Oncol 27:2457-65). The UGT1A7*3 haplotype was the only biomarker for severe hematologic and gastrointestinal toxicity after first cycle treatment and was associated with glucuronidation of SN-38, while UGT1A1*28 was the only biomarker associated with time to progression (Cecchin et al., 2009, J Clin Oncol 27:2457-65). Other studies have concluded that UGT1A1*6 and UGT1A1*28 were significantly associated with toxicity induced by irinotecan (Yang et al., 2018, Asia Pac J Clin Oncol, 14:e479-89). However, results with these biomarkers have been inconsistent (Yang et al., 2018, Asia Pac J Clin Oncol, 14:e479-89). UGT1A encodes a UDP glucuronosyltransferase, which inactivates SN-38 by glucuronidation. Because the SN-38 conjugated to sacituzumab govitecan is protected from glucuronidation (Sharkey et al., 2015, Clin Cancer Res 21:5131-8), the UGT1A1 biomarkers may not be relevant to toxicity of these ADCs. A study by Ocean et al. (2017, Cancer 123:3843-54) of sacituzumab govitecan (SG) in treatment of diverse epithelial cancers found only a slight apparent correlation between UGT1A1 genotype (specifically UGT1A1*28/UGT1A1*28) and toxicity of SG. The UGT1A1*28/UGT1A1*28 was not indicative of dose-limiting toxicity of sacituzumab govitecan in this study.

P38 is a downstream effector kinase of the DNA damage sensor system, starting with activation of ATM, ATR and DNA-PK (Paillas et al., 2011, Cancer Res 71:1041-9). Elevated levels of activated (phosphorylated) MAPK p38 are associated with resistance to SN-38 and treatment of SN-38 resistant cells with the p38 inhibitor SB202190 enhances the cytotoxic effect of SN-38 (Paillas et al., 2011, Cancer Res 71:1041-9). Primary colon cancers of patients sensitive to irinotecan showed decreased levels of phosphorylated p38 (Paillas et al., 2011, Cancer Res 71:1041-9). Levels of phosphorylated p38 may be a biomarker of use for anti-Trop-2 ADCs, with low levels of phosphorylated p38 indicative of sensitivity to ADC, and high levels indicative of resistance (Paillas et al., 2011, Cancer Res 71:1041-9). Further, inhibitors of p38 may be of use in combination therapy with topoisomerase I-inhibiting ADCs in resistant tumors.

Other DDR genes reported to be associated with topoisomerase I inhibitor sensitivity or resistance include PARP, TDP1, XPF, APTX, MSH2, MLH1 and ERCC1 (Gilbert et al., 2012, Br J Cancer 106:18-24). The same biomarkers may be of use to predict sensitivity or resistance to topoisomerase I-inhibiting ADCs. In addition, inhibitory agents against the respective expressed proteins may be of use in combination therapy with topoisomerase I-inhibiting ADCs.

Hoskins et al. (2008, Clin Cancer Res 14:1788-96) examined the effect of genetic variants in CDC45L, NFKB1, PARP1, TDP1, XRCC1 and TOP1 on irinotecan cytotoxicity. SNP markers were identified based on haplotype compositions of subjects of different ethnicities. Haplotype-tagging SNPs (htSNPs) were used to genotype irinotecan-treated patients with advanced colorectal cancer (Hoskins et al., 2008, Clin Cancer Res 14:1788-96). htSNPs in the TOP1 gene were associated with grade 3/4 neutropenia and in the TDP1 gene were associated with response to irinotecan (Hoskins et al., 2008, Clin Cancer Res 14:1788-96). The TOP1 htSNP was located at IVS4+61. The TDP1 SNP was located at IVS12+79 (Hoskins et al., 2008, Clin Cancer Res 14:1788-96). At TOP1 IVS4+61, the G/G genotype showed an 8% incidence of grade 3/4 neutropenia while the A/A genotype showed a 50% incidence (in a small sample size). At TDP1 IVS12+79, the G/G genotype showed a 64% response to irinotecan, while the T/T genotype showed a 25% response (Hoskins et al., 2008, Clin Cancer Res 14:1788-96). A non-significant association was observed between genotype at XRCC1c.1196G>A and clinical response.

Recently, expression of the Schlafen 11 (SLFN11) gene has been identified as a biomarker for sensitivity to DNA damage repair inhibitors, including topoisomerase I inhibitors (Thomas & Pommier, Jun. 21, 2019, Clin Cancer Res [Epub ahead of print]; Ballestrero et al., 2017, J Transl Med 15:199). SLFN11 is a putative DNA/RNA helicase associated with resistance to topoisomerase I and II inhibitors, platinum compounds and other DNA damaging agents, as well as antiviral response (Ballestrero et al., 2017, J Transl Med 15:199). SLFN11 hypermethylation (resulting in decreased expression) is associated with poor prognosis in ovarian cancer and resistance to platinum compounds in lung cancer, while high expression of SLFN11 was correlated with improved survival following chemotherapy in breast cancer (Ballestrero et al., 2017, J Transl Med 15:199). Thus, SLFN11 expression levels and/or methylation status in cancer cells may be predictive of sensitivity to topoisomerase-inhibiting ADCs, alone or in combination with one or more DDR inhibitors.

A novel phosphorylation site at serine residue 506 in the topoisomerase I sequence has been identified as widely expressed in cancer but not in normal tissue and associated with increased sensitivity to camptothecin type topoisomerase I inhibitors (Zhao & Gjerset, 2015, PLoS One 10:e0134929).

Increased expression of c-Met was associated with poor clinical outcome and resistance to inhibitors of topoisomerase II in breast cancer (Jia et al., 2018, Med Sci Monit 24:8239-49). Increased expression of APTX was also reported to be associated with resistance to camptothecin (Gilbert et al., 2012, Br J Cancer 106:18-24).

These and other biomarkers may be predictive of toxicity and/or efficacy of topoisomerase I-inhibiting ADCs.

Biomarkers for Sensitivity to PARP Inhibitors

It is well known in the art that BRCA1/2 mutations are indicative of susceptibility to PARP inhibitors, and in fact the FDA-approved clinical use of PARP inhibitors such as olaparib in ovarian cancer is directed to treatment of patients with germline BRCA mutations. Diagnostic and predictive use of BRCA mutations is not limited to ovarian cancer, but may also apply to other cancer types such as TNBC (see, e.g., Cardillo et al., 2017, Clin Cancer Res 23:3405-15). Similar mutations have been suggested to be indicative of “BRCAness,” such as mutations in the CHEK2, NBN, PTEN and ATM genes (Cardillo et al., 2017, Clin Cancer Res 23:3405-15; Turner et al. 2004, Nat Rev Cancer 4:814-19; Lips et al., 2011, Ann Oncol 22:870-76). Mutations in other genes predisposing to PARP1 sensitivity include PARB2, BRIP1, BARD1, CDK12, RAD51 and p53 (Bitler et al., 2017, Gynecol Oncol 147:695-704; Lui et al., J Clin Pathol 71:957-62; Weber & Ryan, 2015, Pharmacol Ther 149:124-38). BRCA methylation resulting in epigenetic silencing has also been suggested to predispose to PARP inhibitor sensitivity (see, e.g., Bitler et al., 2017, Gynecol Oncol 147:695-704). BRCA 1/2 mutation and silencing occur in about 30% of high grade serous ovarian cancers and frequently results in diminished HR pathway activity (Bitler et al., 2017, Gynecol Oncol 147:695-704). Other biomarkers for PARPi resistance include overexpression of FANCD2, loss of PARPI, loss of CHD4, inactivation of SLFN11 or loss of 53BP1, REV7/MAD2L2, PAXIPI/PTIP or Artemis (Cruz et al., 2018, Ann Oncol 29:1203-10). In addition, secondary mutations may restore function of BRCA1/2 to overcome inhibition of PARP (Cruz et al., 2018, Ann Oncol 29:1203-10).

The effect of changes in RAD51 function on PARP resistance has been examined in BRCA-mutated breast cancer (Cruz et al., 2018, Ann Oncol 29:1203-10). RAD51 is frequently overexpressed in cancers (see, e.g., Wikipedia under “Rad51”). As a key protein in the HR pathway, overexpression of RAD51 in gBRCA1/2 mutants may partially compensate for loss of HR function and decrease susceptibility to PARPi (Cruz et al., 2018, Ann Oncol 29:1203-10). Cruz et al. used exome sequencing and immunostaining of DDR proteins to investigate the mechanism of PARPi resistance in BRCA mutant breast cancer. RAD51 nuclear foci, a surrogate marker for HR functionality, was the only common feature observed in PARPi resistant tumors, while low RAD51 expression was associated with increased response to PARPi (Cruz et al., 2018, Ann Oncol 29:1203-10). These results suggest that use of PARP inhibitors (PARPi) may be contraindicated by the presence of RAD51 foci, while low expression of RAD51 may be a positive biomarker for susceptibility to PARPi. Further, RAD51 inhibitors may be of use in combination with PARP inhibitors. No correlation was observed between RAD51 foci and sensitivity to platinum-based chemotherapeutic agents (Cruz et al., 2018, Ann Oncol 29:1203-10).

The discussion above relates to biomarkers for sensitivity to PARP inhibitors, such as olaparib. They may therefore be relevant to combination therapy using an anti-Trop-2 ADC and a PARP inhibitor. Further, since the biomarkers are indicative of the status of DDR pathways, which may in turn relate to sensitivity to DNA damaging agents like topoisomerase I inhibitors and corresponding ADCs, any such biomarkers may be of use to predict sensitivity to ADCs bearing topo I inhibitors, like SN-38 or DxD.

Other Biomarkers for Sensitivity to Anti-Cancer Agents

It has been suggested that p53 mutations, which are common in cancer, may predispose cancer cells to inhibitors targeted to ATM and/or ATR kinases (Weber & Ryan, 2015, Pharmacol Ther 149:124-38), as well as to combination therapy with ATM and PARP inhibitors (Brandsma et al., 2017, Expert Opin Investig Drugs 26:1341-55).

Sensitivity to the ATR inhibitor AZD6738 was enhanced in ATM deficient xenografts, compared to ATM-proficient tumors, suggesting that synthetic lethality may be achieved by mutations or inhibitors that block both ATM and ATR pathways (Weber & Ryan, 2015, Pharmacol Ther 149:124-38). NSCLC tumors that were deficient in both ATM and p53 showed particular sensitivity to ATR inhibition (Weber & Ryan, 2015, Pharmacol Ther 149:124-38). Synthetic lethality has been observed between the ATM or ATR pathways and multiple components of DDR, including the Fanconi anemia pathway, APE1 inhibitors, functional loss of XRCC1, ERCC1, ERCC4 (XPF) or MRE11A (Weber & Ryan, 2015, Pharmacol Ther 149:124-38; Brandsma et al., 2017, Expert Opin Investig Drugs 26:1341-55). Other defects that increase sensitivity to ATM and/or ATR inhibitors include FANCD2, RAD50, BRCA1 and ATM. These results relate to combination therapies with DNA-damaging ADCs and ATM and/or ATR inhibitors. Where both ATM and ATR regulated pathways are active, use of anti-Trop-2 ADC in combination with both an ATM and an ATR inhibitor may be indicated. Where there is a mutation in an ATM regulated DNA repair pathway, combination therapy with ADC and an ATR inhibitor may be indicated. Similarly, mutations in an ATR regulated pathway may indicate use of ADC in combination with an ATM inhibitor. The person of ordinary skill is aware that ATM and ATR catalyze the initial steps in pathways contain multiple downstream effectors discussed in detail above, and that use of an ATM or ATR inhibitor may be substituted by an inhibitor of a downstream effector in the same DDR pathway.

Synthetic lethality for ATR, based on RNAi experiments, have been suggested for silencing of ATRIP, RAD17, RAD9A, RAD1, HUS1, POLD1, ARID1A and TOPBP1, and these also sensitized cells to VE821 (Brandsma et al., 2017, Expert Opin Investig Drugs 26:1341-55). Loss of CDC25A function is suggested to be associated with ATR inhibitor resistance (Brandsma et al., 2017, Expert Opin Investig Drugs 26:1341-55).

Biomarkers for DNA-PK inhibitor sensitivity include defects in AKT1, CDK4, CDK9, CHK1, IGFR1, mTOR, VHL, RRM2, MYC, MSH3, BRCA1, BRCA2, ATM and other HR associated genes (Brandsma et al., 2017, Expert Opin Investig Drugs 26:1341-55).

Mutations in p53 have been suggested as indicating increased susceptibility to WEE1 inhibitors or to combination therapy with CHK1 inhibitors and DNA damaging agents (Ronco et al., 2017, Med Chem Commun 8:295-319). WEE1 inhibitors are also more effective in cells with lower expression of PKMYT1 and mutations in FANCC, FANCG and BRCA2 (Brandsma et al., 2017, Expert Opin Investig Drugs 26:1341-55).

Nadaraja et al. (Sep. 3, 2019, Acta Oncol, [Epub ahead of print]) examined alterations in transcriptomic profiles of patients with high-grade serous carcinoma (HGSC) receiving first-line platinum-based therapy. A gene expression array was used to detect changes in mRNA, while the protein expression of selected biomarkers was examined by IHC (Nadaraja et al., Sep. 3, 2019, Acta Oncol [Epub ahead of print]). Expression of ARAP1 (ankyrin repeat and PH domain 1) was significantly lower in early progressors vs. late progressors. ARAP1 expression identified 64.7% of early progressors, with a sensitivity of 78.6% (Nadaraja et al., Sep. 3, 2019, Acta Oncol [Epub ahead of print]). These results indicate that ARAP1 expression is indicative of sensitivity to platinum-based anti-cancer agents and may be of use to predict sensitivity to other DNA-damaging agents, such as topoisomerase I-inhibiting ADCs.

A similar study was performed by Ilelis et al. (2018, Pathol Res Pract 214:187-94), using ICH to examine expression of GRIM-19, NF-κB and IKK2 in HGSC patients treated with platinum-based chemotherapy. It was concluded that high IKK2 and NF-κB expression were associated with poor survival and resistance to platinum-based agents, while high expression of GRIM-19 was predictive of higher disease-free survival and negatively associated with relapse. Expression of GRIM-19 may be a useful biomarker for sensitivity to platinum-based therapy and potentially other DNA-damaging treatments, such as topoisomerase I-inhibiting ADCs.

Miao et al. (2019, Cell Mol biol 65:64-72) used quantitative PCR to determine cfDNA levels in breast cancer patients, compared to benign and normal samples. Plasma CEA, CA125 and CA15-3 were also determined. The cfDNA concentration and integrity in breast cancer patients were significantly higher than control groups, and both biomarkers significantly decreased following chemotherapy (Miao et al., 2019, Cell Mol biol 65:64-72). The sensitivity and specificity of cfDNA analysis were significantly higher than those of traditional tumor biomarkers (Miao et al., 2019, Cell Mol biol 65:64-72). Thus, in addition to examining specific biomarkers in cfDNA, the levels of total cfDNA in serum may serve as a biomarker for the presence of cancer and for the efficacy of anti-cancer therapies.

Faltas et al. (2016 Nat Genet 48:1490-99) reported that mutations in L1CAM (L1-cell adhesion molecule) were associated with resistance to chemotherapy (e.g., cisplatin resistance) in metastatic urothelial cancer. The majority of these were missense mutations. The analysis was performed using whole exome sequencing, analyzing 21,522 genes including 250 targeted cancer genes.

These and other known biomarkers may be used to predict sensitivity, resistance or toxicity of ADCs used for cancer treatment alone or in combination with other ant-cancer agents. The person of ordinary skill will be aware that such cancer biomarkers may have other uses, such as increasing diagnostic accuracy, individualizing patient therapy (precision medicine), establishing a prognosis, predicting treatment outcomes and relapse, monitoring disease progression and/or identifying early relapse from cancer therapy.

Kits

Various embodiments may concern kits containing components suitable for treating diseased tissue in a patient. Exemplary kits may contain at least one antibody or ADC as described herein. A kit may also include a drug such as a DDR inhibitor or other known anti-cancer therapeutic agent. If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained in a sterile manner within the containers. Another component that can be included is instructions to a person using a kit for its use

### Additional Exemplary Embodiments

In one aspect provided herein is a method of treating a Trop-2 expressing cancer comprising a) assaying a sample from a human subject with a Trop-2 expressing cancer for the presence of one or more cancer biomarkers; b) detecting one or more biomarkers associated with sensitivity to an anti-Trop-2 antibody-drug conjugate (ADC); and c) treating the subject with an anti-Trop-2 ADC comprising an anti-Trop-2 antibody conjugated to a topoisomerase I inhibitor. In some embodiments the method further comprises d) detecting one or more biomarkers associated with sensitivity to combination therapy with an anti-Trop-2 ADC and a DDR inhibitor; and e) treating the subject with the combination of an anti-Trop-2 ADC and a DDR (DNA damage repair) inhibitor.

In another aspect provided herein is a method of selecting patients to be treated with an anti-Trop-2 antibody-drug conjugate (ADC) comprising a) analyzing a sample from a human cancer patient for the presence of one or more cancer biomarkers; b) detecting one or more biomarkers associated with sensitivity to or toxicity of an anti-Trop-2 ADC; c) selecting patients to be treated with an anti-Trop-2 ADC based on the presence of the one or more biomarkers; and d) treating the selected patients with an anti-Trop-2 ADC. In some embodiments the method further comprises e) selecting patients to be treated with a combination therapy, based on the presence of the one or more biomarkers; and f) treating the patients with a combination of an anti-Trop-2 ADC and a DDR inhibitor.

In some embodiments the anti-Trop-2 ADC is administered to the patient as a neoadjuvant therapy, prior to administration of the at least one other anti-cancer therapy.

In some embodiments the method further comprises e) monitoring the patient for the presence of one or more biomarkers; and f) determining the response of the cancer to the treatment.

In some embodiments the method further comprises monitoring for residual disease or relapse of the patient based on biomarker analysis.

In some embodiments the method further comprises determining a prognosis for disease outcome or progression based on biomarker analysis.

In some embodiments the method further comprises selecting an optimized individual therapy for the patient based on biomarker analysis.

In some embodiments the method further comprises staging the cancer based on biomarker analysis.

In some embodiments the method further comprises stratifying a population of patients for initial therapy based on the biomarker analysis.

In some embodiments the method further comprises recommending supportive therapy to ameliorate side effects of ADC treatment, based on biomarker analysis.

In some embodiments the sample is a biopsy sample from a solid tumor.

In some embodiments the sample is a liquid biopsy sample.

In some embodiments the sample comprises cfDNA, ctDNA or circulating tumor cells (CTCs).

In some embodiments the sample comprises CTCs and the CTCs are analyzed for the presence of one or more cancer biomarkers.

In some embodiments the biomarker is a genetic marker in a DNA damage repair (DDR) gene or an apoptosis gene.

In some embodiments the gene is selected from the group consisting of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NDRG1, WEE1, PPP1R15A, MYBBP1A, SIRT1, ABL1, HRAS, ZNF385B, POLR2K and DDB2.

In some embodiments the biomarkers comprise or consist of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NDRG1, WEE1, PPP1R15A, MYBBP1A, SIRT1, ABL1, HRAS, ZNF385B, POLR2K and DDB2.

In some embodiments the biomarkers comprise or consist of AEN, MSH2, MYBBP1A, SART1, SIRT1, USP28, CDKN1A, ABL1, TP53, BAG6, BRCA1, BRCA2, BRSK2, CHEK2, ERN1, FHIT, HIPK2, HRAS, LGALS12, MSH6, ZNF385B and ZNF622.

In some embodiments the biomarkers comprise or consist of BRCA1, BRCA2, CHEK2, MSH2, MSH6, TP53, CDKN1A, BAG6, BRSK2, ERN1, FHIT, HIPK2, LGALS12, ZNF622, AEN, SART1 and USP28.

In some embodiments the biomarkers comprise or consist of POLR2K, DDB2, GADD45B, WEE1, TGFB1, NDRG1 and PPP1R15A.

In some embodiments the biomarkers comprise or consist of GADD45B, TGFB1, NRG1, WEE1 and PPP1R15A.

In some embodiments the biomarkers comprise or consist of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NRG1, WEE1 and PPP1R15A.

In some embodiments the gene is selected from the group consisting of BRCA1, BRCA2, PTEN, ERCC1 and ATM.

In some embodiments the biomarkers comprise or consist of BRCA1, BRCA2, PTEN, ERCC1 and ATM.

In some embodiments the biomarker is a single nucleotide polymorphism that results in a substitution mutation selected from the group consisting of E155K in ABL1, G706S in ABL1, V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, G12V in HRAS, A278V in LGALS12, N127S in MSH2, S625F in MSH6, H680Y in MYBBP1A, R373Q in SART1, E113Q in SIRT1, *394S in TP53, R282G in TP53, T377P in in TP53, E271K in TP53, Y220C in TP53, E180* in TP53, I987L in USP28, R370Q in ZNF385B and A437E in ZNF622.

In some embodiments the biomarkers are a plurality of single nucleotide polymorphisms that result in a substitution comprising or consisting of E155K in ABL1, G706S in ABL1, V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, G12V in HRAS, A278V in LGALS12, N127S in MSH2, S625F in MSH6, H680Y in MYBBP1A, R373Q in SART1, E113Q in SIRT1, *394S in TP53, R282G in TP53, T377P in in TP53, E271K in TP53, Y220C in TP53, E180* in TP53, I987L in USP28, R370Q in ZNF385B and A437E in ZNF622.

In some embodiments the biomarkers are a plurality of single nucleotide polymorphisms that result in a substitution comprising or consisting of V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, N127S in MSH2, S625F in MSH6, R373Q in SART1, *394S in TP53, R282G in TP53, T377P in in TP53, E271K in TP53, Y220C in TP53, E180* in TP53, and I987L in USP28.

In some embodiments the biomarker is a frameshift mutation selected from the group consisting of K1110fs in BAG6, R32fs in CDKN1A, DC33fs in CDKN1A and EG60fs in CDKN1A.

In some embodiments the biomarkers are a plurality of frameshift mutations comprising or consisting of K1110fs in BAG6, R32fs in CDKN1A, DC33fs in CDKN1A, and EG60fs in CDKN1A.

In some embodiments the biomarker is an increase or decrease in gene expression in the cancer compared to corresponding normal tissue for a gene selected from the group consisting of POLR2K, DDB2, GADD45B, WEE1, TGFB1, NDRG1 and PPP1R15A.

In some embodiments the biomarkers are a plurality of increases or decreases in gene expression in the cancer compared to corresponding normal tissue comprising or consisting of POLR2K, DDB2, GADD45B, WEE1, TGFB1, NDRG1 and PPP1R15A.

In some embodiments the gene is selected from the group consisting of BRCA1, BRCA2, PTEN, ERCC1 and ATM.

In some embodiments the biomarkers comprise or consist of BRCA1, BRCA2, PTEN, ERCC1 and ATM.

In some embodiments the biomarker is selected from the group consisting of a mutation, insertion, deletion, chromosomal rearrangement, SNP (single nucleotide polymorphism), DNA methylation, gene amplification, RNA splice variant, miRNA, increased expression of a gene, decreased expression of a gene, phosphorylation of a protein and dephosphorylation of a protein.

In some embodiments the sample assay comprises next generation sequencing of DNA or RNA.

In some embodiments the topoisomerase I inhibitor is SN-38 or DxD.

In some embodiments the anti-Trop-2 ADC is selected from the group consisting of sacituzumab govitecan and DS-1062.

In some embodiments the DDR inhibitor is an inhibitor of 53BP1, APE1, Artemis, ATM, ATR, ATRIP, BAP1, BARD1, BLM, BRCA1, BRCA2, BRIP1, CDC2, CDC25A, CDC25C, CDK1, CDK12, CHK1, CHK2, CSA, CSB, CtIP, Cyclin B, Dna2, DNA-PK, EEPD1, EME1, ERCC1, ERCC2, ERCC3, ERCC4, Exo1, FAAP24, FANC1, FANCM, FAND2, HR23B, HUS1, KU70, KU80, Lig III, Ligase IV, Mdm2, MLH1, MRE11, MSH2, MSH3, MSH6, MUS81, MutSα, MutSβ, NBS1, NER, p21, p53, PALB2, PARP, PMS2, Pol μ, Pol β, Pol δ, Pol ε, Pol κ, Pol λ, PTEN, RAD1, RAD17, RAD23B, RAD50, RAD51, RAD51C, RAD52, RAD54, RAD9, RFC2, RFC3, RFC4, RFC5, RIF1, RPA, SLX1, SLX4, TopBP1, USP11, WEE1, WRN, XAB2, XLF, XPA, XPC, XPD, XPF, XPG, XRCC1, or XRCC4.

In some embodiments the DDR inhibitor is an inhibitor of PARP, CDK12, ATR, ATM, CHK1, CHK2, CDK12, RAD51, RAD52 or WEE1.

In some embodiments the PARP inhibitor is selected from the group consisting of olaparib, talazoparib (BMN-673), rucaparib, veliparib, niraparib, CEP 9722, MK 4827, BGB-290 (pamiparib), ABT-888, AG014699, BSI-201, CEP-8983, E7016 and 3-aminobenzamide.

In some embodiments the CDK12 inhibitor is selected from the group consisting of dinaciclib, flavopiridol, roscovitine, THZ1 and THZ531.

In some embodiments the RAD51 inhibitor is selected from the group consisting of B02 ((E)-3-benzyl-2(2-(pyridin-3-yl)vinyl) quinazolin-4(3H)-one); RI-1 (3-chloro-1-(3,4-dichlorophenyl)-4-(4-morpholinyl)-1H-pyrrole-2,5-dione); DIDS (4,4′-diisothiocyanostilbene-2,2′-disulfonic acid); halenaquinone; CYT-0851, IBR₂ and imatinib.

In some embodiments the ATM inhibitor is selected from the group consisting of Wortmannin, CP-466722, KU-55933, KU-60019, KU-59403, AZD0156, AZD1390, CGK733, NVP-BEZ 235, Torin-2, fluoroquinoline 2 and SJ573017.

In some embodiments the ATR inhibitor is selected from the group consisting of Schisandrin B, NU6027, BEZ235, ETP46464, Torin 2, VE-821, VE-822, AZ20, AZD6738 (ceralasertib), M4344, BAY1895344, BAY-937, AZD6738, BEZ235 (dactolisib), CGK 733 and VX-970.

In some embodiments the CHK1 inhibitor is selected from the group consisting of XL9844, UCN-01, CHIR-124, AZD7762, AZD1775, XL844, LY2603618, LY2606368 (prexasertib), GDC-0425, PD-321852, PF-477736, CBP501, CCT-244747, CEP-3891, SAR-020106, Arry-575, SRA737, V158411 and SCH 900776 (MK-8776).

In some embodiments the CHK2 inhibitor is selected from the group consisting of NSC205171, PV1019, CI2, CI3, 2-arylbenzimidazole, NSC109555, VRX0466617 and CCT241533.

In some embodiments the WEE1 inhibitor is selected from the group consisting of AZD1775 (MK1775), PD0166285 and PD407824.

In some embodiments the DDR inhibitor is selected from the group consisting of mirin, M1216, NSC19630, NSC130813, LY294002 and NU7026.

In some embodiments the DDR inhibitor is not an inhibitor of PARP or RAD51.

In some embodiments the anti-Trop-2 ADC comprises an hRS7 antibody comprising the light chain CDR sequences CDR1 (KASQDVSIAVA, SEQ ID NO:1); CDR2 (SASYRYT, SEQ ID NO:2); and CDR3 (QQHYITPLT, SEQ ID NO:3) and the heavy chain CDR sequences CDR1 (NYGMN, SEQ ID NO:4); CDR2 (WINTYTGEPTYTDDFKG, SEQ ID NO:5) and CDR3 (GGFGSSYWYFDV, SEQ ID NO:6).

In some embodiments the method further comprises treating the subject with an anti-cancer agent selected from the group consisting of olaparib, rucaparib, talazoparib, veliparib, niraparib, acalabrutinib, temozolomide, atezolizumab, pembrolizumab, nivolumab, ipilimumab, pidilizumab, durvalumab, BMS-936559, BMN-673, tremelimumab, idelalisib, imatinib, ibrutinib, eribulin mesylate, abemaciclib, palbociclib, ribociclib, trilaciclib, berzosertib, ipatasertib, uprosertib, afuresertib, triciribine, ceralasertib, dinaciclib, flavopiridol, roscovitine, G1T38, SHR6390, copanlisib, temsirolimus, everolimus, KU 60019, KU 55933, KU 59403, AZ20, AZD0156, AZD1390, AZD1775, AZD2281, AZD5363, AZD6738, AZD7762, AZD8055, AZD9150, BAY-937, BAY1895344, BEZ235, CCT241533, CCT244747, CGK 733, CID44640177, CID1434724, CID46245505, CHIR-124, EPT46464, FTC, VE-821, VRX0466617, VX-970, LY294002, LY2603618, M1216, M3814, M4344, M6620, MK-2206, NSC19630, NSC109555, NSC130813, NSC205171, NU6027, NU7026, prexasertib (LY2606368), PD0166285, PD407824, PV1019, SCH900776, SRA737, BMN 673, CYT-0851, mirin, Torin-2, fluoroquinoline 2, fumitremorgin C, curcurmin, Ko143, GF120918, YHO-13351, YHO-13177, XL9844, Wortmannin, lapatinib, sorafenib, sunitinib, nilotinib, gemcitabine, bortezomib, trichostatin A, paclitaxel, cytarabine, cisplatin, oxaliplatin and carboplatin.

In some embodiments the cancer is selected from the group consisting of breast cancer, triple negative breast cancer (TNBC), HR+/HER2− metastatic breast cancer, urothelial cancer, small cell lung cancer (SCLC), non-small cell lung cancer (NSCLC), colorectal cancer, stomach cancer, bladder cancer, renal cancer, ovarian cancer, uterine cancer, endometrial cancer, cervical cancer, prostate cancer, esophageal cancer, pancreatic cancer, brain cancer, liver cancer and head-and-neck cancer. In some embodiments the cancer is urothelial cancer. In some embodiments the cancer is metastatic urothelial cancer. In some embodiments the cancer is treatment resistant urothelial cancer. In some embodiments the cancer is resistant to treatment with platinum-based and checkpoint inhibitor (CPI) (e.g., anti-PD1 antibody or anti-PD-L1 antibody) based therapy. In some embodiments the cancer is metastatic TNBC.

In another aspect provided herein is a method of predicting clinical outcome in a subject with a Trop-2 expressing cancer following treating with an anti-Trop-2 ADC, comprising assaying a sample from a human subject with a Trop-2 expressing cancer for the presence of one or more cancer biomarkers, wherein the presence or absence of one or more cancer biomarkers is predictive of clinical outcome in the subject.

In some embodiments the presence or absence of one or more cancer biomarkers is predictive of the efficacy of treatment with an anti-Trop-2 ADC, wherein the ADC comprises an inhibitor of topoisomerase I.

In some embodiments the presence or absence of one or more cancer biomarkers is predictive of the efficacy or safety of treatment with a combination of anti-Trop-2 ADC and a DDR inhibitor.

In some embodiments the presence or absence of one or more cancer biomarkers is predictive of the efficacy or safety of treatment with a combination of anti-Trop-2 ADC and a standard anti-cancer therapy.

In some embodiments the method further comprises predicting recurrence-free interval, overall survival, disease-free survival or distant recurrence-free interval following treatment with an anti-Trop-2 ADC.

### EXAMPLES

Various embodiments of the present invention are illustrated by the following examples, without limiting the scope thereof.

### Example 1. Sacituzumab Govitecan in Treatment-Resistant Metastatic Urothelial Cancer (mUC) and Biomarkers for Sensitivity

### Summary

Patients with metastatic urothelial cancer (mUC) who progress after platinum-based and checkpoint inhibitor (CPI) therapy have limited options. Sacituzumab govitecan is an antibody-drug conjugate (ADC) comprising a humanized monoclonal anti-Trop-2 antibody conjugated to the cytotoxic agent SN-38. A phase Eli single-arm, multicenter trial (NCT01631552) evaluated the safety and activity of sacituzumab govitecan in pretreated mUC with progression after ≥1 prior systemic therapy.

Patients received intravenous sacituzumab govitecan (10 mg/kg) on day 1 and 8 of 21-day cycles until progression or unacceptable toxicity. Endpoints included safety, investigator-evaluated objective response rate (ORR per RECIST 1.1), clinical benefit rate, duration of response (DOR), progression-free survival (PFS), and overall survival (OS). Sequencing analysis of differentially mutated and expressed genes and pathways was performed in subsets of tumors from responders and non-responders.

Forty-five patients treated at the recommended phase 2 dose were enrolled (median age, 67 [range: 49-90] years; 91% male; median 2 [range: 1-6] prior therapies; 69% with ECOG PS score 1; 73% with visceral metastases [33% with liver metastases]) received ≥1 sacituzumab govitecan dose. ORR was 31% (14/45; 2 complete, 12 partial responses). Median DOR was 12.9 months, PFS 7.3 months, and OS 16.3 months. ORR was 33% (5/15) for patients with liver metastases, 24% (4/17) for prior CPI-treated patients (median 3 prior therapy lines), and 27% (4/15) for prior CPI- and platinum-treated patients. Most frequent grade ≥3 adverse events were neutropenia (38%), anemia (13%), hypophosphatemia (11%), diarrhea (9%), fatigue (9%), and febrile neutropenia (7%). Sequencing of tumors from responders showed enrichment in DNA repair and apoptosis pathway molecular alterations.

Based on the results of the study reported herein, we conclude that sacituzumab govitecan demonstrated significant clinical activity in resistant mUC, with manageable toxicity.

Introduction

Patients with metastatic urothelial cancer (mUC) who progress after platinum-based chemotherapy and immune checkpoint inhibitor (CPI) therapy have poor outcomes and limited treatment options (Di Lorenzo et al., 2015, Medicine (Baltimore) 94:e2297; Vlachostergios et al., 2018, Bladder Cancer 4:247-59). The therapeutic landscape for mUC was recently expanded by the approval of several CPIs (checkpoint inhibitors) for chemotherapy-resistant mUC. However, only approximately 15%-21% of patients respond to these agents (Vlachostergios et al., 2018, Bladder Cancer 4:247-59; Bellmunt et al., 2017, N Eng J Med 37:1015-26; Patel et al., 2018, Lancet Oncol 19:51-64; Powles et al., 2017, JAMA Oncol 3:e172411; Rosenberg et al., 2016, Lancet 387:1909-20). Patients with disease progression on CPIs currently have no approved therapeutic options (Di Lorenzo et al., 2015, Medicine (Baltimore) 94:e2297; Bellmunt et al., 2017, N Eng J Med 37:1015-26). Developing effective regimens for these patients remains an urgent unmet need.

Sacituzumab govitecan is a novel antibody-drug conjugate (ADC) targeting the trophoblast cell surface antigen 2 (Trop-2) (Goldenberg et al., 2015, Oncotarget 6:22496-512). Trop-2 is a transmembrane calcium signal transducer highly expressed in most epithelial cancers (Trerotola et al., 2013, Oncogene 32:222-33; Avellini et al., 2017, Oncotarget 8:58642-53; Shvartsur & Bonavida, 2015, Genes Cancer 6:84-105; Stepan et al., 2011, J Histochem Cytochem 59:701-10; Goldenberg et al., 2018, Oncotarget 9:28989-29006). Elevated Trop-2 expression is associated with poor prognosis and plays a key role in cell transformation and proliferation, with higher expression seen in metastatic versus early stage disease (Trerotola et al., 2013, Oncogene 32:222-33; Avellini et al., 2017, Oncotarget 8:58642-53; Shvartsur & Bonavida, 2015, Genes Cancer 6:84-105; Stepan et al., 2011, J Histochem Cytochem 59:701-10; Goldenberg et al., 2018, Oncotarget 9:28989-29006).

Sacituzumab govitecan consists of an anti-Trop-2 humanized monoclonal antibody hRS7 IgG1κ coupled to SN-38, the active metabolite of the topoisomerase 1 inhibitor irinotecan (Goldenberg et al., 2018, Oncotarget 9:28989-29006). This coupling is achieved using a unique hydrolyzable CL2A linker (Goldenberg et al., 2015, Oncotarget 6:22496-512; Goldenberg et al., 2018, Oncotarget 9:28989-29006; Cardillo et al., 2011, Clin Cancer Res 17:3157-69; Cardillo et al., 2015, Bioconjug Chem 26:919-31; Starodub et al., 2015, Clin Cancer Res 21:3870-78). Sacituzumab govitecan is a novel ADC with a much higher drug-antibody ratio than other ADCs (up to 8 molecules of SN-38 per antibody), whereas other ADCs generally have a 2:1 to 4:1 ratio (Goldenberg et al., 2018, Oncotarget 9:28989-29006; Challita et al., 2016, Cancer Res 76:3003-13). After binding to Trop-2, hRS7 (in a free or conjugated form) is internalized, delivering SN-38 inside tumor cells (Cardillo et al., 2011, Clin Cancer Res 17:3157-69). The unique hydrolyzable linker of sacituzumab govitecan also enables SN-38 to be released into the tumor microenvironment such that sacituzumab govitecan-bound tumor cells are killed by intracellular uptake of SN-38 and adjacent tumor cells by SN-38 released extracellularly, where SN-38 readily passes through the cell surface membrane of cells in close proximity (Goldenberg et al., 2018, Oncotarget 9:28989-29006; Cardillo et al., 2015, Bioconjug Chem 26:919-31; Starodub et al., 2015, Clin Cancer Res 21:3870-78).

The safety and efficacy of sacituzumab govitecan was assessed initially in a phase Eli basket design, open-label, single-arm, multicenter trial (IMMU-132-01; NCT01631552) in patients with advanced epithelial cancers who received at least one prior therapy for metastatic disease (Starodub et al., 2015, Clin Cancer Res 21:3870-78; Ocean et al., 2017, Cancer 123:3843-54). Encouraging clinical activity was reported in four cancer types from this study: triple-negative and hormone receptor-positive/HER2-negative breast cancer (Bardia et al., 2017, J Clin Oncol 35:2141-48; Bardia et al., 2019, N Engl J Med 380:741-51; Bardia et al., 2018, J Clin Oncol 36(suppl):1004), pretreated small-cell lung cancer (Gray et al., 2017, Clin Cancer Res 23:5711-19), and non-small-cell lung cancer (Heits et al., 2017, J Clin Oncol 35:2790-97). In addition, Faltas and colleagues reported early results from the phase I portion of the study in patients with mUC (Faltas et al., 2016, Clin Genitourin Cancer 14:e75-9). Herein, we report the safety and efficacy findings for sacituzumab govitecan in pretreated patients with mUC.

Materials and Methods

Patients—Eligible patients 18 years of age or older with histologically confirmed mUC who had relapsed after or were refractory to at least one prior standard therapeutic regimen were enrolled. All patients had metastatic disease measurable by Response Evaluation Criteria in Solid Tumors, version 1.1 (RECIST 1.1) at the time of enrollment. Patients were required to have an Eastern Cooperative Oncology Group (ECOG) performance status of 0 to 1 with expected survival ≥6 months and adequate hepatic, renal, and hematologic function. Patients had to be ≥2 weeks beyond previous line of treatment, including anti-cancer therapy or high-dose systemic corticosteroid, or major surgery, and had to be recovered from all acute toxicities to grade 1 or less (except alopecia). Patients with stable brain metastases could be included only if they were ≥2 weeks beyond high-dose steroid treatment. Pre-selection of patients based on tumor Trop-2 expression was not required.

Study Design—Based on data from the previously reported phase I portion of the study and early safety data from the phase II portion of this study, the 10 mg/kg dose of sacituzumab govitecan was determined to the be maximum tolerated dose (Starodub et al., 2015, Clin Cancer Res 21:3870-8; Ocean et al., 2017, Cancer 123:3843-54). Sacituzumab govitecan was administered intravenously without the requirement for premedication on days 1 and 8 every 21 days of a 3-week treatment cycle, until unacceptable toxicity or disease progression. Hematopoietic growth factors or blood transfusions were allowed at the investigator's discretion, but not prior to the first dose. Other supportive care (antiemetics, anti-diarrheal medications, or bone-stabilizing agents) were allowed as medically needed.

The primary objectives of the phase I and II portions of the study were to define a maximum tolerated dose and to evaluate the safety and efficacy of sacituzumab govitecan, respectively. Additional secondary objectives included assessment of pharmacokinetics and immunogenicity, which were previously reported by Ocean and colleagues (Ocean et al., 2017, Cancer 123:3843-54). Safety evaluations included adverse events (AEs), serious adverse events (SAEs), laboratory safety evaluations, vital signs, physical examinations, and 12-lead electrocardiograms (ECG; performed at baseline, after completion of the infusion on day 1 of every even-numbered treatment cycle, at the end of treatment, and at the end of the study). AEs were graded according to the National Cancer Institute Common Terminology Criteria for Adverse Events, version 4.0.

Staging CT/magnetic resonance imaging (MRI) scans were obtained at baseline and at 8-week intervals from the start of treatment until progression requiring treatment discontinuation. Confirmatory CT/MRI scans were obtained no sooner than 4 weeks after an initial partial response (PR) or complete response (CR). Subsequent scans were done at 8-week intervals after the confirmatory scan. Patients with evidence of clinical benefit were permitted to receive treatment following disease progression. Response was assessed by investigators using RECIST, version 1.1. Efficacy endpoints included objective response rate (ORR), time to response, duration of response (DOR), clinical benefit rate (CBR; defined as CR, PR, or stable disease ≥6 months), progression-free survival (PFS), and overall survival (OS).

Biomarker Analysis—To obtain insights into the underlying biology of response to sacituzumab govitecan, we performed whole-exome sequencing (WES) and RNA sequencing (RNAseq) of available tumors from responders and non-responders under a separate Institutional Review Board-approved protocol with written informed consent. Differentially mutated and expressed genes and pathways were analyzed between responders and non-responders, focusing on molecular alterations in pathways involved in mediating the cytotoxic effects of SN-38, the active moiety of sacituzumab govitecan. To determine the cellular processes that mediate response to sacituzumab govitecan, single-sample gene set enrichment analysis (GSEA) was performed on each tumor.

Fresh frozen and formalin fixed paraffin embedded (FFPE) samples were retrospectively collected from banked excess tissue from archival primary (TURBT, cystectomy) and metastatic (core biopsy) specimens of 14 patients with a diagnosis of urothelial carcinoma at WCM-NYP who were enrolled in the trial. All tumor samples consisted of conventional UC. All pathology specimens were reviewed and reported by board-certified genitourinary pathologists in the Department of Pathology at WCM/NYP.

DNA extraction and whole-exome sequencing—The whole-exome sequencing (WES) protocol used in this study has been previously described (Di Lorenzo et al., 2015, Medicine (Baltimore) 94:e2297; Vlachostergios et al., 2018, Bladder Cancer 4:247-59). After macrodissection of target lesions, tumor DNA was extracted from FFPE or cored OCT-cryopreserved tumors using the Promega MAXWELL® 16 MDx (Promega, Madison, Wis., USA). Germline DNA was extracted from normal tissue adjacent to the tumor, using the same method. Pathological review by one of the WCM/NYP pathologists confirmed the diagnosis and determined tumor content. A minimum of 200 ng of DNA was used for WES. DNA quality was determined by TapeStation Instrument (Agilent Technologies, Santa Clara, Calif.) and was confirmed by real-time PCR before sequencing. Sequencing was performed using Illumina HiSeq 2500 (2×100 bp). A total of 21,522 genes were analyzed with an average coverage of 85× using Agilent HaloPlex Exome (Agilent Technologies, Santa Clara, Calif.).

Whole-exome sequencing data processing pipeline—All of the sample data were processed through the computational analysis pipeline at the Institute for Precision Medicine at Weill Cornell, New York Presbyterian Hospital (IPM-Exome-pipeline) (Vlachostergios et al., 2018, Bladder Cancer 4:247-59). Raw reads quality was assessed with FASTQC and were aligned to the GRCh37 human reference genome (Vlachostergios et al., 2018, Bladder Cancer 4:247-59). Pipeline outputs include segment DNA copy number data, somatic copy-number aberrations (CNAs) and putative somatic single nucleotide variants (SNVs).

Single nucleotide variations—We developed a consensus somatic SNVs calling pipeline to enhance the accuracy of these calls. SNVs were identified in the paired tumor-normal samples using MuTect2, Strelka, VarScan, and SomaticSniper, and only the SNVs identified by at least 2 mutation callers were retained. Indels (insertions or deletions) were identified using Strelka and VarScan and only those identified by both tools were retained. The identified somatic alterations were further filtered using the following criteria: (a) read depth for both tumor and matched normal samples was ≥10 reads, (b) the variant allele frequency (VAF) in tumor samples was ≥5% and greater than 3 reads harboring the mutated allele, (c) the VAF of matched normal was ≤1% or there was just one read with the mutated allele. The variants were annotated using Oncotator (version 1.9); the dbSNPs amongst the mutation calls, unless also found in COSMIC database, were filtered out. For IPMs samples, the promiscuous mutation calls, previously identified internally as artifacts for Haloplex, were also excluded from the final list of mutations. Fischer's exact test was applied to a matrix of gene counts of mutated and wild types phenotypes in responders and non-responders for a given pathway to identify if that pathway was enriched in either of the two patient response groups. Oncoprint was created for the selected mutations using the ‘ComplexHeatmap’ Bioconductor R package.

RNA extraction, RNA sequencing, and data analysis—RNA was extracted from frozen material for RNA-sequencing (RNA-seq) using Promega MAXWELL® 16 MDx instrument, (MAXWELL® 16 LEV simplyRNA Tissue Kit). Specimens were prepared for RNA sequencing using TruSeq RNA Library Preparation Kit v2 or RIBOZERO®. RNA integrity was verified using the Agilent Bioanalyzer 2100 (Agilent Technologies). cDNA was synthesized from total RNA using SUPERSCRIPT® III (Invitrogen). Sequencing was then performed on GAII, HiSeq 2000, or HiSeq 2500. All reads were independently aligned with STAR_2.4.0fl (Bellmunt et al., 2017, N Eng J Med 37:1015-26) for sequence alignment against the human genome sequence build GRCh37, downloaded via the UCSC genome browser, and SAMTOOLS v0.1.19 (Patel et al., 2018, Lancet Oncol 19:51-64) for sorting and indexing reads. The number of reads mapped to each transcript was quantified as counts using the HTSeq-count software. The normalized transcript abundance was quantified as fragments per kb of exon per million fragments mapped (FPKM) using Cufflinks (2.0.2) (Powles et al., 2017, JAMA Oncol 3:e172411), together with GENCODE v23 (Rosenberg et al., 2016, Lancet 387:1909-20) GTF file for annotations. Rstudio (1.0.136) with R (v3.3.2) and ggplot2 (2.2.1) were used for the statistical analysis and the generation of Figures.

RNAseq data quantification, integration, and expression analysis—The mRNA gene expression for 17 UC tumors was quantified as Fragments Per Kilobase of transcript per Million (FPKMs). The FPKM values were log transformed for further analyses. Differential gene expression (DGE) between tumors from responders and non-responders was performed on the counts data using the Bioconductor package DESeq2. The threshold to select for differentially regulated genes was determined at fold change of >2 for upregulated and <−2 for downregulated genes and results were deemed significant at an adjusted p-value of 0.05 (Benjamini-Hochberg correction).

Gene Set Enrichment Analysis—Differential gene expression (DGE) analysis was performed on the RNAseq counts using the Bioconductor R package DESeq. The differentially expressed genes between the responder and non-responder patient groups were identified (upregulated in responders: the log fold change (LFC)>2, downregulated in responders: LFC<−2, adjusted p-value<0.001) and visualized in a heatmap using the ‘pheatmap’ package in R. A pre-ranked Gene Set Enrichment Analysis (GSEA) was applied to the ranked list of all genes, ordered by their LFC values obtained from the DGE analysis. Gene sets available through the Gene Ontology Biological Pathways collection in the Molecular Signatures Database (Goldenberg et al., 2015, Oncotarget 6:22496-512) were used for the GSEA analysis. Two significant pathways from the GSEA analysis, namely HALLMARK_P53_PATHWAY and HALLMARK_APOPTOSIS (FDR<0.10), were further analyzed to obtain individual pathway enrichment scores for each sample using the single sample GSEA (ssGSEA), which was implemented on the RNAseq FPKMs using the ‘gsva’ R package. P-values were obtained from the Mann-Whitney statistical test applied between the responder and non-responder patient groups.

Statistics—Efficacy and safety analyses reported herein include all patients who received at least one dose of sacituzumab govitecan at the 10 mg/kg dose level, regardless of enrollment in the phase I or phase II portion of the study, which comprised 45 patients who were enrolled from September 2014 to June 2017. The data cut-off date for this analysis was Sep. 1, 2018. ORR and CBR were calculated with 95% confidence intervals estimated by the Clopper-Pearson method (Clopper & Pearson, 1934, Biometrika 26:404-13). PFS, OS, and time-to-event endpoints were analyzed by the Kaplan-Meier method, with medians and corresponding 95% confidence intervals determined by the Brookmeyer and Crowley method with log-log transformation. Descriptive statistics were used to characterize AEs. Fischer's exact test was applied to a matrix of pathway-associated gene counts of mutated and wild type phenotypes between responders and non-responders to identify pathways enriched in either of the two response groups. P-values for single sample GSEA (ssGSEA) enrichment score differences between the responder and non-responder patient groups were obtained from the Mann-Whitney statistical test.

Results

Forty-five patients (median age, 67 years; range 49 to 90 years) received at least one dose of sacituzumab govitecan at the 10 mg/kg dose level and were included in the analysis. Seventeen of those patients had received prior CPI treatment and 15 of the patients received prior CPI and platinum-based treatment. Patient demographics and baseline characteristics are shown in Table 1. Patients received a median of 2 prior lines of therapy (range, 1 to 6), including prior platinum-based chemotherapy (93.3%) and prior CPI (37.8%). The majority of patients (33 of 45 [73%]) had visceral involvement, primarily liver (n=15) and lung (n=27) metastases. Forty-four percent of patients had 2 to 3 Bellmunt risk factors (Table 1).

TABLE 1
Patient Demographics and Baseline Characteristics

| Characteristic (Overall mUC Population) | N = 45 | |
|---|---|---|
| Median age, years (range) | 67 | (49-90) |
| Male, n (%) | 41 | (91.1) |
| Race, n (%) | | |
| White | 39 | (86.7) |
| Black | 2 | (4.4) |
| Asian | 1 | (2.2) |
| Other | 2 | (4.4) |
| Not reported | 1 | (2.2) |
| ECOG PS, n (%) | | |
| 0 | 14 | (31.1) |
| 1 | 31 | (68.9) |
| Any visceral disease, n (%) | 33 | (73.3) |
| Visceral metastatic sites,* n (%) | | |
| Lung | 27 | (60.0) |
| Liver | 15 | (33.3) |
| Other Visceral | 5 | (11.1) |
| Median prior anticancer regimens (range) | 2 | (1-6) |
| Line of prior therapy, n (%) | | |
| ≤2 lines | 28 | (62.2) |
| ≥3 lines | 17 | (37.8) |
| Prior therapies,^{†} n (%) | | |
| Prior platinum combinations | 42 | (93.3) |
| Prior immune CPIs | 17 | (37.8) |
| Prior platinum combinations + immune CPIs | 15 | (33.3) |
| Bellmunt risk groups,^{‡} n (%) | | |
| 0 risk factors | 9 | (20.0) |
| 1 risk factor | 16 | (35.6) |
| 2 risk factors | 16 | (35.6) |
| 3 risk factors | 4 | (8.9) |
| Characteristic (CPI-Treated Subgroup) | n = 17 | |
| Age (y), median (range) | 70 | (56-90) |
| ECOG PS, n (%) | | |
| 0 | 1 | (5.9) |
| 1 | 16 | (94.1) |
| Median prior anticancer regimens (range) | 3 | (1-6) |
| Line of prior therapy, n (%) | | |
| ≤2 lines | 5 | (29) |
| ≥3 lines | 12 | (70.6) |

*Categories are not mutually exclusive.
^{†}Bacillus Calmette-Guérin immunotherapy was not considered a prior therapy.
^{‡}Risk factors are ECOG PS > 0, presence of liver metastases, and hemoglobin < 10 g/dL.

The median duration of follow-up was 15.7 months (range, 1 to 39.6 months). Patients received a median of 8 cycles of sacituzumab govitecan (16 doses; range, 1 to 90 doses) with median treatment duration of 5.2 months (range, 0.03 to 32.3 months).

Dose reductions occurred in 40% (18 of 45) of patients (12 of 18 patients had only one dose reduction). Nine patients received treatment for more than 12 months. Thirty-nine (87%) patients discontinued treatment, primarily due to disease progression (Table 2). Five patients continued to receive therapy at the data cut-off date of September 2018 (3 responders, 1 patient with stable disease [SD], and 1 patient who continued therapy after a drug holiday and subsequent progression after a previously documented CR). As of the data cut-off date, 28 deaths have been reported (17 during the follow-up period), with 26 due to disease progression, 1 due to myocardial infarction after end of study, and 1 due to unknown reasons.

TABLE 2
Summary of Reasons for Treatment Discontinuation

| Variable | Patients (N = 45) | |
|---|---|---|
| Permanently discontinued treatment, n (%) | 39 | (86.7) |
| Progressive disease | 29* | (64.4) |
| Treatment-related AE | 5^{†} | (11.1) |
| Consent withdrawn | 2 | (4.4) |
| Investigator decision | 1 | (2.2) |
| Other | 2 | (4.4) |

*2 of 29 patients discontinued due to AEs unrelated to study drug that were related to disease progression.
^{†}Two additional patients discontinued due to AEs unrelated to study drug that were related to disease progression.

Tolerability of Sacituzumab Govitecan—The most common AEs were diarrhea, nausea, fatigue, and neutropenia; grade ≥3 AEs observed in ≥5% of patients also included hypophosphatemia and febrile neutropenia (Table 3). Growth factor support was administered to 24.4% (11 of 45) of patients. No cases of peripheral neuropathy or cardiovascular AEs of grade 3 or higher were reported. Eleven percent (5 of 45) of patients discontinued treatment due to AEs considered likely drug related by the investigator (including grade 3 diarrhea, grade 2 pouchitis, grade 2 pruritus/itching, grade 3 maculopapular rash/pruritus, and grade 3 hypertension). Twenty-one of the 45 patients (46.7%) had one or more SAEs; those occurring in more than one patient included febrile neutropenia, diarrhea, and neutrophil count decreased (2 patients each). No AEs leading to death or treatment-related deaths were reported.

TABLE 3
Adverse Events Observed in ≥ 20% of Patients
Regardless of Causality (N = 45)

| Patients | | | |
|---|---|---|---|
| | Any Grade | Grade 3 | Grade 4 |

| Event | No. of patients with event (%) | | | | |
|---|---|---|---|---|---|
| Any adverse event | 45 | (100) | 25 | (55.6) | 8 (17.8) |
| Diarrhea | 31 | (68.9) | 4 | (8.9) | 0 |
| Nausea | 30 | (66.7) | 1 | (2.2) | 0 |
| Fatigue | 26 | (57.8) | 4 | (8.9) | 0 |
| Neutropenia* | 23 | (51.1) | 10 | (22.2) | 7 (15.6) |
| Constipation | 20 | (44.4) | 0 | | 0 |
| Alopecia | 18 | (40.0) | 0 | | 0 |
| Decreased appetite | 17 | (37.8) | 0 | | 0 |
| Anemia | 15 | (33.3) | 6 | (13.3) | 0 |
| Cough | 14 | (31.1) | 0 | | 0 |
| Vomiting | 14 | (31.1) | 1 | (2.2) | 0 |
| Pyrexia | 11 | (24.4) | 0 | | 0 |
| Back pain | 10 | (22.2) | 0 | | 0 |
| Dizziness | 10 | (22.2) | 0 | | 0 |
| Rash | 10 | (22.2) | 0 | | 0 |
| Hypophosphatemia | 9 | (20.0) | 5 | (11.1) | 0 |
| Febrile neutropenia | 3 | (6.7) | 3 | (6.7) | 0 |

*Includes neutropenia and neutrophil count decreased.

Clinical Activity of Sacituzumab Govitecan Overall and in Patient Subgroups—Overall, 31.1% (14 of 45) of patients achieved objective responses (95% CI, 18.2% to 46.6%; Table 4). Responses included 2 CRs (4.4%) and 12 PRs (26.7%). SD was observed in 35.6% (16 of 45) of patients, and 22.2% (10 of 45) of patients had progressive disease. The CBR (including CR, PR and SD≥6 months) was 46.7% (21 of 45 patients). The median time to objective response was 1.9 months (range, 1.7 to 7.4 months) and the median DOR (duration of response) was 12.9 months (Table 4).

TABLE 4
Summary of Treatment Efficacy (Overall mUC Cohort)

| Variable | Patients (N = 45) | |
|---|---|---|
| CR, n (%) | 2 | (4.4) |
| PR, n (%) | 12 | (26.7) |
| SD, n (%) | 16 | (35.6) |
| PD, n (%) | 10 | (22.2) |
| Not assessed, n (%) | 5 | (11.1) |
| ORR | | |
| No. of patients | 14 | |
| % of patients (95% CI) | 31.1 | (18.2 to 46.6) |
| Clinical benefit rate | | |
| No. of patients | 21 | |
| % of patients (95% CI) | 46.7 | (31.7 to 62.1) |

| | |
|---|---|
| Time to onset of response (months) | |
| Median | 1.9 |
| Range | 1.7-7.4 |
| Median DOR (months) | |
| Median | 12.9 |
| 95% CI | 5.1, not calculable |
| Range | 1.3-29.4+ |

Subgroup analysis of ORR showed a response rate of 33.3% (5 of 15 patients) in patients with liver metastases and 27.3% (9 of 33 patients) in those with any visceral involvement (Table 4). The ORR in patients who were previously treated with CPI (17 of 45 patients) and patients previously treated with both CPI and platinum therapies (15 of 45 patients) were 23.5% (4 of 17) and 26.7% (4 of 15) of patients, respectively.

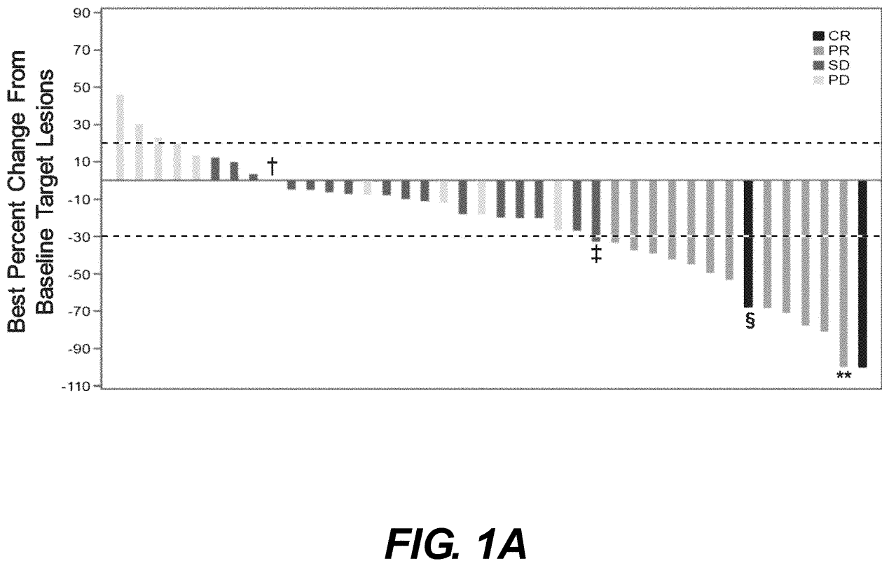

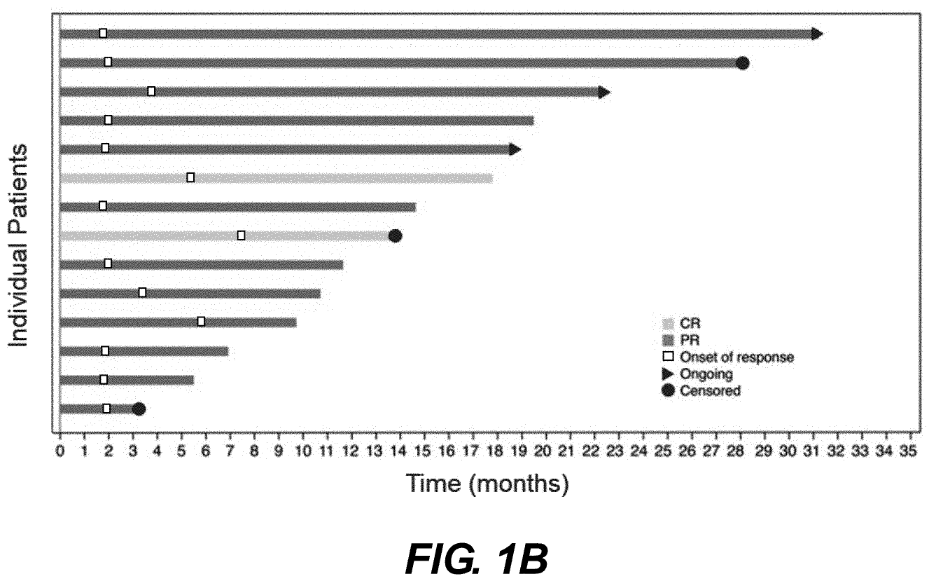

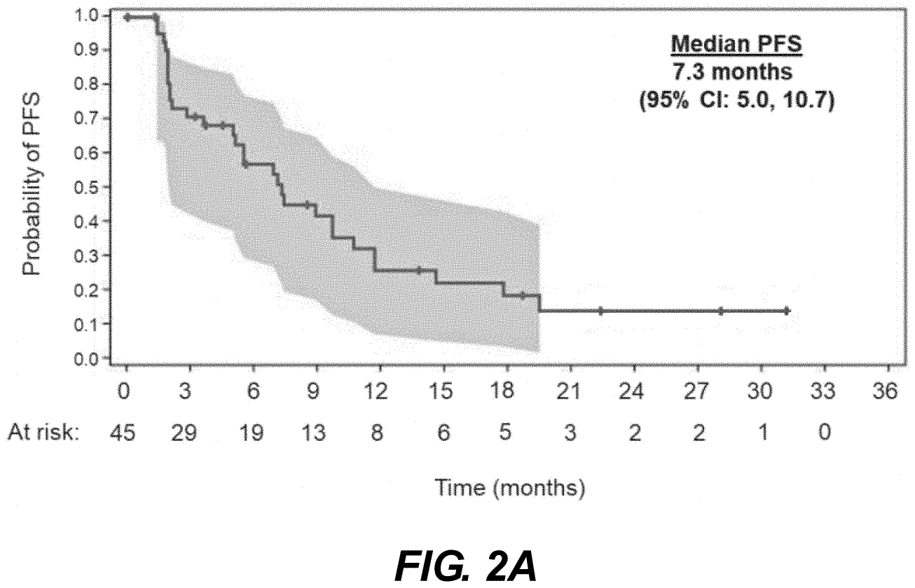

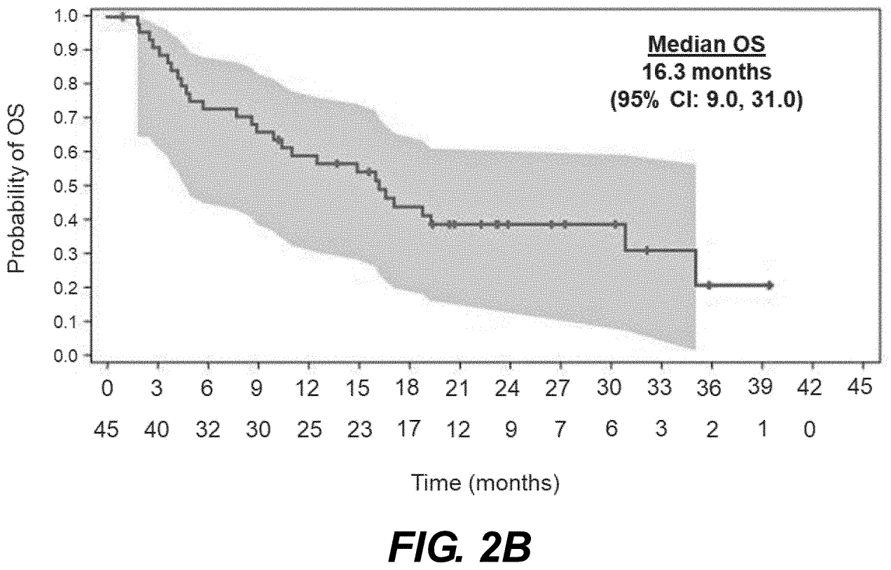

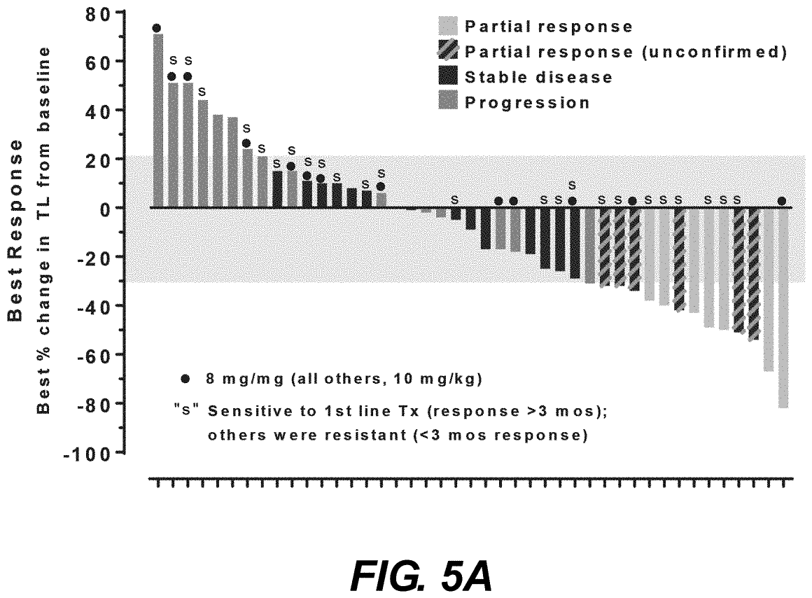

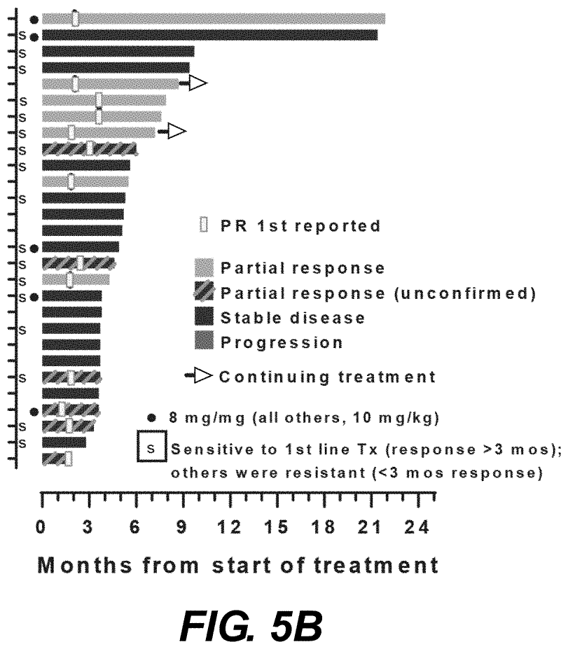

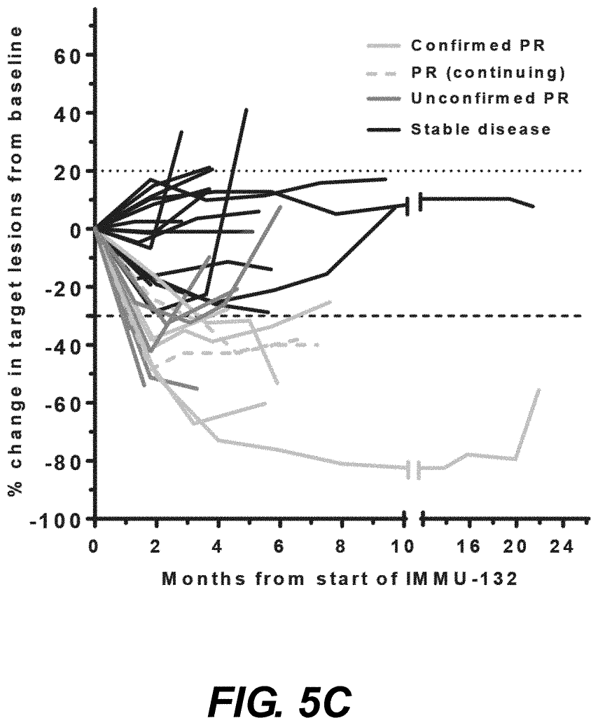

A reduction of target lesions was achieved by 77.5% of patients (31 of 40 patients with at least one post-baseline tumor assessment; FIG. 1A). Fifty percent of responders (7 of 14) had a response lasting more than 12 months. At the time of this analysis, 3 patients with ongoing response were still on treatment (FIG. 1B) and 5 patients remained on treatment at the time of data cut-off. The median PFS and median OS were 7.3 months (95% CI, 5.0 to 10.7 months) and 16.3 months (95% CI, 9.0 to 31.0 months), respectively (FIG. 2).

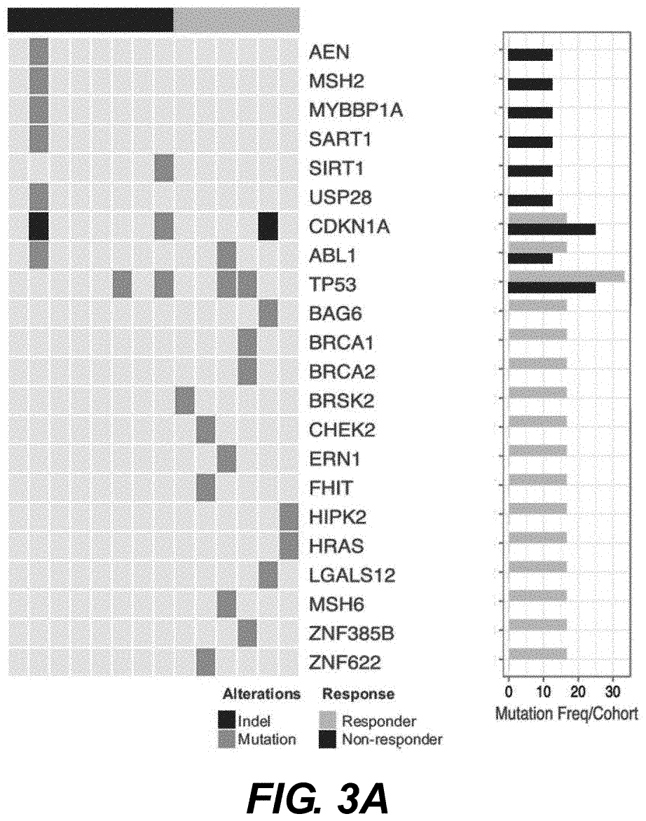

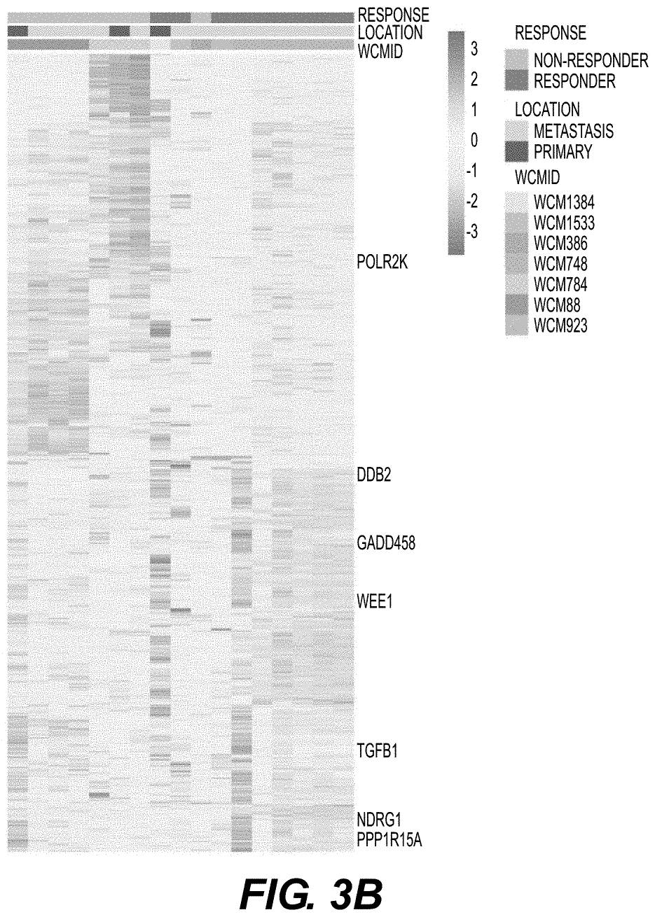

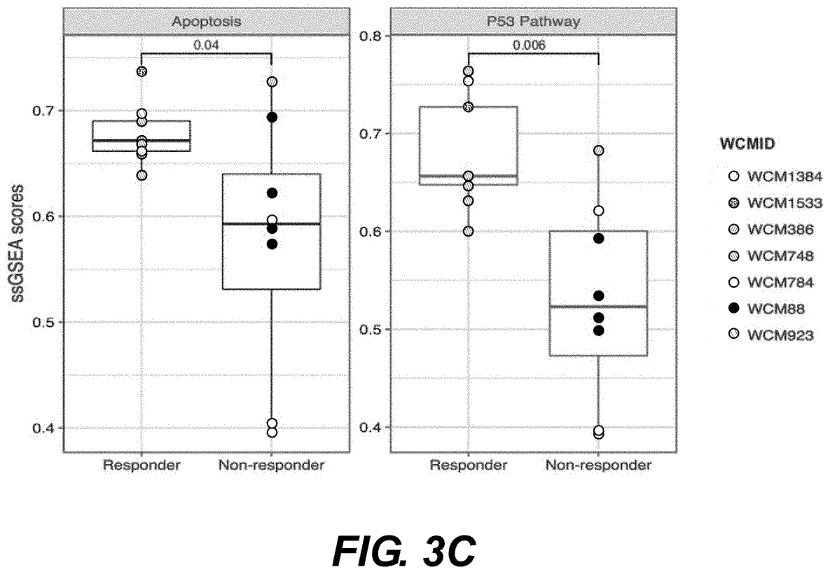

Genomic Assessments—Results of the WES and RNAseq analyses showed that mutations in the intrinsic apoptotic signaling pathway (G0:0097193), which includes DNA damage repair and apoptosis genes, were enriched in responders compared with non-responders (unadjusted p=0.02). Several DNA damage response and repair (BRCA1, BRCA2, CHEK2, MSH2, MSH6, TP53, CDKN1A) and apoptosis (BAG6, BRSK2, ERN1, FHIT, HIPK2, LGALS12, ZNF622, AEN, SART1, USP28) genes in this pathway were differentially mutated between the two groups (FIG. 3A). Analysis of the RNAseq data identified the GADD45B, TGFB1, NRG1, WEE1, and PPP1R15A genes among the top differentially regulated genes between responders and non-responders to sacituzumab govitecan (FIG. 3B). These genes are functionally linked to response to irinotecan or its metabolite, SN-38 (Miettinen et al., 2009, Anticancer Drugs 20:589-600; Bauer et al., 2012, PLoS One 7:e39381; Yang et al., 2017, Oncotarget 8:47709-24; Yin et al., 2018, Mol Med Rep 17:3344-49; Roh et al., 2016, J Cancer Res Clin Oncol 142:1705-14). Results of the single-sample GSEA analyses showed an enrichment of differential changes in the apoptosis pathway (p=0.04) and the p53 pathway (p=0.006) in responders to sacituzumab govitecan (FIG. 3C), which is consistent with the role of p53 signaling in mediating the downstream cytotoxic effects of SN-38 te Poele & Joele, 1999, Br J Cancer 81:1285-93).

Specific data on genomic biomarkers, allele frequencies and specific mutations or other genetic variations is disclosed in Appendix 1 and Appendix 2. Appendix 1 identifies the specific genomic biomarkers identified in mUC patient samples. Column 1 lists the genes in which the biomarker occurred, the chromosome number, start position and end position of the genetic variant, the type of variant, where appropriate (e.g., SNP) the reference allele and tumor allele, resulting changes in codon and protein sequences and the tumor VAF (variant allele frequency).

Appendix 2, part A segregates the responder and nonresponder mutation frequencies for each gene mutated, with the gene identified in column 1, followed by responder mutation frequency, nonresponder mutation frequency and presence or absence in samples from each patient. The specific type of genetic variation (SNP or insertion/deletion) is also indicated. Appendix 2, part B, lists the individual genes examined and the biomarkers observed in responders vs. non-responders. Appendix 2, part C summarizes the GSEA scores for the P53 and apoptosis pathways for each sample, categorized as responders or nonresponders to sacituzumab govitecan.

### Discussion

Patients with mUC who have disease progression after chemotherapy and CPIs have poor outcomes and no approved treatment options (Di Lorenzo et al., 2015, Medicine (Baltimore) 94:e2297; Vlachostergios et al., 2018, Bladder Cancer 4:247-59). Developing safe and effective treatments for these patients is critical, and ADCs represent a promising therapeutic modality (Vlachostergios et al., 2018, Bladder Cancer 4:247-59; Starodub et al., 2015, Clin Cancer Res 21:3870-8; Rosenberg et al., 2019, J Clin Oncol 37(suppl 7S):377). Our study shows that sacituzumab govitecan has significant clinical activity in this heavily pretreated population of patients with resistant/refractory mUC, achieving an objective response of 31%, including a 33% response rate in those with liver metastases. Although patients with prior CPI exposure had poorer performance status and more lines of therapy, 23.5% had objective response. Overall, patients achieved durable clinical benefit, with a median DOR of 12.9 months and 50% of responders having response duration of more than 12 months, up to the longest ongoing response at 29.4 months at the time of data cutoff. Despite receiving a median of 2 prior lines of therapy, the median PFS and OS observed with sacituzumab govitecan were 7.3 months and 16.3 months, respectively, with 5 patients continuing to receive treatment at the time of data cut-off. These early results for sacituzumab govitecan in mUC report a median OS that is longer than that observed with other standard-of-care or investigative treatments (range: 4.3 to 13.8 months) in similar second-line settings in mUC patients (Bellmunt et al., 2017, N Eng J Med 37:1015-26; Patel et al., 2018, Lancet Oncol 19:51-64; Rosenberg et al., 2016, Lancet 387:1909-20; Rosenberg et al., 2019, J Clin Oncol 37(suppl 7S):377; Bellmunt et al., J Clin Oncol 27:4454-61; Bellmunt et al., 2013, Ann Oncol 24:1466-72; Siefker-Radtke et al., 2018, J Clin Oncol 36:4503). Collectively, these findings suggest that sacituzumab govitecan is effective in patients with resistant/refractory mUC.

The AEs associated with sacituzumab govitecan were predictable and manageable, resulting in a low rate of discontinuation. The safety profile was consistent with that reported for sacituzumab govitecan in other cancers (Starodub et al., 2015, Clin Cancer Res 21:3870-8; Ocean et al., 2017, Cancer 123:3843-54; Bardia et al., 2019, N Engl J Med 380:741-51; Gray et al., 2017, Clin Cancer Res 23:5711-19; Heist et al., 2017, J Clin Oncol 35:2790-97). Severe diarrhea is a major concern with irinotecan (Rothenberg, 1997, Ann Oncol 8:837-55; Beer et al., 2008, Clin Genitourin Cancer 6:36-9; Camptosar [package insert] New York, N.Y., Pharmacia & Upjohn, 2016), with a 31% rate of grade ≥3 events of late diarrhea and an 8% rate of grade ≥3 events of early diarrhea reported with irinotecan administered as single-agent therapy (Camptosar [package insert] New York, N.Y., Pharmacia & Upjohn, 2016). Notably, the incidence of grade 3 diarrhea observed with sacituzumab govitecan in this study was low (9%), with no cases of grade 4 or higher diarrhea and only one treatment discontinuation due to diarrhea. Despite the expression of Trop-2 in normal tissues (Trerotola et al., 2013, Oncogene 32:222-33; Goldenberg et al., 2018, Oncotarget 9:28989-29006), sacituzumab govitecan toxicity, including frequent myelosuppression, was manageable with dosing schedule modification and supportive care, ensuring a >90% relative dose intensity and a low rate of treatment discontinuations due to AEs. In fact, in our study there were no treatment discontinuations due to neutropenia and a high response rate was reported, despite 40% of patients having dose reductions. Consistent with what has been reported in other populations treated with sacituzumab govitecan (Bardia et al., 2017, J Clin Oncol 35:2141-48; Bardia et al., 2019, N Engl J Med 380:741-51; Bardia et al., 2018, J Clin Oncol 36(suppl):1004; Gray et al., 2017, Clin Cancer Res 23:5711-19; Heist et al., 2017, J Clin Oncol 35:2790-97), there were no cases of grade 3 or higher neuropathy or cardiovascular AEs. Importantly, no treatment-related deaths were reported in our study.

Integrated genomic and transcriptomic analysis in a subset of patients in this study showed a distinct pattern of differential somatic mutations and gene expression in the DNA damage response and apoptosis pathways between responders and non-responders to sacituzumab govitecan. This is consistent with the biological effects of SN-38 in inducing DNA damage and the activation of p53-mediated apoptosis (Candeil et al., 2004, Int J Cancer 109:848-54; Tomicic et al., 2013, Biochim Biophys Acta 1835:11-27). It is notable that the combination of sacituzumab govitecan and poly-ADP-ribose polymerase (PARP) inhibitors in triple negative breast cancer cell lines and mouse xenograft models resulted in enhanced antitumor activity, independent of BRCA1/2 mutation status (Cardillo et al., 2017, Clin Cancer Res 23:3405-15). Taken together, our findings lay the foundation for a deeper understanding of the biological effects of sacituzumab govitecan and, if validated prospectively, may have important implications for selecting patients who are most likely to benefit from treatment.

A major strength of our study is that at least 38% of this population received sacituzumab govitecan as a fourth or later line of treatment, including after progression after CPI treatment, allowing assessment of its activity in heavily pretreated patients. In addition, this population was evaluated in a population that is more representative of those in clinical practice. While the small number of patients in certain clinical subgroups limits the interpretation of data from subgroup analyses, the overall efficacy data support use of sacituzumab govitecan for treatment of metastatic urothelial cancer (mUC).

In summary, sacituzumab govitecan demonstrated clinically meaningful activity, including high response rates, long durations of response and survival benefit, and a manageable safety profile in pretreated patients with treatment-resistant/refractory mUC, including patients who were heavily pretreated. An international, multicenter, open-label, phase II study (TROPHY-U-01, NCT03547973) is underway to further evaluate the efficacy and safety of sacituzumab govitecan in patients with mUC after failure of platinum-based chemotherapy regimens or anti-PD-1/PD-L1 based immunotherapy.

### Example 2. Treatment of Metastatic Triple-Negative Breast Cancer with the Anti-Trop-2 ADC Sacituzumab Govitecan

Triple-negative breast cancer (TNBC) is characterized by the absence of the estrogen receptor, progesterone receptor and HER2 expression. TNBC accounts for approximately 20% of breast cancers and shows a more aggressive clinical course and higher risk of recurrence and death. Because of the absence of hormone receptor targets, there is a lack of appropriate targeted therapies for TNBC (Jin et al., 2017, Cancer Biol Ther 18:369-78), although atezolizumab in combination with abraxane chemotherapy has recently been approved for first line therapy of TNBC. To date, the main systemic treatment for TNBC has been platinum-based chemotherapy, primarily with cisplatin and carboplatin (Jin et al., 2017, Cancer Biol Ther 18:369-78). However, resistance to or relapse from these agents is common. Over 75% of BRCA1/2 mutated breast cancers show the TNBC phenotype, and homologous recombination deficiency (HRD) resulting from the loss of BRCA function due to mutation or methylation has been suggested to be predictive of platinum efficacy (Jin et al., 2017, Cancer Biol Ther 18:369-78). The present study reports the results of a phase I/II clinical trial (NCT01631552) in patients with metastatic TNBC who had previously failed therapy with at least one standard anti-cancer treatment. The results reported below demonstrate the safety and efficacy of sacituzumab govitecan, an anti-Trop-2 ADC, in a heavily pretreated population of metastatic, relapsed/refractory TNBC.

Methods and Materials

Patients with relapsed/refractory TNBC who had previously failed at at least one prior line of therapy were enrolled in a single-arm, multicenter trial (Bardia et al., 2019, N Engl J Med 380:741-51). The present study reports on 108 patients who had failed at least two prior lines of therapy (median three prior therapies) (Bardia et al., 2019, N Engl J Med 380:741-51). Patients received a 10 mg/kg starting dose on days 1 and 8 of a 21 day cycle that was repeated until disease progression or unacceptable adverse events. For severe treatment-related adverse events, a 25% dose reduction was allowed after the first occurrence, 50% after the second and discontinuation after the third. Of the 108 patients, 107 were female and 1 was male, with a median age of 55. Prior therapies included treatment with taxanes (98%), anthracyclines (86%), platinum agents (69%), gemcitabine (55%), eribulin (45%) and checkpoint inhibitors (17%). Tumor staging was performed by computed tomography (CT) and MRI at baseline, followed up at 8 week intervals from the start of treatment until disease progression.

Results

The most common adverse events included nausea (67% of patients, 6% with grade 3), diarrhea (62%, 8% grade 3), vomiting (49%, 6% grade 3), fatigue (55%, 8% grade 3), neutropenia (64%, 26% grade 3), and anemia (50%, 11% grade 3). The only grade 4 adverse events observed were neutropenia (16%), hyperglycemia (1%), and decreased white blood cell count (3%). Four patients died during the course of study. Each of these was attributed by the investigators to disease progression and not to toxicity of sacituzumab govitecan (Bardia et al., 2019, N Engl J Med 380:741-51). Three patients discontinued treatment due to adverse events. At the time of data cutoff, the median duration of follow-up among the 108 patients was 9.7 months. Eight patients were continuing to receive therapy and 100 had discontinued therapy, with 86 discontinuing therapy due to disease progression. Transient changes in laboratory safety values included decreases in blood cell counts and alterations in biochemical values, which generally recovered by the end of treatment.

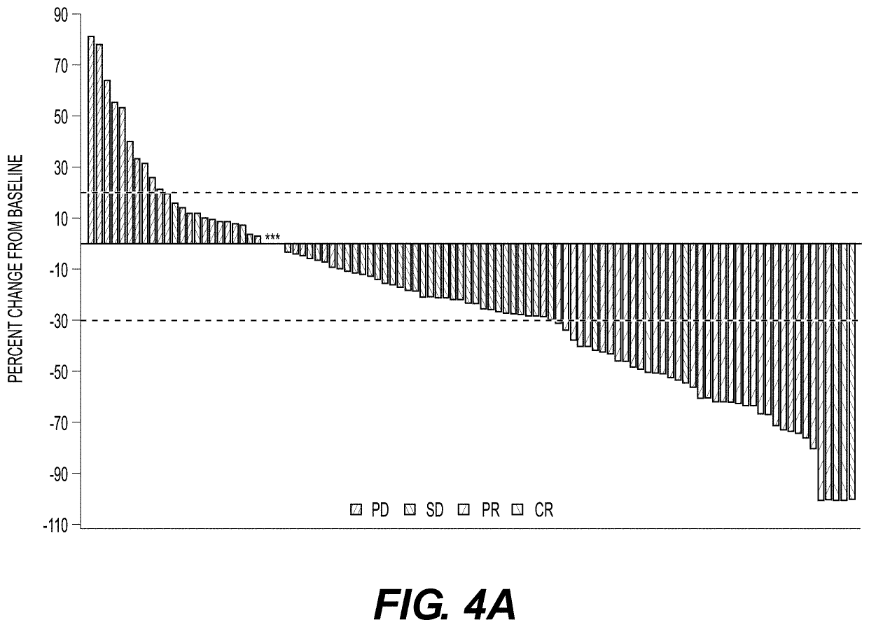

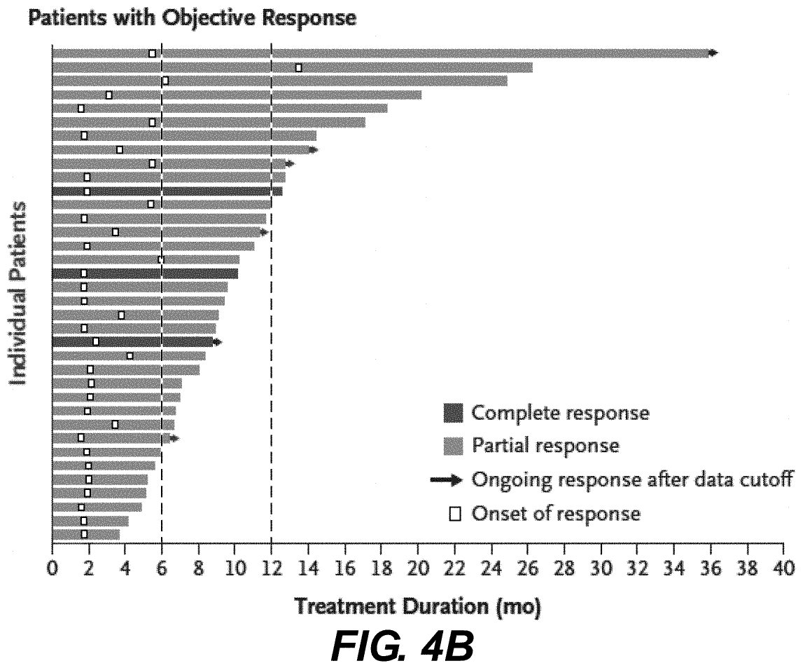

FIG. 4A shows a waterfall plot illustrating the breadth and depth of responses according to local assessment. The response rate (CR+PR) was 33.3%, including 2.8% complete responses (CR). The clinical benefit ratio (including stable disease for at least 6 months) was 45.5%.

FIG. 4B shows a swimmer plot of the onset and durability of response in 36 patients who exhibited an objective response. The median time to response was 2.0 months and median duration of response was 7.7 months. The estimated probability that a patient would exhibit a response was 59.7% at 6 months and 27.0% at 12 months. As of the data cutoff date, 6 patients had long-term responses of more than 12 months. No significant difference in response to sacituzumab govitecan was observed as a function of patient age, onset of metastatic disease, number of previous therapies or the presence of visceral metastases. The response rate was 44% among patients who had failed previous checkpoint inhibitor therapy. Median progression-free survival was 5.5 months and median overall survival was 13.0 months.

### Discussion

The majority of patients with TNBC will progress after receiving first line therapy, and standard therapeutic options are limited to chemotherapy. Chemotherapy is associated with a low response rate (10-15%) and short PFS (2-3 months) in patients with metastatic TNBC who have previously failed standard chemotherapy. Because of the lack of normal breast tissue receptors, there are no present options for targeted therapy of TNBC.

Sacituzumab govitecan (SG) is an anti-Trop-2 ADC, with a humanized RS7 antibody conjugated via a CL2A linker to the topoisomerase I inhibitor, SN-38 (a metabolite of irinotecan). Trop-2 is reported to be expressed in more than 85% of breast cancer tumors (Bardia et al., 2019, N Engl J Med 380:741-51).

The present study shows that in a heavily pretreated population with metastatic, resistant/refractory TNBC, treatment with an optimized dosage of 10 mg/kg of SG resulted in a 33.3% response rate, with a median duration of 7.7 months, median PFS of 5.5 months and median OS of 13.0 months. These numbers are substantially better than the present standard of care in second line or later TNBC patients, which is limited to systemic chemotherapy. Further use of targeted anti-Trop-2 ADCs, alone or in combination with one or more other therapeutic modalities, and with or without use of diagnostic assays to predict which patients are more likely to benefit from monotherapy or combination therapy, will further improve the efficacy of this therapeutic approach for this highly refractory and lethal form of cancer.

### Example 3. Therapy of mSCLC Patients with Anti-Trop-2 ADC

Topotecan, a topoisomerase I inhibitor, is approved as a second-line therapy in patients sensitive to first-line platinum-containing regimens, but only a few new therapeutic agents have been approved for the treatment of metastatic small-cell lung cancer (mSCLC) (Gray et al., 2016, Clin Cancer Res 23:5711-9). In this Example, a novel anti-Trop-2 ADC, sacituzumab govitecan, was studied. Patients with a median of 2 prior therapies (range 1-7) received the ADC on days 1 and 8 of 21-day cycles, with a median of ten doses (range, 1 to 63) being given. The principal grade ≥3 toxicities were manageable neutropenia, fatigue, and diarrhea. Despite up to 63 repeated doses, the ADC was not immunogenic.

Forty-nine percent of the 43 assessable patients had a reduction of tumor size from baseline; the objective response rate (partial responses) was 16% and stable disease was achieved in 49% of patients. Median progression-free survival and median overall survival were 3.6 and 7.0 months, respectively, based on an intention-to-treat (N=53) analysis. This ADC was active in patients who were chemosensitive or chemoresistant to first-line chemotherapy and also in patients who failed second-line topotecan therapy (Gray et al., 2016, Clin Cancer Res 23:5711-9). These data support the use of sacituzumab govitecan as a new therapeutic for advanced mSCLC.

Methods

Patients ≥18 years of age with mSCLC who had relapsed or were refractory to at least one prior standard line of therapy for stage IV metastatic disease, and with measurable tumors by CT, were enrolled. They were required to have Eastern Cooperative Oncology Group (ECOG) performance status of 0 or 1, adequate bone marrow, hepatic and renal function, and other eligibility as described in the phase I trial (Starodub et al., 2015, Clin Cancer Res 21:3870-8). Previous therapy had to be completed at least 4 weeks before enrollment.

The overall objective of this portion of the basket trial being conducted for diverse cancers (ClinicalTrials.gov, NCT01631552) was to evaluate safety and antitumor activity of sacituzumab govitecan in patients with mSCLC. Doses of 8 or 10 mg/kg were given on days 1 and 8 of a 21-day cycle, with contingencies to delay (maximum of 2 weeks). Toxicities were managed by supportive hematopoietic growth-factor therapy for blood cell reduction, dose delays and/or modification as specified in the protocol (e.g., 25% of prior dose), or by standard medical practice. Treatment was continued until disease progression, initiation of alternative anticancer therapy, unacceptable toxicity, or withdrawal of consent.

Fifty-three patients were enrolled with mSCLC (30 females, 23 males, with a median age 63 years (range, 44-82). The median time from initial diagnosis to treatment with sacituzumab govitecan was 9.5 months (range, 3 to 53). Most patients were heavily pretreated, with a median of 2 prior lines of therapy (range, 1 to 7). Everyone had received cisplatin or carboplatin plus etoposide. Twenty-two (41%) patients had 1 prior line of therapy, while 14 (26%) and 17 (32%) were given 2 and ≥3 prior chemotherapy regimens, respectively. In addition, 18 (33%) received topotecan and/or irinotecan, 9 (16%) had a taxane, and 5 (9%) had an immune checkpoint inhibitor therapy, comprising nivolumab (N=4) or atezolizumab (N=1).

Based on a duration of response to a platinum-containing frontline therapy greater or less than 3 months, there were 27 (51%) and 26 (49%) chemosensitive and chemoresistant patients, respectively. Most patients had extensive disease, with metastases to multiple organs, including lungs (66%), liver (59%), lymph nodes (76%), chest (34%), adrenals (25%), bone (23%), and pleura (6%). Other sites of disease included pancreas (N=4), brain (N=2), skin (N=2), and esophageal wall, ovary, and sinus (1 each).

The primary endpoint was the proportion of patients with a confirmed objective response, assessed approximately every 8 weeks until disease progression, by each institution's radiology group or a contracted local radiology service. Objective responses were assessed by Response Evaluation Criteria in Solid Tumors, version 1.1 (RECIST 1.1) (Eisenhauer et al., 2009, Eur J Cancer 45:228-47). Partial (PR) or complete responses (CR) required confirmation within 4 to 6 weeks after the initial response. Clinical benefit rate (CBR) is defined as those patients with an objective response plus stable disease (SD) ≥4 months. Survival was monitored every 3 months until death or withdrawal of consent.

Safety evaluations were conducted during scheduled visits or more frequently if warranted. Blood count and serum chemistries were checked routinely before administration of sacituzumab govitecan and when clinically indicated.

Statistical Analyses—The data included in the analyses were derived from patients enrolled from November 2013 to June 2016, with follow-up through Jan. 31, 2017. The frequency and severity of adverse events (AEs) were defined by MedDRA Preferred Term and System Organ Class (SOC) version 10, with severity assessed by NCI-CTCAE v4.03. All patients who received sacituzumab govitecan were evaluated for toxicities.

The protocol provided that objective response rates (ORR) were determined for patients who received ≥2 doses (1 cycle) and had their initial 8-week CT assessment. Duration of response is defined in accordance to RECIST 1.1 criteria, with those having an objective response marked from time of the first evidence of response until progression, while stable disease duration is marked from the start of treatment until progression. PFS and OS were defined from the start of treatment until an objective assessment of progression was determined (PFS) or death (OS). Duration of response, PFS, and OS were estimated by Kaplan-Meier methods, with 95% confidence intervals (CI), using MedCalc Statistical Software, version 16.4.3 (Ostend, Belgium).

Tumor Trop-2 Immunohistochemistry and Immunogenicity of Sacituzumab Govitecan and Components—Archival tumor specimens for Trop-2 were stained by IHC and interpreted as reported previously (Starodub et al., 2015, Clin Cancer Res 21:3870-8). Positivity required at least 10% of the tumor cells to be stained, with an intensity scored as 1+(weak), 2+(moderate), and 3+(strong). Antibody responses to sacituzumab govitecan, the IgG antibody, and SN-38 were monitored in serum samples taken at baseline and then prior to each even-numbered cycle by enzyme-linked immunosorbent assays performed by the sponsor (Starodub et al., 2015, Clin Cancer Res 21:3870-8). Assay sensitivity is 50 ng/mL for the ADC and the IgG, and 170 ng/mL for anti-SN-38 antibody.

Results

Patients—From November 2013 to June 2016, 53 patients were enrolled with mSCLC (30 females, 23 males, with a median age 63 years (range, 44-82). The median time from initial diagnosis to treatment with sacituzumab govitecan was 9.5 months (range, 3 to 53). Most patients were heavily pretreated, with a median of 2 prior lines of therapy (range, 1 to 7). Everyone received cisplatin or carboplatin plus etoposide. Twenty-two (41%) patients had 1 prior line of therapy, while 14 (26%) and 17 (32%) were given 2 and ≥3 prior chemotherapy regimens, respectively. In addition, 18 (33%) received topotecan and/or irinotecan, 9 (16%) had a taxane, and 5 (9%) had an immune checkpoint inhibitor therapy, comprising nivolumab (N=4) or atezolizumab (N=1). Most patients had extensive disease, with metastases to multiple organs, including lungs (66%), liver (59%), lymph nodes (76%), chest (34%), adrenals (25%), bone (23%), and pleura (6%). Other sites of disease included pancreas (N=4), brain (N=2), skin (N=2), and esophageal wall, ovary, and sinus (1 each).

Treatment Exposure, Safety and Tolerability—Of the 53 patients enrolled, two first treated in May 2016 were continuing sacituzumab govitecan therapy at the cutoff date of Jan. 31, 2017. All other patients had discontinued treatment and otherwise were being monitored for survival. More than 590 doses (over 295 cycles) have been administered, with a median of 10 doses (range, 1-63) per patient. No infusion-related reactions were reported.

The initial doses in 15 patients were given at a starting dose of 8 mg/kg; 10 mg/kg was the starting dose for the next 38 patients. Between the 2 dose groups, 25 patients received ≥10 doses (≥5 cycles), and 2 received 62 and 63 doses (>30 cycles). The median treatment duration was 2.5 months (range, 1 to 23). Neutropenia (grade ≥2) was the only indication for dose reduction and was recorded in 29% (11/38) patients at the 10 mg/kg dose level after a median of 2.5 doses (range, 1 to 9). Two of the fifteen patients (13%) treated at 8 mg/kg had reductions, one after 2 doses and another after 41 doses (20 cycles). Once reduced, additional reductions were infrequent. No treatment-related deaths were observed.

In this trial, ten patients dropped out before the first response assessment; four received 1 dose, five received 2 doses, and another after 4 doses. Three were ineligible for response evaluation after receiving 1 or 2 doses, because one had mixed histology of SCLC and NSCLC, and the other 2 were diagnosed with pre-trial brain and/or spinal cord metastases after receiving the first dose of sacituzumab govitecan. Two patients who reported CTCAE grade 3 adverse events (neutropenia and fatigue) after one dose that did not recover in time for the second dose were discontinued per protocol guidelines. Four patients withdrew from the study after 2 doses, 2 withdrew consent and 2 withdrew due to grade 2 fatigue. An additional patient left the study after 4 treatments because of concurrent multiple comorbidities, dying suddenly before the first response assessment.

The most frequently reported AEs in the 53 patients receiving at least one dose of sacituzumab govitecan were nausea, diarrhea, fatigue, alopecia, neutropenia, vomiting and anemia (data not shown). Grade 3 or 4 neutropenia occurred in 34% (18/53) of patients, and only one patient had febrile neutropenia. Other grade 3 or 4 adverse events were few, and included fatigue (13%), diarrhea (9%), anemia (8%), increased alkaline phosphatase (8%), and hyponatremia (8%). While there were fewer patients requiring dose reduction in the 8 mg/kg dose group (13% vs 28% in 10 mg/kg), the 10 mg/kg dose level was equally well tolerated, with dose modification and/or growth factor support in a few patients.

Efficacy—As described, of the 53 mNSCLC patients enrolled, ten discontinued prior to their first CT response assessment, leaving 43 patients with the protocol-required objective assessment of response after receiving at least two doses of sacituzumab govitecan and at least one follow-up scan. FIG. 5 provides a series of graphic representations of the responses, including a waterfall plot of the best percentage change in the diameter sum of the target lesions for the 43 patients (FIG. 5A), a graph showing the duration of the responses for those achieving PR or SD status (FIG. 5B), and a plot tracking the response changes of the patients with PR and SD over time (FIG. 5C).

Twenty-one of the 43 CT-assessable patients (49%) experienced a reduction of tumor size from baseline (FIG. 5A). Confirmed partial responses (≥30% reduction) occurred in seven patients, yielding an ORR of 16% (Table 5). The median time to response in these patients was 2.0 months (range, 1.8 to 3.6 months), with a Kaplan-Meier estimated median duration of response of 5.7 months (95% CI: 3.6, 19.9). Two of the seven responders had ongoing responses at the last follow-up (i.e., patients were alive, free of disease progression, and had not started alternate anticancer treatments), one at 7.2+ months and the other 8.7+ months from start of treatment.

TABLE 5
Response summary of sacituzumab govitecan (SG) in SCLC patients

| Best overall response, N (%) | | |
|---|---|---|
| Total with response assessment | 43 | |
| PR (confirmed) | 7 | (16%) |
| PRu (unconfirmed; SD with >30% shrinkage as best response) | 6 | (14%) |
| SD | 15 | (35%) |
| PD | 15 | (35%) |
| Clinical benefit rate (PR + SD ≥ 4 months) N (%) | 17/43 | (40%) |
| Duration of confirmed objective response, months median (95% CI) | 5.7 | (3.6, 19.9) |
| Progression-free survival, months (N = 53), median (95% CI) | 3.6 | (2.0, 4.3) |
| Overall survival, months (N = 53), median (95% CI) | 7.0 | (5.5, 8.3) |
| SG response assessment in patients who were sensitive | | |
| (N = 24) to 1^{st} line. | | |
| PFS (median months; 95% CI) | 3.8 | (2.8, 6.0) |
| OS (median months; 95% CI) | 8.3 | (7.0, 13.2) |
| Clinical benefit rate (PR + SD ≥ 4 months) N (%) | 12/24 | (50%) |
| SG response assessment in patients who were resistant | | |
| (N = 19) to 1^{st} line. | | |
| PFS (median months; 95% CI) | 3.6 | (1.8, 3.8) |
| OS (median months; 95% CI) | 6.2 | (4.0, 10.5) |
| Clinical benefit rate (PR + SD ≥ 4 months) N (%) | 5/19 | (26%) |
| Patients receiving SG as second line (N = 19) | | |
| PFS, median months (95% CI) | 3.6 | (2.0, 5.3) |
| OS (median months; 95% CI) | 8.1 | (7.5, 10.5) |
| Clinical benefit rate (PR + SD ≥ 4 months) N (%) | 7/19 | (37%) |
| Patients receiving SG as ≥3 line (N = 24) | | |
| PFS, median months (95% CI) | 3.7 | (1.8, 5.5) |
| OS (median months; 95% CI) | 7.0 | (6.2, 20.9) |
| Clinical benefit rate (PR + SD ≥ 4 months) N (%) | 9/24 | (38%) |
| SG given as ≥3 line and | | |
| Prior topotecan/irinotecan (N = 15) | | |
| PFS, median months (95% CI) | 3.6 | (3.3, 5.5) |
| OS (median months; 95% CI) | 8.8 | (6.2, 20.9) |
| Clinical benefit rate (PR + SD ≥ 4 months) N (%) | 6/15 | (40%) |
| No prior topotecan/irinotecan (N = 9) | | |
| PFS, median months (95% CI) | 3.7 | (1.7, 4.3) |
| OS (median months; 95% CI) | 5.5 | (3.2, 8.3) |
| Clinical benefit rate (PR + SD ≥ 4 months) N (%) | 3/9 | (33%) |

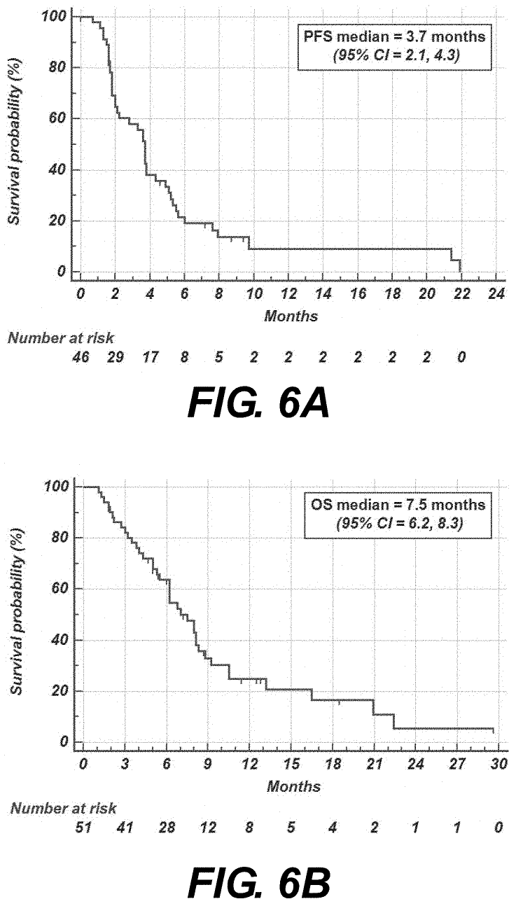

Stable disease (SD) was determined in 21 patients (49%), and included six (14%) who initially had >30% tumor reduction that was not maintained at the subsequent confirmatory CT (unconfirmed PR, or PRu), and three patients who had ≥20% tumor reduction. It is important to note that ten patients had SD for ≥4 months (Kaplan-Meier-derived median=5.6 months, 95% CI: 5.2, 9.7), which was not significantly different from the median PFS for the confirmed PR group (7.9 months, 95% CI: 7.6, 21.9; P=0.1620), and a clinical benefit rate (CBR: PR+SD≥4 months) of 40% (17/43). Indeed, even the OS for these ten SD patients was not significantly different from the seven confirmed PR patients (8.3 months, 95% CI-7.5, 22.4 months vs 9.2 months, 95% CI: 6.2, 20.9, respectively; P=0.5599). This suggests that maintaining SD for a suitable duration (≥4 months) should be an endpoint of interest. On an intention-to-treat (ITT) basis (N=53), the median PFS was 3.6 months (95% CI: 2.0, 4.3) (FIG. 6A), while the median OS was 7.0 months (95% CI: 5.5, 8.3), with 17 patients alive and 5 lost to follow-up (one after 1.8 months, one after 5 months, and three after 11.4-12.8 months) (FIG. 6B).

Thirteen of the 43 patients with an objective response assessment were treated at 8 mg/kg, with one confirmed (8%), one unconfirmed PR, and three SD. In the 10 mg/kg group (N=30), six patients had confirmed PR (20%) and twelve had SD, including five with one CT showing a reduction >30% (PRu). The CBR was 47% (14/30), suggesting that the starting dose of 10 mg/kg provided a better overall response.

Twenty-four patients with a response assessment were classified as sensitive to the first line of platinum-based chemotherapy. Four (17%) achieved a confirmed PR and nine had SD, including four with a single scan showing a >30% tumor reduction (PRu). Nineteen patients were resistant, with three (16%) having confirmed PR and six with SD, including two with PRu. The median PFS for the chemosensitive and chemoresistant groups was 3.8 months (95% CI: 2.8, 6.0) and 3.6 months (95% CI: 1.8, 3.8), respectively, while the median OS was 8.3 months (95% CI: 7.0, 13.2) and 6.2 months (95% CI: 4.0, 10.5), respectively (Table 5). No significant differences in PFS or OS were found between the chemosensitive and chemoresistant groups (P=0.3981 and P=0.3100, respectively).

Nineteen of the 43 patients received sacituzumab govitecan in the second-line setting, and 3/19 (16%) had a PR and seven SD as best response (two of the latter had one >30% tumor shrinkage). The response seen in these patients was the same as that found for the patients who were given sacituzumab govitecan as their third or higher line of therapy (N=24), with four confirmed PR (16%) and 8 SD, including four SD patients with >30% tumor shrinkage on one CT. No significant differences in duration of the PFS or OS were found (P=0.9538 and P=0.6853, respectively). Response analyses are summarized in Table 5.

Among the five patients who received prior treatment with an immune checkpoint inhibitor (CPI), one experienced an unconfirmed PR (54% shrinkage on first assessment, withdrew consent without additional treatment or assessments), two achieved SD with one having 17% tumor shrinkage lasting 8.7 months and the other no change in tumor size for 3.7 months, one had progressing disease, while the fifth patient withdrew consent after one cycle of sacituzumab govitecan. All of the CPI-treated patients either failed to respond to the CPI or progressed before receiving sacituzumab govitecan, indicating that patients can be responsive to sacituzumab govitecan after receiving CPI-treatment.

Of the 24 patients who received sacituzumab govitecan as third- or later-line therapy, fifteen had previously received topotecan and/or irinotecan, while nine never received these agents. The objective responses in these two groups were similar, with no significant difference in PFS (3.8 vs 3.7 months; P=0.7341). However, those treated with sacituzumab govitecan who received prior topotecan therapy had a significantly longer OS than those who did not (8.8 months, 95% CI: 6.2, 20.9 vs 5.5 months, 95% CI: 3.2, 8.3; P=0.0357). The longer OS in this group may reflect the known activity of topotecan in patients who are platinum-sensitive, and therefore may have a better long-term outcome.

Immunohistochemical (IHC) Staining of Tumor Specimens—Archival tumor specimens were obtained from 29 patients, but four were inadequate for review, leaving 25 assessable tumors, of which 92% were positive, with two (8%) having strong (3+) and thirteen (52%) moderate (2+) staining. Twenty-three of these patients had an objective response assessment. There were five with confirmed PR and two unconfirmed PR in this group; five had 2+ staining, while the other two were 1+(not shown), suggesting that higher expression provided better responses. However, an assessment of PFS and OS values against IHC score showed no clear trend (not shown), and Kaplan-Meier estimates for PFS and OS for patients with IHC scores of 0 and 1+ combined (N=10) vs 2+ and 3+ combined (N=13) indicated no significant differences (PFS, P=0.2661; OS, P=0.7186) based on IHC score (not shown).

Immunogenicity of ADC, SN-38, or hRS7 Antibody—No neutralizing antibodies to sacituzumab govitecan, the hRS7 antibody, or SN-38 were detected in patients who maintained treatment for even up to 22 months.

### Discussion

The relapse of SCLC to frontline chemotherapy continues to be divided into two categories, resistant relapse, occurring within three months of the first platinum-based therapy, and sensitive relapse, which occurs after at least 3 months post treatment (O'Brien et al., 2006, J Clin Oncol 24:5441-7; Perez-Soler et al., 1996, J Clin Oncol 14:2785-90). Although there is still some ambiguity regarding the best management of recurrent SCLC, topotecan, a topoisomerase-I inhibitor similar to the SN-38 used in the ADC studied here, is the only product approved for chemosensitive relapse, as supported by numerous trials (O'Brien et al., 2006, J Clin Oncol 24:5441-7; Horita et al., 2015, Sci Rep 5:15437). However, the efficacy and adverse events of topotecan have varied considerably in prior studies, as demonstrated in a meta-analysis of over a thousand patients reported in 14 articles that topotecan had an objective response rate of 5% in chemoresistant frontline patients and 17% in chemosensitive patients (Horita et al., 2015, Sci Rep 5:15437). There were grade ≥3 neutropenia, thrombocytopenia, and anemia in 69%, 1%, and 24% of patients, respectively, and approximately 2% of patients died from this chemotherapy (Horita et al., 2015, Sci Rep 5:15437). Thus, topotecan shows some promise in this second-line setting in patients who relapsed after showing sensitivity to a platinum-based chemotherapy, but with considerable hematological toxicity. However, even this conclusion was challenged recently by Lara et al. (2015, J Thorac Oncol 10:110-5), who asserted that platinum-sensitivity is not strongly associated with improved PFS and OS following treatment with topotecan, which is its currently approved indication.

It is in this setting that the results reported here with sacituzumab govitecan in extended, advanced-disease patients (stage IV) following a median of 2 (range, 1 to 7) prior therapies are promising. Forty-nine percent of patients showed a reduction of tumor measurements from baseline, according to RECIST 1.1, with an ORR of 16% and a median duration of response of 5.7 months (95% CI: 3.6, 19.9). Stable disease was found in 35% of patients, where 14% of these SD patients had >30% tumor shrinkage as best response, although not maintained on the second scan. The clinical benefit rate at ≥4 months was 40%. Median PFS and OS were 3.6 and 7.0 months, respectively. It is interesting that the median OS for the ten patients with SD was 8.3 months (95% CI: 7.5, 22.4), which is not statistically different from the median OS of 9.2 months (95% CI: 6.2, 20.9) for patients with a PR (P=0.5599). In the group receiving 10 mg/kg as their starting dose (N=30), there was a confirmed objective response in six (20%), with an additional five patients having a single CT showing ≥30% tumor reduction (PRu). Also, the clinical benefit rate for this group at the 10 mg/kg dose was 47%. This supports the preferred dose of 10 mg/kg. Noteworthy also is the lack of patient selection required based on immunohistochemical staining of tumor Trop-2, although there was a suggestion that stronger staining correlated with better response, but no significant difference in PFS or OS was found with regard to IHC score.

As mentioned, PFS and OS did not differ substantially between patients with SD >4 months or PR. Patients with unconfirmed PR (i.e., >30% tumor reduction on one CT) or with SD generally are not considered in most ORR assessments. However, the results here indicate no difference in duration of response between patients with confirmed PR or SD lasting for more than 4 months. Indeed, the dynamic tracking of the individual patient responses for PR or SD (especially when the SD last ≥4 months, which is a similar time frame for confirming PR) suggests a clinical benefit for both groups by remaining below the baseline tumor size for several months. Although there was a trend for the PFS of patients with confirmed PR to be longer than the group of patients with SD lasting ≥4 months (P=0.1620), the OS for these 2 groups was not significantly different (P=0.5599). Therefore, while the number of patients in this initial analysis is relatively small, the data suggest that more consideration should be given to disease stabilization as an important indicator of clinical activity when an appropriate duration is achieved, similar to follow-up for patients receiving immune checkpoint inhibitors.

Evaluating patients based on prior chemosensitivity (N=24) or chemoresistance (N=19) shows no response differences with sacituzumab govitecan treatment (Table 5). PFS and OS results were 3.8 and 8.3 months for patients who were chemosensitive in first-line, compared to a PFS and OS of 3.6 months and 6.2 months, respectively, for the chemoresistant group. With no statistical difference, it appears that sacituzumab govitecan can be administered to patients in second- or later-line therapies irrespective of the patients being chemosensitive or chemoresistant to first-line chemotherapy. This differs from topotecan, which is indicated only in those SCFC patients who showed a ≥3-month response to first-line cisplatin and etoposide chemotherapy (O'Brien et al., 2006, J Clin Oncol 24:5441-7; Perez-Soler et al., 1996, J Clin Oncol 14:2785-90). Of 28 patients studied by Perez-Solar et al. (1996, J Clin Oncol 14:2785-90), 11% had a PR, with a median survival of 5 months and a one-year survival of 3.5%.

Although both topotecan and SN-38 are inhibitors of the DNA topoisomerase I enzyme, which is responsible for relaxing a supercoiled DNA helix when DNA is synthesized by stabilizing the DNA complex, causing accumulation of single strand DNA breaks (Takimoto & Arbuck, 1966, Camptothecins. In: Chabner & Fong (Eds.). Cancer Chemotherapy and Biotherapy. Second ed. Philadelphia: Fippincott-Raven; p. 463-84), sacituzumab govitecan showed activity in patients who relapsed after topotecan therapy. Thus, topotecan resistance or relapse may not be a contraindication for administering sacituzumab govitecan, and because of being similarly active in patients who were chemoresistant to cisplatin and etoposide, may be of particular value as a second-line therapeutic in patients with metastatic SCFC regardless of chemosensitivity status.

In the twenty years since the approval of topotecan in the second-line setting, no new agent has been licensed for metastatic SCFC therapy in second-line or later therapy. However, there has been progress more recently with inhibitors of the T-cell checkpoint receptors programmed cell-death protein (PD-1) and cytotoxic T-lymphocyte-associated protein 4 (CTFA-4) (Antonia et al., 2016, Lancet Oncol 17:883-95). These authors conducted a phase I-II trial of nivolumab with or without CTFA-4 antibody ipilimumab in patients with recurrent SCFC. Nivolumab alone achieved a 10% response rate, while the combination had response rates of 19 to 23%, and a disease-control rate of 32% (Antonia et al., 2016, Lancet Oncol 17:883-95). However, a recent study of ipilimumab with or without chemotherapy in SCFC failed to confirm these results (Reck et al., 2016, J Clin Oncol 34:3740-48). Since we observed that sacituzumab govitecan may have activity in patients failing therapy with immune checkpoint inhibitors, we are studying this further, especially because of evidence showing such responses after therapy with an immune checkpoint inhibitor in patients with other cancer types (Bardia et al., 2017, J Clin Oncol 35:2141-48; Faltas et al., 2016, Clin Genitourin Cancer 14:e75-9; Gray et al., 2017, Clin Cancer Res 23:5711-19; Heist et al., 2017, J Clin Oncol 35:2790-97; Tagawa et al., 2017, J Clin Oncol 35:abstract 327; Han et al., 2018, Gynecol Oncol Rep 25:37-40).

Despite recent progress in immunotherapy and the identification of other novel targets for SCLC (Rudin et al., 2017, Lancet Oncol 18:42-51), this still is a lethal disease, especially in the population that is chemoresistant to first-line therapy. The current results of sacituzumab govitecan in heavily-pretreated patients with advanced, relapsed, stage IV, SCLC suggest that this anti-Trop-2 ADC is of use in the therapy of both chemosensitive and chemoresistant SCLC patients, both before or after topotecan.

### Example 4. Clinical Trials with Sacituzumab Govitecan in a Variety of Epithelial Cancers

The present Example reports results from a phase I clinical trial and ongoing phase II extension with sacituzumab govitecan, an ADC of the internalizing, humanized, hRS7 anti-Trop-2 antibody conjugated by a pH-sensitive linker to SN-38 (mean drug-antibody ratio=7.6). Trop-2 is a type I transmembrane, calcium-transducing, protein expressed at high density (˜1×10⁵), frequency, and specificity by many human carcinomas, with limited normal tissue expression. Preclinical studies in nude mice bearing Capan-1 human pancreatic tumor xenografts have revealed sacituzumab govitecan is capable of delivering as much as 120-fold more SN-38 to tumor than derived from a maximally tolerated irinotecan therapy.

The present Example reports the initial Phase I trial of 25 patients (pts) who had failed multiple prior therapies (some including topoisomcrasc-I/II inhibiting drugs), and the ongoing Phase II extension now reporting on 69 pts, including in colorectal (CRC), small-cell and non-small cell lung (SCLC, NSCLC, respectively), triple-negative breast (TNBC), pancreatic (PDC), esophageal, gastric, prostate, ovarian, renal, urinary bladder, head/neck and hepatocellular cancers. Patients were refractory/relapsed after standard treatment regimens for metastatic cancer.

As discussed in detail below, Trop-2 was not detected in serum, but was strongly expressed (≥2⁺) in most archived tumors. In a 3+3 trial design, sacituzumab govitecan was given on days 1 and 8 in repeated 21-day cycles, starting at 8 mg/kg/dose, then 12 and 18 mg/kg before dose-limiting neutropenia. To optimize cumulative treatment with minimal delays, phase II is focusing on 8 and 10 mg/kg (n=30 and 14, respectively). In 49 pts reporting related AE at this time, neutropenia ≥G3 occurred in 28% (4% G4). Most common non-hematological toxicities initially in these pts have been fatigue (55%; ≥G3=9%), nausea (53%; ≥G3=0%), diarrhea (47%; ≥G3=9%), alopecia (40%), and vomiting (32%; ≥G3=2%). Homozygous UGT1A1*28/*28 was found in 6 pts, 2 of whom had more severe hematological and GI toxicities. In the Phase I and the expansion phases, there are now 48 pts (excluding PDC) who are assessable by RECIST/CT for best response. Seven (15%) of the patients had a partial response (PR), including patients with CRC (N=1), TNBC (N=2), SCLC (N=2), NSCLC (N=1), and esophageal cancers (N=1), and another 27 pts (56%) had stable disease (SD), for a total of 38 pts (79%) with disease response; 8 of 13 CT-assessable PDC pts (62%) had SD, with a median time to progression (TTP) of 12.7 wks compared to 8.0 weeks in their last prior therapy. The TTP for the remaining 48 pts is 12.6+ wks (range 6.0 to 51.4 wks). Plasma CEA and CA19-9 correlated with responses. No anti-hRS7 or anti-SN-38 antibodies were detected despite dosing over months. The conjugate cleared from the serum within 3 days, consistent with in vivo animal studies where 50% of the SN-38 was released daily, with >95% of the SN-38 in the serum being bound to the IgG in a non-glucuronidated form, and at concentrations as much as 100-fold higher than SN-38 reported in patients given irinotecan. These results show that the anti-Trop-2 ADC is therapeutically active in numerous metastatic solid cancers, with manageable diarrhea and neutropenia.

Pharmacokinetics

Two ELISA methods were used to measure the clearance of the IgG (capture with anti-hRS7 idiotype antibody) and the intact conjugate (capture with anti-SN-38 IgG/probe with anti-hRS7 idiotype antibody). SN-38 was measured by HPLC. Total sacituzumab govitecan fraction (intact conjugate) cleared more quickly than the IgG (not shown), reflecting known gradual release of SN-38 from the conjugate. HPLC determination of SN-38 (Unbound and TOTAL) showed >95% the SN-38 in the serum was bound to the IgG. Low concentrations of SN-38G suggest SN-38 bound to the IgG is protected from glucuronidation. Comparison of ELISA for conjugate and SN-38 HPLC revealed both overlap, suggesting that ELISA is a surrogate for monitoring SN-38 clearance.

Clinical Trial Status

A total of 69 patients (including 25 patients in Phase I) with diverse metastatic cancers having a median of 3 prior therapies were reported. Eight patients had clinical progression and withdrew before CT assessment. Thirteen CT-assessable pancreatic cancer patients were separately reported. The median TTP (time to progression) in PDC patients was 11.9 wks (range 2 to 21.4 wks) compared to median 8 wks TTP for the preceding last therapy.

A total of 48 patients with diverse cancers had at least 1 CT-assessment from which Best Response and Time to Progression (TTP) were determined. To summarize the Best Response data, of 8 assessable patients with TNBC (triple-negative breast cancer), there were 2 PR (partial response), 4 SD (stable disease) and 2 PD (progressive disease) for a total response [PR+SD] of 6/8 (75%). For SCLC (small cell lung cancer), of 4 assessable patients there were 2 PR, 0 SD and 2 PD for a total response of 2/4 (50%). For CRC (colorectal cancer), of 18 assessable patients there were 1 PR, 11 SD and 6 PD for a total response of 12/18 (67%). For esophageal cancer, of 4 assessable patients there were 1 PR, 2 SD and 1 PD for a total response of 3/4 (75%). For NSCLC (non-small cell lung cancer), of 5 assessable patients there were 1 PR, 3 SD and 1 PD for a total response of 4/5 (80%). Over all patients treated, of 48 assessable patients there were 7 PR, 27 SD and 14 PD for a total response of 34/48 (71%). These results demonstrate that the anti-TROP-2 ADC (hRS7-SN-38) showed significant clinical efficacy against a wide range of solid tumors in human patients.

The reported side effects of therapy (adverse events) are summarized in Table 6. As apparent from the data of Table 6, the therapeutic efficacy of sacituzumab govitecan was achieved at dosages of ADC showing an acceptably low level of adverse side effects.

TABLE 6
Related Adverse Events Listing for sacituzumab govitecan-01
Criteria: Total ≥ 10% or ≥ Grade 3

N = 47 patients

| | TOTAL | Grade 3 | | Grade 4 |
|---|---|---|---|---|
| Fatigue | 55% | 4 | (9%) | 0 |
| Nausea | 53% | 0 | | 0 |
| Diarrhea | 47% | 4 | (9%) | 0 |
| Neutropenia | 43% | 11 | (24%) | 2 (4%) |
| Alopecia | 40% | — | | — |
| Vomiting | 32% | 1 | (2%) | 0 |
| Anemia | 13% | 2 | (4%) | 0 |
| Dysgeusia | 15% | 0 | | 0 |
| Pyrexia | 13% | 0 | | 0 |
| Abdominal pain | 11% | 0 | | 0 |
| Hypokalemia | 11% | 1 | (2%) | 0 |
| WBC Decrease | 6% | 1 | (2%) | 0 |
| Febrile Neutropenia | 6% | 1 | (2%) | 2 (4%) |
| Deep vein thrombosis | 2% | 1 | (2%) | 0 |
| Grading by CTCAE v 4.0 | | | | |

Exemplary partial responses to the anti-Trop-2 ADC were confirmed by CT data (not shown). As an exemplary PR in CRC, a 62 year-old woman first diagnosed with CRC underwent a primary hemicolectomy. Four months later, she had a hepatic resection for liver metastases and received 7 mos of treatment with FOLFOX and 1 mo 5FU. She presented with multiple lesions primarily in the liver (3+ Trop-2 by immunohistology), entering the sacituzumab govitecan trial at a starting dose of 8 mg/kg about 1 year after initial diagnosis. On her first CT assessment, a PR was achieved, with a 37% reduction in target lesions (not shown). The patient continued treatment, achieving a maximum reduction of 65% decrease after 10 months of treatment (not shown) with decrease in CEA from 781 ng/mF to 26.5 ng/mF), before progressing 3 months later.

A 65 year-old male, diagnosed with stage IIIB NSCFC (squamous cell) served as an exemplary example of PR in NSCFC. Initial treatment of carboplatin/etoposide (3 mo) in concert with 7000 cGy XRT resulted in a response lasting 10 mo. He was then started on erlotinib maintenance therapy, which he continued until he was considered for the sacituzumab govitecan trial, in addition to undergoing a lumbar laminectomy. He received the first dose of sacituzumab govitecan after 5 months of erlotinib, presenting at the time with a 5.6 cm lesion in the right lung with abundant pleural effusion. He had just completed his 6^{th} dose two months later when the first CT showed the primary target lesion reduced to 3.2 cm (not shown).

A 65 year-old woman, diagnosed with poorly differentiated SCFC served as an exemplary example of PR in a patient with SCFC. After receiving carboplatin/etoposide (Topo-II inhibitor) that ended after 2 months with no response, followed with topotecan (Topo-I inhibitor) that ended after 2 months, also with no response, she received local XRT (3000 cGy) that ended 1 month later. However, by the following month progression had continued. The patient started with sacituzumab govitecan the next month (12 mg/kg; reduced to 6.8 mg/kg; Trop-2 expression 3+), and after two months of sacituzumab govitecan, a 38% reduction in target lesions, including a substantial reduction in the main lung lesion occurred (not shown). The patient progressed 3 months later after receiving 12 doses.

These results are significant in that they demonstrate that the anti-Trop-2 ADC was efficacious, even in patients who had failed or progressed after multiple previous therapies.

In conclusion, at the dosages used, the primary toxicity was a manageable neutropenia, with few Grade 3 toxicities. Sacituzumab govitecan showed evidence of activity (PR and durable SD) in relapsed/refractory patients with triple-negative breast cancer, small cell lung cancer, non-small cell lung cancer, colorectal cancer and esophageal cancer, including patients with a previous history of relapsing on topoisomerase-I inhibitor therapy. These results show efficacy of the anti-Trop-2 ADC in a wide range of cancers that are resistant to existing therapies.

### Example 5. Collection and Analysis of Circulating Tumor Cells (CTCs) and cfDNA

CTC cells are collected from the blood of patients with metastatic TNBC. Samples of 7.5 ml whole blood are collected into CELLSAVE™ preservative tubes for CTC capture with the CELLSEARCH® CTC system (Janssen Diagnostics). Samples of 20 ml whole blood are collected into EDTA-tubes and processed to plasma for cfDNA, as disclosed in Page et al. (2013, PLoS One 8:e77963). cfDNA is isolated from 3 ml of plasma using the QIAAMP® Circulating Nucleic Acid Kit (Qiagen) according to the manufacturer's instructions. Single CTCs are isolated using a DEPARRAY™ system and CTC nucleic acids are subject to AMPLI1™ whole genome amplification.

Custom AMPLISEQ™ panels (Fisher) are designed to screen for mutations in the following genes: 53BP1, AKT1, AKT2, AKT3, APE1, ATM, ATR, BARD1, BAP1, BLM, BRAF, BRCA1, BRCA2, BRIP1 (FANCJ), CCND1, CCNE1, CEACAM5, CDKN1, CDK12, CHEK1, CHEK2, CK-19, CSA, CSB, DCLRE1C, DNA2, DSS1, EEPD1, EFHD1, EpCAM, ERCC1, ESR1, EXO1, FAAP24, FANC1, FANCA, FANCC, FANCD1, FANCD2, FANCE, FANCF, FANCM, HER2, HMBS, HR23B, KRT19, KU70, KU80, hMAM, MAGEA1, MAGEA3, MAPK, MGP, MLH1, MRE11, MRN, MSH2, MSH3, MSH6, MUC1-6, NBM, NBS1, NEK NF-κB, P53, PALB2, PARP1, PARP2, PIK3CA, PMS2, PTEN, RAD23B, RAD50, RAD51, RAD51 AP1, RAD51C, RAD51D, RAD52, RAD54, RAF, K-ras, H-ras, N-ras, RBBP8, c-myc, RIF1, RPA1, SCGB2A2, SLFN11, SLX1, SLX4, TMPRSS4, TP53, PROP-2, USP11, VEGF, WEE1, WRN, XAB2, XLF, XPA, XPC, XPD, XPF, XPG, XRCC4 and XRCC7. AMPLISEQ™ reactions are set up using 10 ng WGA DNA or 8 ng cfDNA. Next generation sequencing is performed on an Ion 316™ chip (ThermoFisher) using an ION PERSONAL GENOME MACHINE® (ThermoFisher), as described in Guttery et al. (2015, Clin Chem 61:974-82). Selected mutations are validated by droplet digital PCR using a Bio-Rad QX200™ droplet digital PCR system as described in Hindson et al. (2011, Anal Chem 83:8604-10). Trop-2 expression levels in CTCs are determined by ELISA, using RS7 anti-Trop-2 antibody.

Patients are treated with combination therapy with olaparib (200 to 300 mg twice a day, depending on patient's calculated creatinine clearance) for 21 days and sacituzumab govitecan (10 mg/kg iv on days 1 and 8 of each 21 day cycle).

Patients are divided into responders (CR+PR+SD>6 months) or non-responders to the combination therapy. Correlation of sensitivity to the combination therapy with the biomarker data from CTC and cfDNA, as well as Trop-2 expression, shows that sensitivity to combination therapy with olaparib and SG is positively correlated with Trop-2 expression and with mutations in BRCA1, BRCA2, PTEN, ERCC1 and ATM. These biomarkers are used as positive indicators for future therapy with the combination of PARP inhibitors and sacituzumab govitecan.

### Example 6. Therapy of Relapsed Metastatic Ovarian Cancer with Sacituzumab Govitecan Plus Prexasertib (LY2606368), a CHK1 Inhibitor

A 66-year-old woman with FIGO stage IV ovarian cancer positive for BRCA1 mutation undergoes primary surgery and postoperative paclitaxel and carboplatin (TC). After a 20-month platinum-free interval, an elevated CA125 level and recurrence in the peritoneum is confirmed by CT. Following retreatment with TC, a hypersensitivity reaction occurs to the carboplatin, which is changed to nedaplatin. A complete response is confirmed by CT. After an 8-month PFI, an elevated serum CA125 level and recurrence in the peritoneum and liver are confirmed.

She is then given combination therapy with anti-Trop-2 ADC (sacituzumab govitecan) plus prexasertib, a CHK1 inhibitor. Sacituzumab govitecan is administered at 10 mg/kg on days 1 and 8 of a 28-day cycle, while prexasertib is administered i.v. at 105 mg/m² every 14 days of the 28 day cycle. Except for transient grade 2 neutropenia and some initial diarrhea, she tolerates the therapy well, which is then repeated, after a rest of 2 months, for another course. Radiological examination indicates that she has partial response by RECIST criteria, because the sum of the diameters of the index lesions decrease by 45%. Her general condition also improves, and she returns to almost the same level of activity as prior to her illness.

### Example 7. Cell Surface Expression of Trop-2 in Normal Vs. Cancer Tissues

Trop-2 expression and localization were determined in a series of normal tissue samples and corresponding cancer tissues by immunohistochemistry (IHC). Trop-2 was typically expressed in a smaller proportion of normal tissue samples and at weaker IHC staining intensities compared to corresponding cancer tissues (Table 7). In tumor cells, Trop-2 overexpression was almost exclusively membranous. However, in associated normal tissues, membranous Trop-2 expression was typically weak or not observed.

TABLE 7
Trop-2 Expression in Normal vs. Cancer Tissues

| Moderate IHC Staining | Strong IHC Staining |
|---|---|
| (% of normal vs cancer | (% of normal vs cancer |
| tissue samples) | tissue samples) |
| Ovarian: 0% vs 46%¹ | Ovarian: 0% vs 16%¹ |
| Colorectal: 0% vs 26%² | Colorectal: 0% vs 21%² |
| Gastric: 0% vs 34%³ | Gastric: 0% vs 22%³ |
| Oral: 0% vs 46%⁴ | Oral: 0% vs 12%⁴ |
| Pancreatic: NR* vs 26%⁵ | Pancreatic: 0% vs 29%⁵ |

¹Bignotti E, et al. Eur J Cancer. 2010; 46: 944-953.
²Ohmachi T, et al. Clin Cancer Res. 2006; 12: 3057-3063.
³Mühlmann G, et al. J Clin Pathol. 2009; 62: 152-158.
⁴Fong D, et al. Mod Pathol. 2008; 21: 186-191.
⁵Fong D, et al. Br J Cancer. 2008; 99: 1290-1295.

From the foregoing description, one skilled in the art can easily ascertain the essential characteristics of this invention, and without departing from the spirit and scope thereof, can make various changes and modifications of the invention to adapt it to various usage and conditions without undue experimentation. All patents, patent applications and publications cited herein are incorporated by reference.

1.-2. (canceled)

3. A method of selecting patients to be treated with an anti-Trop-2 antibody-drug conjugate (ADC) comprising:
a) analyzing a sample from a human cancer patient for the presence of one or more cancer biomarkers;
b) detecting one or more biomarkers associated with sensitivity to or toxicity of an anti-Trop-2 ADC;
c) selecting patients to be treated with an anti-Trop-2 ADC based on the presence of the one or more biomarkers; and
d) treating the selected patients with an anti-Trop-2 ADC.

4. The method of claim 3, further comprising:
e) selecting patients to be treated with a combination therapy, based on the presence of the one or more biomarkers; and
f) treating the patients with a combination of an anti-Trop-2 ADC and a DDR inhibitor.

5. The method of claim 3, wherein the an anti-Trop-2 ADC is administered to the patient as a neoadjuvant therapy, prior to administration of the at least one other anti-cancer therapy.

6. The method of claim 3, further comprising:
e) monitoring the patient for the presence of one or more biomarkers; and
f) determining the response of the cancer to the treatment.

7. The method of claim 6, further comprising monitoring for residual disease or relapse of the patient based on biomarker analysis.

8.-16. (canceled)

17. The method of claim 3, wherein the biomarker is a genetic marker in a DNA damage repair (DDR) gene or an apoptosis gene.

18.-19. (canceled)

20. The method of claim 3, wherein the biomarkers comprise or consist of AEN, MSH2, MYBBP1A, SART1, SIRT1, USP28, CDKN1A, ABL1, TP53, BAG6, BRCA1, BRCA2, BRSK2, CHEK2, ERN1, FHIT, HIPK2, HRAS, LGALS12, MSH6, ZNF385B and ZNF622.

21. The method of claim 3, wherein the biomarkers comprise or consist of BRCA1, BRCA2, CHEK2, MSH2, MSH6, TP53, CDKN1A, BAG6, BRSK2, ERN1, FHIT, HIPK2, LGALS12, ZNF622, AEN, SART1 and USP28.

22. The method of claim 3, wherein the biomarkers comprise or consist of POLR2K, DDB2, GADD45B, WEE1, TGFB1, NDRG1 and PPP1R15A.

23. The method of claim 3, wherein the biomarkers comprise or consist of GADD45B, TGFB1, NRG1, WEE1 and PPP1R15A.

24. The method of claim 3, wherein the biomarkers comprise or consist of BRCA1, BRCA2, CHEK2, MSH2, MSH6, TP53, CDKN1A, BAG6, BRSK2, ERN1, FHIT, HIPK2, LGALS12, ZNF622, AEN, SART1, USP28, GADD45B, TGFB1, NRG1, WEE1 and PPP1R15A.

25. (canceled)

26. The method of claim 3, wherein the biomarkers comprise or consist of BRCA1, BRCA2, PTEN, ERCC1 and ATM.

27. (canceled)

28. The method of claim 3, wherein the biomarkers are a plurality of single nucleotide polymorphisms that result in a substitution comprising or consisting of E155K in ABL1, G706S in ABL1, V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, G12V in HRAS, A278V in LGALS12, N127S in MSH2, S625F in MSH6, H680Y in MYBBP1A, R373Q in SART1, E113Q in SIRT1, *394S in TP53, R282G in TP53, T377P in in TP53, E271K in TP53, Y220C in TP53, E180* in TP53, I987L in USP28, R370Q in ZNF385B and A437E in ZNF622.

29. The method of claim 3, wherein the biomarkers are a plurality of single nucleotide polymorphisms that result in a substitution comprising or consisting of V172L in AEN, R279Q in BAG6, P1020Q in BRCA1, E255K in BRCA1, L2518V in BRCA2, T656A in BRSK2, M1V in CDKN1A, A377D in CHECK2, G771S in ERN1, R46S in FHIT, E457Q in HIPK2, N127S in MSH2, S625F in MSH6, R373Q in SART1, *394S in TP53, R282G in TP53, T377P in in TP53, E271K in TP53, Y220C in TP53, E180* in TP53, and I987L in USP28.

30. (canceled)

31. The method of claim 3, wherein the biomarkers are a plurality of frameshift mutations comprising or consisting of K1110fs in BAG6, R32fs in CDKN1A, DC33fs in CDKN1A, and EG60fs in CDKN1A.

32. (canceled)

33. The method of claim 3, wherein the biomarkers are a plurality of increases or decreases in gene expression in the cancer compared to corresponding normal tissue comprising or consisting of POLR2K, DDB2, GADD45B, WEEP TGFB1, NDRG1, and PPP1R15A.

34.-35. (canceled)

36. The method of claim 3, wherein the topoisomerase I inhibitor is SN-38 or DxD.

37. The method of claim 3, wherein the anti-Trop-2 ADC is selected from the group consisting of sacituzumab govitecan and DS-1062.

38.-52. (canceled)

53. The method of claim 3, wherein the cancer is urothelial cancer.

54. The method of claim 3, wherein the cancer is metastatic urothelial cancer.

55. The method of claim 3, wherein the cancer is treatment resistant urothelial cancer.

56. The method of claim 3, wherein the cancer is resistant to treatment with platinum-based and/or checkpoint inhibitor (CPI) (e.g., anti-PD1 antibody or anti-PD-L1 antibody) based therapy.

57.-90. (canceled)


SEQUENCE LISTING (6 sequences)

SEQ ID NO: 1 - protein, 11 aa, Artificial Sequence
  Description of Artificial Sequence Synthetic peptide
KASQDVSIAVA

SEQ ID NO: 2 - protein, 7 aa, Artificial Sequence
  Description of Artificial Sequence Synthetic peptide
SASYRYT

SEQ ID NO: 3 - protein, 9 aa, Artificial Sequence
  Description of Artificial Sequence Synthetic peptide
QQHYITPLT

SEQ ID NO: 4 - protein, 5 aa, Artificial Sequence
  Description of Artificial Sequence Synthetic peptide
NYGMN

SEQ ID NO: 5 - protein, 17 aa, Artificial Sequence
  Description of Artificial Sequence Synthetic peptide
WINTYTGEPTYTDDFKG

SEQ ID NO: 6 - protein, 12 aa, Artificial Sequence
  Description of Artificial Sequence Synthetic peptide
GGFGSSYWYFDV